(19) United States
(12) Patent Application Publication
(10) Pub. No.: US 2025/0188162 A1
(43) Pub. Date: Jun. 12, 2025

(54) INHIBITOR OF INTERLEUKIN-1 RECEPTOR TYPE 1 FOR USE IN THE TREATMENT OF CANCER

(71) Applicant: Buzzard Pharmaceuticals AB, Stockholm (SE)

(72) Inventor: Georgios Theodoros STATHOPOULOS, Munich (DE)

(21) Appl. No.: 18/845,676

(22) PCT Filed: Mar. 10, 2022
(86) PCT No.: PCT/EP2022/056269

§ 371 Date: Sep. 10, 2024

(51) Int. Cl. C07K 16/24; A61K 38/20; A61P 35/00

(52) U.S. Cl. CPC C07K 16/245; A61K 38/2006; A61P 35/00

(57) ABSTRACT

The invention provides an inhibitor for use in treating KRAS-mutant cancers, uses, and methods for treating KRAS-mutant cancers, methods for selecting subjects predicted to respond therapeutically to a treatment comprising the inhibitor. The invention also provides kits.

The invention provides an inhibitor for use in treating KRAS-mutant cancers, uses, and methods for treating KRAS-mutant cancers, and methods for selecting subjects predicted to respond therapeutically to a treatment comprising the inhibitor. The invention also provides kits.

Cancer is the leading cause of death worldwide, and its treatment and outcomes have been dramatically revolutionised by targeted therapies. As the most frequently mutated oncogene, Kirsten rat sarcoma viral oncogene homologue (KRAS) has attracted substantial attention.

Numerous clinical studies have shown that targeted therapies significantly extend progression-free survival and are less toxic than standard chemotherapy. For example, targeted therapies in patients with epidermal growth factor receptor (EGFR)-sensitive mutations or anaplastic lymphoma kinase (ALK) gene fusions have markedly enhanced survival time. Unfortunately, despite 40 years of proprietary drug efforts, there are still no effective strategies targeting KRAS mutations, except for sotorasib, which has just been approved to target the mutated KRAS (KRAS G12C). Due to the intrinsic characteristics of KRAS proteins, KRAS has been deemed a challenging therapeutic target, and even “undruggable. Therefore, many efforts have focused on indirectly targeting KRAS, by targeting its downstream signalling effectors, using epigenetic approaches such as telomerase inhibitors, and RNA interference. However, most of these strategies have failed due to a lack of activity or selectivity. In addition, patients with KRAS mutations usually have a poor response to current standard therapy. There is thus an urgent and unmet need to target KRAS-driven cancer.

Against this background, the inventor has surprisingly found that KRAS-mutant cancers activate NF-κB in tumor-associated myeloid cells in order to elicit the IL-1β they require for sustained growth. As shown in the accompanying Examples, IL-1β is elicited from macrophages through versican (VCAN).

In a first aspect, the invention provides an inhibitor of interleukin-1 receptor type 1 (IL-1R1) signalling for use in the treatment of cancer in a subject, wherein the cancer comprises a KRAS mutation and an elevated level and/or activity of veriscan (VCAN).

This aspect of the invention also includes use of an inhibitor of interleukin-1 receptor type 1 (IL-1R1) signalling in the manufacture of a medicament for treating a cancer in a subject, wherein the cancer comprises a KRAS mutation and an elevated level and/or activity of veriscan (VCAN).

This aspect of the invention also includes a method of treating a cancer in a subject, wherein the cancer comprises a KRAS mutation and an elevated level and/or activity of veriscan (VCAN), comprising administering an inhibitor of interleukin-1 receptor type 1 (IL-1R1) signalling to the subject.

Interleukin-1 receptor type 1 (IL-1R1), also termed IL-1RI, is a membrane-bound protein that regulates the inflammatory response through agonistic and antagonistic modulation of cytokine activity. Interleukin-1 alpha (IL-1α) and beta (IL-1β) are prototypic members of the IL-1 family of cytokines. Within the complex regulatory networks of IL-1 pathways, the cytokines IL-1α and IL-1β interact with the extracellular domain of IL-1R1, triggering the recruitment of an accessory receptor, the IL-1RAcP, resulting in a functional receptor complex that initiates IL-1R1 signaling cascades. Besides agonists IL-1α and IL-1β, IL-1R1 also binds an antagonist, IL-1Ra, which is not able to trigger IL-1R1 association with IL-1RAcP, thereby competitively blocking IL-1 signaling through IL-1R1 binding.

By “an inhibitor of interleukin-1 receptor type 1 (IL-1R1) signalling” we include the meaning of any molecule which can downregulate, antagonize, suppress, reduce, prevent, decrease, block, and/or reverse a biological activity and/or effect of IL-1R1. For the avoidance of doubt, we also include any molecule that prevents or decreases the expression of an IL-1R1 agonist (such as IL-1β) and/or increases the degradation of an IL-1R1 agonist (such as IL-1β) and/or increases the expression of an IL-1R1 antagonist (such as IL-1RA).

Methods for evaluating IL-1RI inhibition are known in the art. For example, it is possible to assay the activity of the IL-1 agonist cytokines (such as IL-1α and IL-1β). Several exemplary assays for IL-1 activity are described in Boraschi et al. (2006) and include T cell proliferation assays, IL-6 and IL-8 production assays, and inhibition of calcium influx. In one exemplary assay, the ability of an inhibitor may be evaluated for its ability to inhibit IL-1β stimulated release of IL-6 from human fibroblasts. Inhibition of IL-1β-stimulated cytokine release in MRC5 cells is correlated with the inhibitor's ability to inhibit IL-1 mediated activity in vivo. Details of the assay are described in Dinarello et al., Current Protocols in Immunology, Ch. 6.2.1-6.2.7, John Wiley and Sons Inc., 2000. Briefly, human MRC5 human fibroblasts (ATCC #CCL-171, Manassas VA, USA) are grown to confluency in multi-well plates. Cells are treated with titrated doses of inhibitor and controls. Cells are subsequently contacted with 100 μg/ml of IL-1β in the presence of the titrated inhibitor and/or controls. Negative control cells are not stimulated with IL-1β. The amounts of IL-6 released in each group of treated cells is measured using an IL-6 ELISA kit (e.g., BD Pharmingen, Franklin Lakes, NJ, USA). Controls that can be used include buffer alone, IL-1Ra, and antibodies to IL-1β. Efficacy of an inhibitor can also be evaluated in vivo. An exemplary assay is described in Economides et al., Nature Med., 9:47-52 (2003). Briefly, mice are injected intraperitoneally with titrated doses of the inhibitor and controls. Twenty-four hours after injection, mice are injected subcutaneously with recombinant human IL-1β at a dose of 1 μg/kg. Two hours after injection of the IL-1β (peak IL-6 response time), mice are sacrificed, and blood is collected and processed for serum.

IL-1 induces release of inflammatory mediators such as IL-6 from via IL-1R1. Serum IL-6 levels can be assayed by ELISA. Percent inhibition can be calculated based on the ratio of IL-6 detected in experimental animal serum to IL-6 detected in controls. Other exemplary assays for IL-1R1 activity in vivo are described in Boraschi et al. and include an anorexia, hypoglycaemia, and neutrophilia assay.

A further example of assaying IL-1R1 activity is described in Example 3 of WO 2012/016203 (incorporated by reference).

We include the meaning that signalling via IL-1R1 is reduced in the presence of the inhibitor, compared to the signaling via IL-1R1 in the absence of the inhibitor. Inhibition is not limited to complete inhibition or prevention of IL-1R1 signalling. In a given application, it may be that some low level of IL-1R1 signalling can be tolerated that will not have a detrimental effect on the outcome of the patient. In an embodiment, the inhibitor reduces IL-1R1 signalling by at least 10%, such as at least 20%, 30%, 40% or 50% compared to the signaling via IL-1R1 in the absence of the inhibitor. In an embodiment, the inhibitor reduces IL-1R1 signalling by at least 50%, such as at least 60%, 70%, 80% or 90%, such as by 95% compared to the signaling via IL-1R1 in the absence of the inhibitor.

The inhibitor may inhibit IL-1R1 signaling with an IC50 of less than 100, 50, 20, 10, or 5 nM.

There are two general mechanisms of inhibiting IL-1R1 signalling: binding to the IL-1 receptor (e.g. Isunakinra) or binding directly to an agonist IL-1 cytokine (e.g. rilonacept and canakinumab). By binding to the receptor, an inhibitor can prevent downstream signalling, for example, by reducing or preventing recruitment of IL-1RAcP. By binding directly to an agonist IL-1 cytokine, the inhibitor effectively sequesters IL-1α and/or IL-1β thus preventing them binding to IL-1R1.

In an embodiment, the inhibitor may be one that selectively inhibits IL-1R1 signalling. For example, the inhibitor may inhibit and/or decrease a biological activity of IL-1R1 to a greater extent than it inhibits a biological activity of an unrelated cell surface receptor. Preferably, the agent inhibits a biological activity of IL-1R1 at least 5, or at least 10, or at least 50 times more than it inhibits a biological activity of another unrelated cell surface receptor. More preferably, the agent inhibits a biological activity of IL-1R1 at least 100, or at least 1,000, or at least 10,000 times more than it inhibits a biological activity of another unrelated cell surface receptor.

By “biological activity of IL-1R1” we include the biological action of IL-1R1, and this refers to any function(s) exhibited or performed by a naturally occurring, and/or wild type form of IL-1R1 as measured or observed in vivo (i.e. in the natural physiological environment of the protein) or in vitro (i.e. under laboratory conditions). Such actives include the induction of inflammatory mediators such as IL-6. Assays for measuring the induction of IL-6 are described herein. The biological activity of IL-1R1 can also be tested using an NF-κB reporter gene assay as described herein.

In an embodiment, the inhibitor is one that binds to IL-1R1 in order to inhibit the biological activity of IL-1R1. In an embodiment, the inhibitor is one that selectively binds to IL-1R1.

By an inhibitor that “selectively binds” to IL-1R1, we include the meaning that the inhibitor binds to IL-1R1 with a greater affinity than to an unrelated cell surface receptor. In an embodiment, the inhibitor binds to IL-1R1 with at least 5, or at least 10 or at least 50 times greater affinity than to the unrelated cell surface receptor. In an embodiment, the agent binds to IL-1R1 with at least 100, or at least 1,000, or at least 10,000 times greater affinity than to an unrelated cell surface receptor. Binding to IL-1R1 may be determined by methods well known in the art, including ELISA and surface plasma resonance (SPR) (such as those described in Example 4 of WO 2012/016203 (incorporated by reference).

It will be appreciated that inhibition of IL-1R1 signalling which follows binding of the inhibitor to IL-1R1 may be termed “direct inhibition”. For example, the inhibitor may occupy a binding pocket of IL-1R1, thus not allowing an agonist cytokine, like IL-1α and IL-1β, the opportunity to bind. Such binding inhibition can be determined using assays and methods well known in the art, for example using BIAcore chips with immobilised IL-1R1 and incubating with soluble IL-1β with and without the inhibitor to be tested. Such binding inhibition can also be determined using flow cytometry or an ELISA.

ELISA assays typically involves the use of enzymes giving a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

ELISA methods are well known in the art, for example see The ELISA Guidebook (Methods in Molecular Biology), 2000, Crowther, Humana Press, ISBN-13:978-0896037281 (the disclosures of which are incorporated by reference).

In an embodiment, the inhibitor does not bind to IL-1R1 in order to inhibit IL-1R1 signalling. It will be appreciated that this may be termed “indirect inhibition”. For example, the inhibitor may bind to agonist cytokines IL-1β and/or IL-1α thus disrupting the ability of IL-1β and/or IL-1α to bind to the cognate receptor IL-1RI, and so IL-1R1 signaling is inhibited. Alternatively, the molecule may be able to sequester naturally occurring IL-1α and/or IL-1β so that IL-1α and/or IL-1β are unable to bind to IL-1R1. Alternatively, the inhibitor may be able to prevent the recruitment of the accessory receptor IL-1RAcP to IL01R1 leading to no signaling.

Preferably, the inhibitor is selected from the group comprising: a peptide, a polypeptide, a nucleic acid molecule (such as an aptamer), a small molecule, an antibody, a peptidomimetic, a natural product, a monobody, or a carbohydrate.

By a “nucleic acid” also termed “oligonucleotide”, “nucleic acid sequence,” “nucleic acid molecule,” and “polynucleotide” we include a DNA sequence or analog thereof, or an RNA sequence or analog thereof.

In an embodiment, the nucleic acid inhibitor may be an aptamer. Aptamers can be considered chemical antibodies having the properties of nucleotide-based therapies. Aptamers are small nucleic acid molecules that bind specifically to molecular targets such as proteins. Unlike nucleic acid therapeutics that act by hybridizing to another nucleic acid target, aptamers form three-dimensional shapes that allow for specific binding to enzymes, growth factors, receptors, viral proteins, and immunoglobulins. A nucleic acid aptamer generally includes a primary nucleotide sequence that allows the aptamer to form a secondary structure (e.g., by forming stem loop structures) that allows the aptamer to bind to its target. In the context of the present invention, aptamers can include DNA, RNA, nucleic acid analogues (e.g., peptide nucleic acids), locked nucleic acids, chemically modified nucleic acids, or combinations thereof. Aptamers can be designed for a given ligand by various procedures known in the art (see Buglak et al., Int J Mol Sci. 2020 Nov. 10; 21(22):8420)

In an embodiment, the inhibitor is a small molecule, including but not limited to small synthetic organic molecules which can inhibit IL-1R1 signalling. Their molecule weight usually is less than 800 Da and they possess properties, including good solubility, bioavailability, PK/PD, metabolism, etc.

By “peptide” we include short chains of amino acid monomers linked by peptide (amide) bonds. By “polypeptide” we include a long, continuous, and unbranched peptide chain.

In an embodiment, the inhibitor is an antibody. Antibodies are characterized in that they comprise immunoglobulin domains and as such, they are members of the immunoglobulin superfamily of proteins. By “antibody” we include the meaning of fragments thereof. Antibody fragments are portions of an intact full length antibody, such as an antigen binding fragments or variable region(s) of the intact antibody. Examples of antibody fragments include Fab, Fab′, F(ab′)2, and Fv fragments; diabodies; linear antibodies; single-chain antibody molecules (e.g., scFv); multispecific antibody fragments such as bispecific, trispecific, and multispecific antibodies (e.g., diabodies, triabodies, tetrabodies); minibodies; chelating recombinant antibodies; tribodies or bibodies; intrabodies; nanobodies; small modular immunopharmaceuticals (SMIP), binding-domain immunoglobulin fusion proteins; camelized antibodies; VHH containing antibodies; and any other polypeptides formed from antibody fragments.

By “antibody” and “antibodies” we include the meaning of polyclonal antibodies, monoclonal antibodies, humanized or chimeric antibodies, single chain Fv antibody fragments (scFv), single variable domains (VhH), Fab fragments, and F (ab) 2 fragments. Polyclonal antibodies are heterogeneous populations of antibody molecules that are specific for a particular antigen, which can be obtained from the sera of immunized animals. Polyclonal antibodies are produced using well-known methods. Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al. Nature. 1975, 256:495, the human B-cell hybridoma technique (Kosbor et al. Immunology Today, 1983, 4:72; Cole et al. Proc. Natl. Acad. Sci. USA. 1983, 80:2026), and the EBV-hybridoma technique (Cole et al. “Monoclonal Antibodies and Cancer Therapy”, Alan R. Liss, Inc., 1983, pp. 77-96). Such antibodies can be of any immunoglobulin class including IgG, IgM, IgE, IgA, IgD, and any subclass thereof. The hybridoma producing monoclonal antibodies can be cultivated in vitro or in vivo. A chimeric antibody is a molecule in which different portions are derived from different animal species, such as those having a variable region derived from a murine monoclonal antibody and a human immunoglobulin constant region. Chimeric antibodies can be produced through standard techniques.

By “antibody” we also include an antibody mimetic. By “antibody mimetic” we include organic compounds that are not structurally related to antibodies but are capable of binding to a target in a manner analogous to that of the antigen-antibody interaction. They are usually artificial peptides or proteins with a molar mass of about 3 to 20 kDa. Affibodies are a type of antibody mimetic, and their protein scaffold is derived from the B-domain of staphylococcal protein A.

By “carbohydrate” we include macromolecules that consist of carbon, hydrogen, and oxygen. They are organic compounds organized in the form of aldehydes or ketones with multiple hydroxyl groups coming off the carbon chain.

By “monobody” we include the meaning of synthetic binding proteins constructed using a fibronectin type III domain (FN3) as a molecular scaffold.

By “a subject” we include the meaning of a patient, or individual in need of treatment and/or prevention of a disease or condition as described herein. The subject may be a vertebrate, such as a vertebrate mammal. In an embodiment, the subject is selected from the group comprising: a primate (for example, a human; a monkey; an ape); a rodent (for example, a mouse, a rat, a hamster, a guinea pig, a gerbil, a rabbit); a canine (for example, a dog); a feline (for example, a cat); an equine (for example, a horse); a bovine (for example, a cow); and/or a porcine (for example, a pig). Preferably the subject is human.

By “cancer comprises a KRAS mutation” we include the meaning of a cancer having an increase in the number of copies of a KRAS gene (amplification), having a KRAS gene fusion, having a KRAS rearragnment, and/or a KRAS mutation, such as a missense gene mutation or nonsense gene mutation. It will be appreciated that the KRAS mutation is an oncogenic mutation, such as one which drives tumor initiation and maintenance. Non-limiting examples of such KRAS gene mutations include missense mutation or nonsense mutation at codon 5, codon 12, codon 13, codon 14, codon 18, codon 19, codon 23, codon 31, codon 38, codon 59, codon 61, codon 62, codon 97, codon 117, codon 118, codon 119, codon 121, codon 140, codon 143, codon 145, codon 146, codon 151, codon 153, codon 168, codon 171, codon 180, codon 185, codon 187, codon 188 of KRAS gene. More specific examples thereof include, but are not limited to, p.E3K, p.K5E, p.Y5N, p.G10dup, p.A11_G12dup, p.G12A, p.G12C, p.G12D, p.G12R, p.G12S, p.G12V, p.G13C, p.G13D, p.G13R, p.G13V, p.V14I, p.S17T, p.A18N, p.L19F, p.I21R, p.Q22K, p.L23R, p.I24N, p.Q31*, p.D33E, p.P34L, p.I36M, p.D38N, p.D38Y, p.T58K, p.A59G, p.A59E, p.A59T, p.Q61E, p.Q61R, p.Q61H, p.Q61P, p.Q61K, p.Q61L, p.E62Y, p.E62K, p.E63K, p.R68M, p.R68S, p.R97I, p.Y71C, p.K88*, p.D92Y, p.E98*, p.P110S, p.K117N, p.M118V, p.D119N, p.P121H, p.R123*, p.A130V, p.R135T, p.P140H, p.D143G, p.S145L, p.A146T, p.A146P, p.A146V, p.G151A, p.D153V, p.A155D, p.T158A, p.E168fs, p.R164Q, p.I171M, p.I171Nfs*14, p.K176Q, p.K180del, p.K185fs, p.I187V, p.M188V, p.X2_splice, p.KRAS-LMNTD1, p.KRAS-SLC2A14 and the like. More specific examples of the KRAS mutation at codon 12 include, but are not limited to, p.G12A, p.G12C, p.G12D, p.G12R, p.G12S, p.G12V and the like.

The determination of a mutation status including KRAS gene mutation status may be performed using methods well known in the art, for example, by an in vitro method in which a step of determining the gene mutation status in the patient comprises taking a sample from the patient and then determining the gene mutation status of the sample. The sample may comprise, for example, at least one of serum, whole fresh blood, peripheral blood mononuclear cells, frozen whole blood, fresh plasma, frozen plasma, urine, saliva, skin, hair follicle, bone marrow, tumor tissue, tumor biopsy, or archived paraffin-embedded tumor tissue.

The status of a KRAS mutation may be, for example, at the level of genomic DNA, protein and/or mRNA transcript of KRAS gene.

The determination of a KRAS mutation may be performed using a method selected from the group comprising: (a) PCR; (b) RT-PCR; (c) FISH; (d) IHC; (e) immunodetection methods; (f) Western Blot; (g) ELISA; (h) radioimmuno assays; (i) immunoprecipitation; (j) FACS (k) HPLC; (1) surface plasmon resonance; (m) optical spectroscopy; and (n) mass spectrometry. Presence of KRAS gene mutation(s) can be further detected by any sequencing method, including dideoxy sequencing, pyrosequencing, PYROMARK (registered trademark), KRAS assays, and allele-specific PCR assay. The determination of the KRAS gene may be detected using kits known in the art, for example, RASKET kit (Trade name, MEBGEN), therascreen KRAS RGQ PCR Kit (Qiagen).

Versican (VCAN) is an extracellular matrix proteoglycan. VCAN is encoded by a single gene and is located on chromosome 5q12-14 in the human genome. The human VCAN gene is divided into 15 exons over 90-100 kb. By “veriscan (VCAN)” we include the meaning of all splice variants of VCAN, including known splice variants V0, V1, V2, V3 and V4.

By “an elevated level and/or activity of veriscan (VCAN)” we include the meaning of a level and/or activity of VCAN that is increased in the cancer of the subject compared to a level and/or activity of VCAN in a reference subject(s). This elevated level and/or activity may be in the cancer cell itself but since VCAN is secreted from cancer cells, the elevated level and/or activity may be in the tumour microenvironment, such as the surrounding blood vessels, immune cells, fibroblasts, signaling molecules and/or the extracellular matrix. By “cancer cell” we include the meaning of a cell that has uncontrolled cell growth, such as a tumour cell.

In one embodiment, the “reference subject” is the same subject. Thus, the cancer may have an elevated level and/or activity of VCAN when the level and/or activity of VCAN in the cancer is increased relative to the level and/or activity of VCAN in a non-cancerous sample or tissue from the subject. Alternatively, the cancer may have an elevated level and/or activity of VCAN when the level and/or activity of VCAN in the cancer is increased relative to the level and/or activity of VCAN in the tissue comprising the cancer at an earlier time point (e.g. before the onset of malignant disease, before the onset of treatment, or during treatment).

In an alternative embodiment, the “reference subject(s)” are one or more healthy subjects, such as subjects that do not have cancer, or else do not have the same type of cancer (e.g. one affecting the same tissue). The healthy subjects may be in the same age group and, optionally, of the same gender, as the subject having cancer.

In certain embodiments, the level and/or activity of VCAN is deemed to be elevated when it is increased in the cancer of the subject by at least 5%, for example, at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 41%, 42%, 43%, 44%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, ±91%, 92%, 93%, 94%, 95%, 96%, 97%, 98%, 99%, 100%, 125%, ±150%, 175%, 200%, 225%, 250%, 275%, 300%, 350%, 400%, 500% or at least 1000% compared to a level and/or activity of VCAN in a reference subject(s).

In certain embodiments, the level and/or activity of VCAN is deemed to be elevated when it is increased in the cancer of the subject by at least 10%, at least 25%, at least 30%, at least 40%, at least 50%, at least 60%, at least 75%, at least 80%, at least 90%, at least 100%, at least 200%, at least 300%, or at least 500% compared to a level and/or activity of VCAN in a reference subject(s).

In certain embodiments, the level and/or activity of VCAN is deemed to be elevated when it is increased in the cancer of the subject by at least 25%, at least 50%, at least 75%, at least 100%, or at least 200% compared to a level and/or activity of VCAN in a reference subject(s).

In a particular embodiment, the level and/or activity of VCAN is deemed to be elevated when it is increased in the cancer of the subject by least 50% compared to a level and/or activity of VCAN in a reference subject(s). In a particular embodiment, the level and/or activity of VCAN is deemed to be elevated when it is increased in the cancer of the subject by at least 100% compared to a level and/or activity of VCAN in a reference subject(s). In a particular embodiment, the level and/or activity of VCAN is deemed to be elevated when it is increased in the cancer of the subject by at least 200% compared to a level and/or activity of VCAN in a reference subject(s). In one embodiment, the level and/or activity of VCAN is deemed to be elevated when it is increased in the cancer of the subject by at least 1.5 or 2 times compared to a level and/or activity of VCAN in a reference subject(s).

In some embodiments, the level and/or activity of VCAN in a subject is compared to the normal level and/or activity of VCAN. By “normal level and/or activity of VCAN we include the average level and/or activity of VCAN in a reference subject(s).

It will be appreciated that when assessing whether the level and/or activity of VCAN is elevated, it may be desirable to normalise the level and/or activity of the VCAN in the sample, and compare the normalised level and/or activity of the VCAN with a normalised level and/or activity of the VCAN in a sample from a healthy subject. For example, the level and/or activity can be normalised to control for differences in volume or content of samples (e.g. cell number). For instance, the level and/or activity of VCAN may be normalised to the level and/or activity of another product in a cell, such as beta-actin.

The level and/or activity of VCAN may be elevated due to an alteration to VCAN. By an “alteration” to VCAN we include mutations, copy number alterations, rearrangements and/or fusions of the gene encoding VCAN. Alterations, including missense mutations, include those recited in the cancer genome atlas (TCGA) pan-cancer dataset.

The level and/or activity of VCAN can be measured by any method known in the art or described herein. The level and/or activity of VCAN can be determined directly, i.e. by measuring the protein level of VCAN, or by measuring the mRNA of VCAN. In one embodiment, the level of VCAN is the protein level of VCAN. In one embodiment, the level of VCAN is mRNA level of VCAN.

For example, the level of VCAN, such as in a tissue sample, can be determined by assessing (e.g., quantifying) transcribed RNA of VCAN in the sample using, e.g., Northern blotting, PCR analysis, real time PCR analysis, or any other technique known in the art or described herein. In one embodiment, the level of VCAN, such as in a tissue sample, can be determined by assessing (e.g., quantifying) mRNA of VCAN in the sample. The level of VCAN, such as in a tissue sample, can also be determined by assessing (e.g., quantifying) the level of protein of VCAN in the sample using, e.g., immunohistochemical analysis, Western blotting, ELISA, immunoprecipitation, flow cytometry analysis, or any other technique known in the art or described herein.

In one embodiment, the level of VCAN, such as in a tissue sample, is determined by assessing (e.g., quantifying) protein expression of VCAN in the sample using immunohistochemistry. As shown in the accompanying Examples, VCAN protein expression was significantly increased in lung adenocarcinoma (LUAD) compared with adjacent lung tissues (FIG. 4N). Antibodies for use in assays that measure the levels of VCAN in a sample (e.g., in a tissue sample (e.g., a sample of lung, kidney, skin, thymus, breast, pancreatic, thyroid, bladder, liver, cervix, endometrium, colon, rectum and/or stomach) are known in the art or could be readily developed using approaches known to those of skill in the art. Examples of antibodies that can be used in assays that measure the levels of VCAN in a sample include rabbit anti-versican (E-AB-36300; Elabscience, Wuhan, China), and anti-VCAN (ab19345; Abcam, London, UK; RRID:AB_444865).

In one embodiment, the level of VCAN, such as in a tissue sample, is determined by assessing (e.g., quantifying) mRNA expression of VCAN in the sample using qPCR. As shown in the accompanying Examples, VCAN mRNA expression was significantly increased in human MPE compared with benign pleural effusions (BPE) (FIG. 4O). Primers that can be used to determine mRNA expression of VCAN are included in Table 4.

The level and/or activity of VCAN can be determined indirectly, i.e. by measuring the level and/or activity of a molecule (e.g. protein or mRNA) that is known to be effected by the level and/or activity of VCAN. For example, the activity of NF-κB can be measured in order to determine if a level and/or activity of VCAN is increased in the cancer.

The activity of VCAN can be measured by any assay known in the art including, without limitation, a reporter gene assay (e.g., containing VCAN-responsive reporter gene construct), measuring induction of IKKβ, or any other bioactivity assay. Exemplary assays that can be used to measure activity of VCAN are described herein.

In an embodiment, an NF-κB reporter gene assay can be used to assess VCAN activity. Specifically, the NF-κB reporter Raw 264.7 cell line (comprising NFκB.GFP. Luciferase (NGL)) is designed for monitoring nuclear factor Kappa B (NF-κB) signal transduction pathways. It contains a firefly luciferase gene driven by four copies of the NF-κB response element located upstream of the minimal TATA promoter. After activation by pro-inflammatory cytokines or stimulants of lymphokine receptors, endogenous NF-κB transcription factors bind to the DNA response elements, inducing transcription of the luciferase reporter gene. As shown in the accompanying Examples, VCAN potently activates macrophage NF-κB-driven transcription (FIGS. 4F, G). Moreover, shRNA-mediated Vcan silencing diminished their ability to trigger NF-κB activation in the reporter gene assay (FIGS. 4I-M).

In an embodiment, the activity of VCAN is determined my measuring the induction of IKKβ by immunoblotting and/or by microarray. As shown in the accompanying Examples, VCAN induced IKKβ in primary murine bone marrow-derived macrophages (BMDMs) (FIG. 4H).

The level and/or activity of VCAN can be assessed in any tissue sample obtained from a subject in accordance with the methods described herein. In certain embodiments, VCAN level and/or activity is assessed in a sample obtained from lung, kidney, skin, thymus, breast, pancreatic, thyroid, bladder, liver, cervix, endometrium, colon, rectum and/or stomach of a subject in accordance with the methods described herein.

By “treat”, “treatment” and “treating” we include the meaning of the reduction or amelioration of the progression, severity and/or duration of a disorder, e.g., cancer, or the amelioration of one or more symptoms, suitably of one or more discernible symptoms, of the disorder resulting from the administration of one or more therapies. In specific embodiments, the terms “treat”, “treatment” and “treating” refer to the amelioration of at least one measurable physical parameter of cancer such as growth of a tumor, not necessarily discernible by the patient. In other embodiments the terms “treat”, “treatment” and “treating” refer to the inhibition of the progression of cancer, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count. Taking lung cancer as an example, the term treatment may refer to at least one of the following: alleviating one or more symptoms of lung cancer, delaying progression of lung cancer, shrinking tumor size in lung cancer patient, inhibiting lung cancer tumor growth, prolonging overall survival, prolonging progression free survival, preventing or delaying lung cancer tumor metastasis, reducing (such as eradiating) pre-existing lung cancer tumor metastasis, reducing incidence or burden of pre-existing lung cancer tumor metastasis, or preventing recurrence of lung cancer.

In an embodiment, the cancer has been determined as being one comprising a KRAS mutation.

In an embodiment, the cancer comprises an elevated level of VCAN mRNA and/or VCAN protein.

In an embodiment, the elevated level and/or activity of VCAN is a level and/or activity of VCAN that is elevated in a test sample from the subject relative to the level and/or activity of VCAN in a reference sample.

In an embodiment, the reference sample is taken from a reference subject as described herein.

It will be appreciated by persons skilled in the art that, in addition to measuring the activity of VCAN in a cancer of the subject, the methods of the invention may also comprise measuring that same activity of VCAN in one or more reference samples.

It will be appreciated by persons skilled in the art that, in addition to measuring the level of VCAN in a cancer of the subject, the methods of the invention may also comprise measuring the level of VCAN in one or more reference samples from a corresponding tissue. For example, if VCAN levels are measured in a test sample comprising lung tissue, it is preferable that VCAN levels are measured in a reference sample comprising lung tissue.

By “sample to be tested”, “test sample” or “reference sample” we include a tissue or fluid sample taken or derived from an individual, wherein the sample comprises endogenous proteins and/or nucleic acid molecules and/or carbohydrate moieties. The sample may be a cellular sample, a tissue sample a blood sample, a serum sample, or a sample of pleural or peritoneal fluids.

Preferably, the test sample comprises cancerous cells. The sample may be a biopsy sample taken from a subject, for example, one that contains suspected or known cancer cells. The sample may be archived paraffin-embedded tumor tissue.

In an embodiment, the cancer comprises an elevated level and/or activity of IL-1β.

By “an elevated level and/or activity of IL-1β” we include the meaning of a level and/or activity of IL-1β that is increased in the cancer of the subject compared to a level and/or activity of IL-1β in a reference subject(s). This elevated level and/or activity may be in the cancer cell itself but since IL-1β is secreted from myeloid cells, the elevated level and/or activity may be in the tumour microenvironment, such as the surrounding blood vessels, immune cells (such as macrophages), fibroblasts, signaling molecules and/or the extracellular matrix.

In one embodiment, the “reference subject” is the same subject. Thus, the cancer may have an elevated level and/or activity of IL-1β when the level and/or activity of IL-1β in the cancer is increased relative to the level and/or activity of IL-1β in a non-cancerous sample or tissue from the subject. Alternatively, the cancer may have an elevated level and/or activity of IL-1β when the level and/or activity of IL-1β in the cancer is increased relative to the level and/or activity of IL-1β in the tissue comprising the cancer at an earlier time point (e.g. before the onset of malignant disease, before the onset of treatment, or during treatment).

In an alternative embodiment, the “reference subject(s)” are one or more healthy subjects, such as subjects that do not have cancer, or else do not have the same type of cancer (e.g. one affecting the same tissue). The healthy subjects may be in the same age group and, optionally, of the same gender, as the subject having cancer.

In certain embodiments, the level and/or activity of IL-1β is deemed to be elevated when it is increased in the cancer of the subject by at least 5%, for example, at least 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 41%, 42%, 43%, 44%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, ±91%, 92%, 93%, 94%, 95%, 96%, 97%, 98%, 99%, 100%, 125%, ±150%, 175%, 200%, 225%, 250%, 275%, 300%, 350%, 400%, 500% or at least 1000% compared to a level and/or activity of IL-1β in a reference subject(s).

In certain embodiments, the level and/or activity of IL-1β is deemed to be elevated when it is increased in the cancer of the subject by at least 10%, at least 25%, at least 30%, at least 40%, at least 50%, at least 60%, at least 75%, at least 80%, at least 90%, at least 100%, at least 200%, at least 300%, or at least 500% compared to a level and/or activity of IL-1β in a reference subject(s). In certain embodiments, the level and/or activity of IL-1β is deemed to be elevated when it is increased in the cancer of the subject by at least 25%, at least 50%, at least 75%, at least 100%, or at least 200% compared to a level and/or activity of IL-1β in a reference subject(s).

In a particular embodiment, the level and/or activity of IL-1β is deemed to be elevated when it is increased in the cancer of the subject by least 50% compared to a level and/or activity of IL-1β in a reference subject(s). In a particular embodiment, the level and/or activity of IL-1β is deemed to be elevated when it is increased in the cancer of the subject by at least 100% compared to a level and/or activity of IL-1β in a reference subject(s). In a particular embodiment, the level and/or activity of IL-1β is deemed to be elevated when it is increased in the cancer of the subject by at least 200% compared to a level and/or activity of IL-1β in a reference subject(s). In one embodiment, the level and/or activity of IL-1β is deemed to be elevated when it is increased in the cancer of the subject by at least 1.5 or 2 times compared to a level and/or activity of IL-1β in a reference subject(s).

In some embodiments, the level and/or activity of IL-1β in a subject is compared to the normal level and/or activity of IL-1β. By “normal level and/or activity of IL-1β we include the average level and/or activity of IL-1β in a reference subject(s).

It will be appreciated that when assessing whether the level and/or activity of IL-1β is elevated, it may be desirable to normalise the level and/or activity of the IL-1β in the sample, and compare the normalised level and/or activity of the IL-1β with a normalised level and/or activity of the IL-1β in a sample from a healthy subject. For example, the level and/or activity can be normalised to control for differences in volume or content of samples (e.g. cell number). For instance, the level and/or activity of IL-1β may be normalised to the level and/or activity of another product in a cell, such as beta-actin.

The level and/or activity of IL-1β may be elevated due to an alteration to IL-1β. By an “alteration” to IL-1β we include mutations, copy number alterations, rearrangements, and/or fusions of the gene encoding IL-1β. Alterations, including missense mutations, include those recited in the cancer genome atlas (TCGA) pan-cancer dataset.

The level and/or activity of IL-1β can be measured by any method known in the art or described herein. The level and/or activity of IL-1β can be determined directly, i.e. by measuring the protein level of IL-1β, or by measuring the mRNA of IL-1β. In one embodiment, the level of IL-1β is the protein level of IL-1β. In one embodiment, the level of IL-1β is mRNA level of IL-1β.

For example, the level of IL-1β, such as in a tissue sample, can be determined by assessing (e.g., quantifying) transcribed RNA of IL-1β in the sample using, e.g., Northern blotting, PCR analysis, real time PCR analysis, or any other technique known in the art or described herein. In one embodiment, the level of IL-1β, such as in a tissue sample, can be determined by assessing (e.g., quantifying) mRNA of IL-1β in the sample. The level of IL-1β, such as in a tissue sample, can also be determined by assessing (e.g., quantifying) the level of protein of IL-1β in the sample using, e.g., immunohistochemical analysis, Western blotting, ELISA, immunoprecipitation, flow cytometry analysis, or any other technique known in the art or described herein.

In one embodiment, the level of IL-1β, such as in a tissue sample, is determined by assessing (e.g., quantifying) protein expression of IL-1β in the sample using ELISA. As shown in the accompanying Examples, the inventor have shown that KRAS mutation status correlates with IL-1β expression and protein levels (see FIG. 3L). Antibodies and reagents for use in assays that measure the levels of IL-1β in a sample (e.g., in a tissue sample, e.g., a sample of lung, kidney, skin, thymus, breast cancer, pancreatic, thyroid, bladder, liver, cervix, endometrium, colon, rectum and/or stomach) are known in the art or could be readily developed using approaches known to those of skill in the art. Examples of reagents that can be used in assays that measure the levels of IL-1β in a sample include IL-1β ELISA (catalogue #900-K47) from Peprotech (London, UK), and the R&D Systems high sensitivity IL-Ib ELISA kit.

In one embodiment, the level of IL-1β, such as in a tissue sample, is determined by assessing (e.g., quantifying) mRNA expression of IL-1β in the sample using qPCR. As shown in the accompanying Examples, IL-1β mRNA expression was increased in KRAS-mutant cancers (FIG. 3K) Primers that can be used to determine mRNA expression of VCAN are included in Table 4.

The level and/or activity of IL-1β can be determined indirectly, i.e. by measuring the level and/or activity of a molecule (e.g. protein or mRNA) that is known to be effected by the level and/or activity of IL-1β. For example, the IL-1β stimulated release of IL-6 from human fibroblasts can be measured, as described herein, in order to determine if a level and/or activity of IL-1β is increased in the cancer.

The activity of IL-1β can be measured by any assay known in the art including, without limitation, IL-1β stimulated release of IL-6 from human fibroblasts or any other bioactivity assay. Exemplary assays that can be used to measure activity of IL-1β are described herein.

The level and/or activity of IL-1β can be assessed in any tissue sample obtained from a subject in accordance with the methods described herein. In certain embodiments, IL-1β level and/or activity is assessed in a sample obtained from lung, kidney, skin, thymus, breast cancer, pancreatic, thyroid, bladder, liver, cervix, endometrium, colon, rectum and/or stomach of a subject in accordance with the methods described herein.

In an embodiment, the elevated level and/or activity of IL-1β is a level and/or activity of IL-1β that is elevated relative to the level and/or activity of IL-1β level in a reference sample.

In an embodiment, the cancer comprises an elevated level and/or activity of inhibitor of NF-κB kinase (IKK)β. As shown in the accompanying Examples, KRAS-mutant tumors trigger IKKβ activation and IL-1β release via secretory versican (see FIG. 4H), and also that the VCAN-IKKβ axis is required for sustained growth of KRAS-mutant tumors. Methods for determining whether a cancer comprises an elevated level and/or activity of IKKβ are described herein and include the same methods described in relation to VCAN and IL-1β. By “an elevated level and/or activity of IKKβ” we include the meaning of a level and/or activity of IKKβ that is increased in the cancer of the subject compared to a level and/or activity of IKKβ in a reference subject(s). The “elevated” level and/or activity is as defined in relation to VCAN and/or IL-1β. This elevated level and/or activity may be in the cancer cell itself but since IKKβ is expressed in myeloid cells, the elevated level and/or activity may be in the tumour microenvironment, such as the surrounding blood vessels, immune cells (such as macrophages), fibroblasts, signaling molecules and/or the extracellular matrix. As shown in the accompanying Examples, IKKβ signaling in primary macrophages is required for their differentiation and expression of critical proinflammatory genes including Il1b.

The level and/or activity of IKKβ may be elevated due to an alteration to IKKβ. By an “alteration” to IKKβ we include mutations, copy number alterations, rearrangements and/or fusions of the gene encoding IKKβ. Alterations, including missense mutations, include those recited in the cancer genome atlas (TCGA) pan-cancer dataset.

In an embodiment, the reference sample is a non-cancerous sample.

In an embodiment, the non-cancerous sample is a sample comprising non-cancerous cells or tissue from a subject.

In an embodiment, the non-cancerous sample is from the same subject or a different subject.

In an embodiment, determining whether the cancer comprises an elevated level and/or activity of veriscan (VCAN), an elevated level and/or activity of IL-1-1β, and/or an elevated level and/or activity of IKKβ is carried out in vitro. In other words, determining whether the cancer comprises an elevated level and/or activity of veriscan (VCAN), an elevated level and/or activity of IL-1-1β, and/or an elevated level and/or activity of IKKβ is carried out on a sample that has already been obtained from the subject.

In an embodiment, the cancer is selected from the group comprising lung cancer (such as non-small cell lung cancer (NSCLC)), kidney cancer, skin cancer, thymus cancer, breast cancer, pancreatic cancer, thyroid cancer, bladder cancer, liver cancer, cervical cancer, endometrial cancer, colorectal cancer, and stomach cancer.

In an embodiment, the cancer is one that causes malignant pleural effusion (MPE). By “malignant pleural effusion (MPE)” we include the meaning of a build-up of fluid and cancer cells that collects between the chest wall and the lung. Some types of cancer are more likely to cause a pleural effusion. For example, around 40% of people with lung cancer develop a pleural effusion at some point during the course of their cancer. Cancers that cause MPE include breast cancer, lung cancer, lymphoma, mesothelioma, and ovarian cancer.

In an embodiment, the cancer is selected from the group comprising: lung adenocarcinoma (LUAD), colon adenocarcinoma (COAD), rectal adenocarcinoma (READ), and uterine corpus endometrial carcinoma (UCEC).

In an embodiment, the inhibitor is selected from the group comprising: a peptide IL-1 receptor antagonist, a nucleotide IL-1 receptor antagonist, peptide fragments of IL-1R1, an anti-IL-1 antibody, an anti-IL-1R1 antibody, a decoy IL-1 receptor (optionally a soluble IL-1 receptor, or an IL-1 TRAP).

In an embodiment the inhibitor is a peptide that acts as an IL-1 receptor antagonist, such as an antagonistic cytokine. Antagonistic cytokines bind to IL-1R1 yet do not allow the accessory receptor to form the necessary trimeric complex, thus prohibiting IL-1 signaling. Naturally occurring and modified forms of IL-1RA can be useful for inhibiting IL-1R1 signalling.

The IL-1 receptor antagonist (IL-1RA; also called IRAP, for IL-1 receptor antagonist protein) acts as a natural antagonist of IL-1α and IL-1β by binding to the IL-1 receptor but not transducing an intracellular signal or a biological response. IL-1RA is an antagonistic ligand because it does not interact with the accessory receptor, IL-1RAcP, and hence cannot signal. The gene encoding this antagonist of IL-1 has been described (see Hannum et al. (1990) Nature 343:336-340; Eisenberg et al. (1990) Nature 343:341-346; and Carter et al. (1990) Nature 344:633-638). The peptide IL-1RA inhibits the biological activities of IL-1 both in vitro and in vivo, and has been shown to be effective in animal models of septic shock, rheumatoid arthritis, graft versus host disease, stroke, and cardiac ischemia. Normal animals, including humans, can be infused intravenously with high doses of this protein without any change in physiological or metabolic parameters. For example, human volunteers infused with 133 mg/h IL-1RA for 72 hours exhibited no change in clinical or laboratory values. See Dinarello et al. (1993) J. Amer. Med. Assoc. 269:1829-1835. Thus, in an embodiment the inhibitor is naturally occurring IL-1RA, such as human IL-1RA.

In an embodiment the inhibitor is a modified form of IL-1RA. In an embodiment, the inhibitor is r-metHuIL-1ra (also known as Anakinra, Kineret®), a recombinant version of the naturally occurring IL-1 receptor antagonist. Anakinra is a non-glycosylated form of human IL-1RA that competitively inhibits IL-1α and IL-1β from binding to IL-1 receptor type 1. This polypeptide is 153 amino acids in length, has a molecular weight of 17.3 kDa, and except for the addition of an N-terminal methionine, is identical to the naturally occurring, non-glycosylated form of human IL-1RA.

In an embodiment, the disclosure provides a peptide inhibitor that includes an amino acid sequence at least 80, 82, 85, 87, 88, 89, 90, 91, 92, 93, 94, 95, 96, 97, 98, 99% or 100% identical to a sequence disclosed herein, e.g., a sequence listed in Table 6 or in the Examples, and/or in WO 2012/016203, which is incorporated by reference in its entirety.

TABLE 6

| | | SEQ |
|---|---|---|
| | Exemplary chimeric proteins | ID NO: |
| P01 | APVRSLAFRIWDVNQKTFYLRNNQLVAGYLQGPNVNLEEKIDVS | 1 |
| | FVQGEESNDKIPVALGIHGGKMCLSCVKSGDETRLQLEAVDPK | |
| | NYPKKKMDKRFAFIRSDSGPTTSFESAACPGWFLCTAMEADQP | |
| | VSLTNMPDEGVMVTKFYMQFVSS | |
| P02 | APVRSLAFRIWDVNQKTFYLRNNQLVAGYLQGPNVNLEEKIDVS | 2 |
| | FVQGEESNDKIPVALGIHGGKMCLSCVKSGDETRLQLEAVDPK | |
| | NYPKKKMEKRFVFNKIEINNKLSFESAACPGWFLCTAMEADQPV | |
| | SLTNMPDEGVMVTKFYMQFVSS | |
| P03 | APVRSLAFRIWDVNQKTFYLRNNQLVAGYLQGPNVNLEEKFSM | 3 |
| | SFVQGEESNDKIPVALGLKEKNLYLSCVLKDDKPTLQLESVDPK | |
| | NYPKKKMEKRFVFIRSDSGPTTSFESAACPGWFLCTAMEADQP | |
| | VSLTNMPDEGVMVTKFTMQFVSS | |
| P04 | APVRSLAFRIWDVNQKTFYLRNNQLVAGYLQGPNVNLEEKFSM | 4 |
| | SFVQGEESNDKIPVALGLKEKNLYLSCVLKDDKPTLQLESVDPK | |
| | NYPKKKMEKRFVFNKIEINNKLEFESAACPGWFLCTAMEADQPV | |
| | SLTNMPDEGVMVTKFTMQFVSS | |
| P05 | APVRSLNCRIWDVNQKTFYLRNNQLVAGYLQGPNVNLEEKFSM | 5 |
| | SFVQGEESNDKIPVALGLKEKNLYLSCVLKDDKPTLQLESVDPK | |
| | NYPKKKMEKRFVFNKIEINNKLEFESAQFPNWFLCTAMEADQPV | |
| | SLTNMPDEGVMVTKFYMQFVSS | |

P01. P01 comprises three segments from IL-1Ra (SEQ ID NO: 6) corresponding to amino acids Ala12-Val48, Ile60-Val83, and Asp95-Tyr147 of SEQ ID NO:6, and the remaining four segments from IL-1β (SEQ ID NO: 7). Overall, P01 has 74 of 153 amino acids from IL-1β (about 48% identity) and 119 amino acids from IL-1Ra (about 77% identity). These percentages add up to greater than 100% because a number of amino acids in P01 and other exemplary proteins disclosed herein are amino acids that are conserved between IL-1β and IL-1Ra and accordingly contribute to the percentage identity for both IL-1β and IL-1Ra.

The amino acid sequence of IL-1Ra (human) as referenced herein is:

(SEQ ID NO: 6)
RPSGRKSSKMQAFRIWDVNQKTFYLRNNQLVAGYLQGPNVNLEEKIDVV
PIEPHALFLGIHGGKMCLSCVKSGDETRLQLEAVNITDLSENRKQDKRF
AFIRSDSGPTTSFESAACPGWFLCTAMEADQPVSLTNMPDEGVMVTKFY
FQED.

The amino acid sequence of IL-1β (human) as referenced herein is:

(SEQ ID NO: 7)
APVRSLNCTLRDSQQKSLVMSGPYELKALHLQGQDMEQQVVFSMSFVQG
EESNDKIPVALGLKEKNLYLSCVLKDDKPTLQLESVDPKNYPKKKMEKR
FVFNKIEINNKLEFESAQFPNWYISTSQAENMPVFLGGTKGGQDITDFT
MQFVSS.

P02. P02 comprises three segments from IL-1Ra corresponding to amino acids Ala12-Val48, Ile60-Val83, and Ser110-Tyr147 of SEQ ID NO:6, and the remaining four segments from IL-1β. Overall, P02 has 85 of 153 amino acids from IL-1β (about 55% identity) and 108 amino acids from IL-1Ra (about 70% identity).

P03. P03 comprises two segments from IL-1Ra corresponding to amino acids Ala12-Lys45 and Phe100-Lys145 of SEQ ID NO:6, and the remaining three segments from IL-1β. Overall, P03 has 94 of 153 amino acids from IL-1β (about 61% identity) and 91 amino acids from IL-1Ra (about 64% identity).

P04. P04 comprises two segments from IL-1Ra corresponding to amino acids Ala12-Lys45 and Ala114-Lys145 of SEQ ID NO:6, and the remaining three segments from IL-1β. Overall, P04 has 104 of 153 amino acids from IL-1β (about 68% identity) and 89 amino acids from IL-1Ra (about 58% identity).

P05. P05 comprises two segments from IL-1Ra corresponding to amino acids Arg14-Lys45 and Phe120-Tyr147 of SEQ ID NO:6, and the remaining three segments from IL-1β. Overall, P05 has 108 of 153 amino acids from IL-1β (about 70% identity) and 85 amino acids from IL-1Ra (about 55% identity).

The peptide inhibitor can include a range of different residues from IL-1β and IL-1Ra as illustrated below in Table 7. In an embodiment, the peptide inhibitor can have 48-70% residues from IL-1β and 55-78% residues from IL-1Ra. In an embodiment, the peptide inhibitor can have 48-72% residues from IL-1β and 55-78% residues from IL-1Ra. (Because a number of amino acid residues are conserved between the two proteins, the sum of the percentage identity to IL-1β and to IL-1Ra can be greater than 100%.)

TABLE 7

| | IL-1β | IL-1RA | Total | | |
|---|---|---|---|---|---|
| | residues | residues | residues | % IL-1β | % IL-1RA |
| P05 | 108 | 85 | 153 | 70.6 | 55.6 |
| P04 | 104 | 89 | 153 | 68.0 | 58.2 |
| P03 | 94 | 99 | 153 | 61.4 | 64.7 |
| P02 | 85 | 108 | 153 | 55.6 | 70.6 |
| P01 | 74 | 119 | 153 | 48.4 | 77.8 |

In an embodiment, the inhibitor is between 45-72% identical to IL-1β and 53-80% identical to IL-1Ra; between 50-72% identical to IL-1β and 53-71% identical to IL-1Ra; between 60-72% identical to IL-1β and 53-68% identical to IL-1Ra; between 65-72% identical to IL-1β and 54-60% identical to IL-1Ra; or between 68-72% identical to IL-1β and 54-57% identical to IL-1Ra.

The peptide inhibitor may include a methionine N-terminal to the amino acid sequence of P01, P02, P03, P04, or P05, and/or the peptide inhibitors may include the amino acid sequence of P01, P02, P03, P04, or P05 in which the alanine at N-terminus is absent.

The peptide inhibitor may include a tag, such as a hexa-histidine sequence, such as GSHHHHHH. The tag can be N- or C-terminal relative to the inhibitor sequence. In an embodiment, the hexa-histidine tag is C-terminal to the inhibitor sequence.

In an embodiment, the inhibitor is at least 80% identical to a sequence selected from any one of SEQ ID NO: 1 (P01), SEQ ID NO: 2 (P02), SEQ ID NO: 3 (P03), SEQ ID NO: 4 (P04) and SEQ ID NO: 5 (P05).

Calculations of “homology” or “sequence identity” between two sequences (the terms are used interchangeably herein) can be performed as follows. The sequences are aligned according to the alignments provided herein, or, in the absence of an appropriate alignment, the optimal alignment determined as the best score using, for example, Needleman and Wunsch algorithm as implemented in the Needle algorithm of the EMBOSS package using a Blosum 62 scoring matrix with a gap penalty of 10, and a gap extend penalty of 1. See Needleman, S. B. and Wunsch, C. D. (1970) J. Mol. Biol. 48, 443-453; Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley, and tools available from the European Bioinformatics Institute (Cambridge UK) EMBOSS: The European Molecular Biology Open Software Suite (2000), Rice, P. et al., A., Trends in Genetics 16, (6) pp. 276-277 and available online at https://www.ebi.ac.uk/Tools/psa/. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences. To determine collective identity of one sequence of interest to a group of reference sequences, a position is considered to be identical if it is identical to at least one amino acid at a corresponding position in any one or more of the group of reference sequences. With respect to lists of segments, features, or regions, identity can be calculated collectively for all members of such list to arrive an overall percentage identity.

As used herein, the term “corresponding to” is used to designate the position of an amino acid residue in a polypeptide of interest with respect to a reference polypeptide. In general the position is the one indicated by an alignment such as in FIG. 4 of WO 2012/016203, which is incorporated by reference in its entirety.

In an embodiment, the inhibitor is at least 90% identical to a sequence selected from any one of SEQ ID NO: 1 (P01), SEQ ID NO: 2 (P02), SEQ ID NO: 3 (P03), SEQ ID NO: 4 (P04) and SEQ ID NO: 5 (P05).

In an embodiment, the inhibitor is at least 95% identical to P01, P02, P03, P04, or P05. In an embodiment, the inhibitor is identical to P01. In an embodiment, the inhibitor is identical to P02. In an embodiment, the inhibitor is identical to P03. In an embodiment, the inhibitor is identical to P04. In an embodiment, the inhibitor is identical to P05.

In an embodiment, the inhibitor is Isunakinra.

By “Isunakinra” we include the meaning of a protein chimera of IL-1β and IL-1 receptor antagonists comprising P05 (SEQ ID NO: 5), and as described in WO 2012/016203, incorporated by reference.

Preferred formulations of Isunakinra are described in WO 2014/160371, which is incorporated by reference in its entirety.

In addition to antagonist peptide cytokines (such as Isunakinra and Anakinra), it is possible to use low molecular weight peptides that bind IL-1R1 and act as antagonists. One such peptide, AF10847, was crystalized with IL-1RI to determine its mechanism of antagonism, showing that this peptide bound site A of IL-1RI and induced a conformational change in the receptor that renders it incapable of cytokine binding (see Vigers G P, Dripps D J, Edwards CK, 3rd, Brandhuber B J. X-ray crystal structure of a small antagonist peptide bound to interleukin-1 receptor type 1. J Biol Chem. (2000) 275:36927-33).

In an embodiment the inhibitor is a nucleotide that acts as an IL-1 receptor antagonist, such as an RNA or DNA aptamer. Aptamers are oligonucleotide fragments that can bind protein targets. The DNA aptamer SL1067 binds IL-1a and disrupts its ability to bind to its cognate receptor IL-1RI (see Ren X, Gelinas A D, Von Carlowitz I, Janjic N, Pyle A M. Structural basis for IL-1alpha recognition by a modified DNA aptamer that specifically inhibits IL-1alpha signaling. Nat Commun. (2017) 8:810).

In an embodiment the inhibitor is a decoy IL-1 receptor. By “decoy IL-1 receptor” we include the meaning of a synthetic or naturally occurring IL-1 receptor which can bind the IL-1 cytokines but lacks the cytoplasmic domain necessary for signaling. The active receptor complex consists of the type I receptor (IL-1RI) and IL1RAcP (for IL-1 receptor accessory protein). IL-1RI is responsible for binding of the three naturally occurring ligands (IL-1α, IL-1β and IL-1RA) and is able to do so in the absence of the IL1RAcP. However, signal transduction requires interaction of IL-1α or IL-1β with the IL1RAcP, therefore upon binding of IL-1β or IL-1α to IL-1RI, IL-1RAcP is recruited to initiate signaling. Such decoy receptors may therefore act by sequestering IL-1α or IL-1β from active IL-1R1. Such decoy receptors may not be attached to the plasma membrane and therefore be soluble, this can allow the decoy receptors to sequester free cytokines thus preventing binding to cell-surface expressed IL-1R1.

Such decoy receptors may sequester the accessory protein (IL-1RAcP) thus preventing it participating in IL-1 signaling. The IL-1 type II receptor (IL-1RII) can bind IL-1 agonists (IL-1α and IL-1β), it subsequently may recruit the IL-1RAcP for the creation of the IL-1 ternary complex but due to its lack of an intracellular TIR domain no signaling can occur. IL-1RII is a decoy receptor, either in its membrane form or as an antagonist in a cleaved secreted form, which can inhibit IL-1 activity. For a review, see Dinarello (1996) Blood 87:2095-2147.

By “IL-1 TRAP” we include the meaning of a recombinant nucleic acid molecule encoding a fusion polypeptide which forms a multimer capable of binding interleukin-1 (IL-1) to form a non-functional complex. An IL-1 TRAP is as essentially described in WO2004039951A2. IL-1 TRAP is a decoy receptor that can bind to circulating IL-1α and IL-1β molecules, effectively blocking the engagement of IL-1β to IL-1R1, inhibiting the downstream activation of the downstream signalling pathway. The IL-1 TRAP incorporates into a single molecule the extracellular domains of both receptor components required for IL-1 signaling; the IL-1 Type I receptor (IL-1RI) and the IL-1 receptor accessory protein (AcP). Since it contains both receptor components, the IL-1 TRAP binds IL-1β and IL-1α with picomolar affinity, while the IL-1RI alone in the absence of AcP binds with an affinity constant of about 1 nanomolar. The IL-1 TRAP was created by fusing the sequences encoding the extracellular domains of the AcP, IL-1RI, and Fc inline without any intervening linker sequences. An expression construct encoding the fusion protein is transfected into Chinese hamster ovary (CHO) cells, and high producing lines are isolated that secrete the IL-1 TRAP into the medium. The IL-1 TRAP is a dimeric glycoprotein with a protein molecular weight of 201 kDa and including glycosylation has a total molecular weight of ˜252 kDa. The dimer is covalently linked by disulfide bonds in the Fc region. Such IL-1 TRAPS are able to neutralize IL-1 receptor signaling by acting as a decoy receptor. An example is Rilonacept (trade name Arcalyst, Regeneron Pharmaceuticals).

In an embodiment, the inhibitor is an IL-1 antibody such as an anti-IL-1β antibody and/or an anti-IL-1α antibody. Antibodies having specific binding affinity for IL-Iβ can be produced through standard methods. Alternatively, antibodies may be commercially available, for example, from R&D Systems, Inc., Minneapolis, MN. For example, Canakinumab (Ilaris®) is an anti-IL-1β neutralizing monoclonal antibody that blocks binding to the IL-1 receptor. Gevokizumab, is a fully human monoclonal anti-human IL-1 beta antibody of the IgG2 isotype. LY 2189102 is a humanised interleukin-1 beta (IL-1β) monoclonal antibody. It will be appreciated that the IL-1β antibody and/or an anti-IL-1α antibody or fragments thereof neutralize the biological activity of IL-1β and/or an anti-IL-1 connected with the signaling function of IL-1RI. IL-1β antibodies may neutralize the biological activity of IL-1β by binding to IL-1β, and preventing the binding of the bound IL-1β to IL-1RI. The binding of IL-1β to IL-1RI may be determined by immobilizing an IL-1β binding antibody, contacting IL-1β with the immobilized antibody and determining whether the IL-1β was bound to the antibody, and contacting a soluble form of IL-1RI with the bound IL-1β/antibody complex and determining whether the soluble IL-1RI was bound to the complex. The same applies to an IL-1α antibody.

IL-1β binding antibodies may neutralize the biological activity of IL-1β by binding to IL-1β, without substantially preventing the binding of the bound IL-1β to IL-1RI. This means that they can bind and neutralize IL-1β while still permitting IL-1β to bind to IL-1RI. This can result in an effective reduction in IL-1α biological activity as well as IL-1β biological activity, since there are fewer unbound IL-1RI sites for IL-1α to bind to, thereby producing an overall decrease in IL-1RI signalling. The same applies to an IL-1α antibody.

In an embodiment, the inhibitor is an anti-IL-1R1 antibody. AMG 108, is a fully human IgG2 monoclonal antibody that binds IL-1R type 1 and non-selectively inhibits the activity of both forms of IL-1 (IL-1α and IL-1β).

Antibody fragments that have specific binding affinity for IL-1β, IL-1α or IL-1R1 can be generated by known techniques. For example, such fragments include, but are not limited to, F(ab′)2 fragments that can be produced by pepsin digestion of the antibody molecule, and Fab fragments that can be generated by reducing the disulfide bridges of F(ab′) fragments. Alternatively, Fab expression libraries can be constructed. See, for example, Huse et al. 1989, Science, 246:1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

The inhibitors of the invention or a formulation thereof may be administered by any conventional method including parenteral (e.g., subcutaneous or intravenous) injection.

The inhibitors disclosed herein or used as described herein may be administered orally, topically, parenterally, by inhalation or spray, sublingually, via implant, including ocular implant, transdermally, via buccal administration, rectally, as an ophthalmic solution, injection, including ocular injection, intravenous, intra-aortal, intracranial, subdermal, intraperitoneal, subcutaneous, transnasal, sublingual, intrathecal, or rectal or by other means, in dosage unit formulations containing conventional pharmaceutically acceptable carriers.

The pharmaceutical composition may be formulated as any pharmaceutically useful form, e.g., as an aerosol, a cream, a gel, a gel cap, a pill, a microparticle, a nanoparticle, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution or suspension. Some dosage forms, such as tablets and capsules, are subdivided into suitably sized unit doses containing appropriate quantities of the active components, e.g., an effective amount to achieve the desired purpose. An effective amount of the inhibitor as described herein may be incorporated into a nanoparticle, e.g. for convenience of delivery and/or extended release delivery.

Pharmaceutical compositions suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

Whilst it is possible for a compound of the invention to be administered alone, it is preferable to present it as a pharmaceutical composition, together with one or more acceptable excipient, diluent and/or carriers. The carrier(s) must be “acceptable” in the sense of being compatible with the compound of the invention and not deleterious to the recipients thereof. Typically, the carriers will be water or saline which will be sterile and pyrogen free. The pharmaceutical compositions contemplated here can optionally include a carrier. Carriers must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. Classes of carriers include, but are not limited to binders, buffering agents, coloring agents, diluents, disintegrants, emulsifiers, fillers, flavorants, glidents, lubricants, pH modifiers, preservatives, stabilizers, surfactants, solubilizers, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Examples of other matrix materials, fillers, or diluents include lactose, mannitol, xylitol, microcrystalline cellulose, calcium diphosphate, and starch. Examples of surface active agents include sodium lauryl sulfate and polysorbate 80. Examples of drug complexing agents or solubilizers include the polyethylene glycols, caffeine, xanthene, gentisic acid and cylodextrins. Examples of disintegrants include sodium starch gycolate, sodium alginate, carboxymethyl cellulose sodium, methyl cellulose, colloidal silicon dioxide, and croscarmellose sodium. Examples of binders include methyl cellulose, microcrystalline cellulose, starch, and gums such as guar gum, and tragacanth. Examples of lubricants include magnesium stearate and calcium stearate. Examples of pH modifiers include acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids. Optional other active agents may be included in a pharmaceutical composition, which do not substantially interfere with the activity of the compound of the present invention.

Preferably, the inhibitor or pharmaceutical composition comprising the inhibitor is administered to the subject in a therapeutically effective amount. By “therapeutically effective amount” we include the meaning of an amount of inhibitor or pharmaceutical composition comprising the inhibitor to produce the desired pharmacological effect in the subject, such as to treat the subject as described herein.

The pharmaceutical compositions may be provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The inhibitor may be formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the inhibitor sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The inhibitor and/or compositions may be formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The inhibitor provided herein can be administered at once, or may be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

The precise dose to be employed in the formulation will also depend on the route of administration, and the seriousness of the disease and/or condition, and should be decided according to the judgment of the practitioner and each patient's circumstances. Effective doses may be extrapolated from dose-response curves derived from in vitro or animal model test systems.

In some embodiments, an inhibitor may be administered via multiple routes of administration simultaneously or subsequently to other doses of the same or a different inhibitor.

For oral and parenteral administration to human patients, a daily dosage level of the compounds of the invention will usually be from about 0.015 to 25 mg/kg, such as about 5-20 mg/Kg), administered in single or divided doses. In some embodiments, the dosage administered to a patient is typically 0.1 mg/kg to 100 mg/kg of the patient's body weight. In some embodiments, the dosage administered to the patient is about 1 mg/kg to about 75 mg/kg of the patient's body weight. Preferably, the dosage administered to a patient is between 1 mg/kg and 20 mg/kg of the patient's body weight, more preferably 1 mg/kg to 5 mg/kg of the patient's body weight. However, lower dosages and less frequent administration is also possible.

The inhibitor of the invention can also be administered parenterally, for example, intravenously, intra-arterially, intraperitoneally, intrathecally, intraventricularly, intracranially, or intrasternally, intra-muscularly subcutaneously, or they may be administered by infusion techniques. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

The composition may formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lignocamne to ease pain at the site of the injection. Such compositions, however, may be administered by a route other than intravenous.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The concentration of the pharmaceutically active compound is adjusted so that an injection provides an effective amount to produce the desired pharmacological effect. The exact dose depends on the age, weight and condition of the patient or animal as is known in the art.

In an embodiment, the subject is also administered an inhibitor of VCAN.

By an “inhibitor of VCAN” we include the meaning of any molecule that inhibits, reduces, blocks and/or antagonises a biological activity of VCAN. For the avoidance of doubt, we also include any molecule that prevents or decreases the expression of VCAN and/or increases its degradation.

Inhibitors of VCAN may include TLR1/2 receptor antagonist, a VCAN siRNA, an anti-VCAN antibody, a soluble TLR1/2, monobodies and aptamers.

By “biological activity of VCAN” we include the biological action VCAN, and this refers to any function(s) exhibited or performed by a naturally occurring, and/or wild type form of VCAN as measured or observed in vivo (i.e. in the natural physiological environment of the protein) or in vitro (i.e. under laboratory conditions). Such actives include those known in the art or described herein. Methods for evaluating VCAN inhibition are known in the art and described herein.

We include the meaning that the activity of VCAN is reduced in the presence of the inhibitor, compared to the activity of VCAN in the absence of the inhibitor. Inhibition is not limited to complete inhibition of VCAN activity. In a given application, it may be that some low level of VCAN activity can be tolerated that will not have a detrimental effect on the outcome of the patient. In an embodiment, the inhibitor of VCAN reduces VCAN activity by at least 10%, such as at least 20%, 30%, 40% or 50% compared to the activity of VCAN in the absence of the inhibitor. In an embodiment, the inhibitor of VCAN reduces VCAN activity by at least 50%, such as at least 60%, 70%, 80% or 90%, such as by 95% compared to the activity of VCAN in the absence of the inhibitor.

In an embodiment, the VCAN inhibitor may be one that selectively inhibits VCAN. For example, the VCAN inhibitor may inhibit and/or decrease a biological activity of VCAN to a greater extent than it inhibits a biological activity of an unrelated protein. Preferably, the agent inhibits a biological activity of VCAN at least 5, or at least 10, or at least 50 times more than it inhibits a biological activity of another unrelated protein. More preferably, the agent inhibits a biological activity of VCAN at least 100, or at least 1,000, or at least 10,000 times more than it inhibits a biological activity of another unrelated protein.

Preferably, the VCAN inhibitor is selected from the group comprising: a small molecule, a peptide, a polypeptide, a nucleic acid molecule (such as an aptamer), an antibody, a peptidomimetic, a natural product, a monobody, or a carbohydrate.

In an embodiment, the inhibitor of VCAN is a toll-like receptor 1/2 (TLR1/2) inhibitor. As shown in the accompanying Examples, the toll-like receptor 1/2 (TLR1/2) inhibitor Cu-CPT22 (3,4,6-Trihydroxy-2-methoxy-5-oxo-5H-benzocycloheptene-8-carboxylic acid hexyl ester) blocks versican (VCAN)-induced myeloid NF-κB activation and MPE of Kras-mutant cancer cells in vivo. In an embodiment, the TLR1/2 inhibitor is Cu-CPT22. Other known inhibitors of TLR1/2 are known in the art and include NCI35676 (a natural product from nutgalls and oak barks named purpurogallin). It will be appreciated that the best strategy for targeting versican would be to focus on regions that are not isoform specific.

The subject may be administered the inhibitor of IL-1R1 signalling and a VCAN inhibitor in combination. The term “in combination with” is understood as the two or more drugs are administered subsequently or simultaneously, in other words they are not necessarily co-formulated. Alternatively, the term “in combination with” is understood that two or more drugs are administered in the manner that the effective therapeutic concentration of the drugs are expected to be overlapping for a majority of the period of time within the patient's body. The inhibitor of IL-1R1 signalling and one or more combination partner (e.g. another drug, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The drug may be administered to a patient as separate entities either simultaneously, concurrently or sequentially with no specific time limits, wherein such administration provides therapeutically effective levels of the two compounds in the body of the patient and the treatment regimen will provide beneficial effects of the drug combination in treating the conditions or disorders described herein. The latter also applies to cocktail therapy, e.g. the administration of three or more active ingredients.

In an embodiment, the subject is also administered one or more chemotherapeutic agent.

It will be appreciated that the subject may also be administered a chemotherapeutic agent that is the standard of care for the particular cancer.

Cancer chemotherapeutic agents include but are not limited to, Alkylating agents, Antimetabolites, Topoisomerase inhibitors, Mitotic inhibitors, Antitumor antibiotics, Immunotherapy drugs including checkpoint inhibitors, Receptor tyrosine kinase inhibitors, and miscellaneous agents including platinum coordination complexes such as cisplatin (cis-DDP) and carboplatin; anthracenedione (anthraquinone or dioxoanthracene) such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

In another aspect, the invention provides a method of selecting a subject that has a cancer, which cancer is predicted to respond therapeutically to a treatment comprising an inhibitor of interleukin-1 receptor type 1 (IL-1R1) signalling, comprising the steps of:
a) determining whether the cancer comprises a KRAS mutation; and
b) determining whether the cancer has an elevated level and/or activity of veriscan (VCAN);
wherein if the cancer from the subject comprises a KRAS mutation and has an elevated level and/or activity of VCAN, the subject is selected as having a cancer that is predicted to respond therapeutically to a treatment comprising an inhibitor of interleukin-1 receptor type 1 (IL-1R1) signalling.

It will be appreciated that determining whether the cancer comprises a KRAS mutation and determining whether the cancer has an elevated level and/or activity of veriscan (VCAN) may be carried out using any of the methods described herein. Preferences for the KRAS mutation and the elevated level and/or activity of veriscan (VCAN) include those described herein, as are preferences for the inhibitor of interleukin-1 receptor type 1 (IL-1R1) signalling.

In an embodiment, determining whether the cancer has an elevated level and/or activity of VCAN comprises determining whether the level and/or activity of VCAN in the cancer is elevated relative to a level and/or activity of VCAN in a reference sample.

In an embodiment, the method further comprises the step of:
c) determining whether the cancer has an elevated level and/or activity of IL-1β, wherein if the cancer from the subject comprises a KRAS mutation, an elevated level and/or activity of VCAN, and has an elevated level and/or activity of IL-1β, the subject is selected as having a cancer that is predicted to respond therapeutically to a treatment comprising an inhibitor of interleukin-1 receptor type 1 (IL-1R1) signalling.

It will be appreciated that determining whether the cancer has an elevated level and/or activity of IL-1β may be carried out using any of the methods described herein. Preferences for the elevated level and/or activity of IL-1β include those described herein.

In an embodiment, determining whether the cancer has an elevated level and/or activity of IL-1β comprises determining whether the level and/or activity of IL-1β in the cancer is elevated relative to a level and/or activity of IL-1β in a reference sample.

In an embodiment, any of steps of (a), (b) and (c) are carried out in vitro and/or on a sample provided from the subject.

Preferences for the sample include those as described above. For example the sample may be a cellular sample, a tissue sample a blood sample, a serum sample, or a sample of pleural or peritoneal fluids. Preferably, the test sample comprises cancerous cells. The sample may be a biopsy sample taken from a subject, for example, one that contains suspected or known cancer cells. The sample may be archived paraffin-embedded tumor tissue. The sample may be a tissue sample (e.g., a sample of lung, kidney, skin, thymus, breast, pancreatic, thyroid, bladder, liver, cervix, endometrium, colon, rectum and/or stomach).

In an embodiment, the method further comprises treating the subject that has been selected as having a cancer that is predicted to respond therapeutically to a treatment comprising an inhibitor of interleukin-1 receptor type 1 (IL-1R1) signalling with an inhibitor of interleukin-1 receptor type 1 (IL-1R1) signalling.

In another aspect the invention provides use of veriscan (VCAN) as a biomarker for determining whether a subject having a cancer that comprises a KRAS mutation is suitable for treatment with an inhibitor of interleukin-1 receptor type 1 (IL-1R1) signalling.

Preferences for the inhibitor of interleukin-1 receptor type 1 (IL-1R1) signalling are described herein.

As described in the accompanying Examples, VCAN is overexpressed in human KRAS-mutant cancers and can serve as a diagnostic and prognosis biomarker.

By “biomarker” we include the meaning of a naturally-occurring biological molecule, or component or fragment thereof, the measurement of which can provide information useful in the prognosis and/or diagnosis of cancer suitable for treatment with an inhibitor of interleukin-1 receptor type 1 (IL-1R1) signalling. For example, the biomarker may be a naturally-occurring protein or carbohydrate moiety, or an antigenic component or fragment thereof.

In an embodiment, the use comprises determining whether the subject having a cancer that comprises a KRAS mutation comprises an elevated level and/or activity of VCAN, as described herein. Thus, it will be appreciated that VCAN may be useful as a biomarker to identify subjects that are amenable to treatment with an inhibitor of interleukin-1 receptor type 1 (IL-1R1) signalling.

The use may comprise measuring the expression of a nucleic acid molecule encoding VCAN. Measuring the expression of VCAN can be performed using a method selected from the group consisting of Southern hybridisation, Northern hybridisation, polymerase chain reaction (PCR), reverse transcriptase PCR (RT PCR), quantitative real-time PCR (qRT-PCR), nanoarray, microarray, macroarray, autoradiography and in situ hybridisation. Alternatively or additionally the nucleic acid molecule is a ctDNA molecule, a cDNA molecule or an mRNA molecule.

The use may comprise measuring the expression of a protein molecule encoding VCAN. Measuring the expression of VCAN can be performed using any suitable method known in the art such as ELISA, western blotting, immunofluorescence, immunohistochemistry.

In an embodiment, IKKβ is also used as a biomarker for determining whether a subject having a cancer that comprises a KRAS mutation is suitable for treatment with an inhibitor of interleukin-1 receptor type 1 (IL-1R1) signalling. In an embodiment, the use comprises determining whether the subject having a cancer that comprises a KRAS mutation comprises an elevated level and/or activity of IKKβ, as described herein.

The present invention provides kits that can be used in any of the above methods.

In an aspect, the invention provides a kit comprising:
(i) a reagent for detecting the presence of a KRAS mutation; and
(ii) a reagent for determining the level and/or activity of VCAN.

It will be appreciated that such a kit may be used to identify a subject that is suitable for treatment with an inhibitor of interleukin-1 receptor type 1 (IL-1R1) signalling in the methods described herein.

By a “reagent for detecting the presence of a KRAS mutation” we include the meaning of any suitable reagent that can be used to detect the presence of a KRAS mutation in a subject or sample taken therefrom. Methods for detecting a KRAS mutation are described herein. For example, the reagent may be a mutation-specific primer that can be used in PCR, such as in QPCR.

By a “reagent for determining the level and/or activity of VCAN” we include the meaning of any suitable reagent that can be used to detect the level and/or activity of VCAN in a subject or sample taken therefrom. The reagent for determining the level and/or activity of VCAN may comprise or consist of a nucleic acid, such as primer for use in PCR, or a probe for use in immunohistochemistry. The reagent for determining the level and/or activity of VCAN may comprise or consist of an antibody or antigen-binding fragment of the same, or a variant thereof. In an alternative or additional embodiment, the reagent(s) is/are immobilised on a surface (e.g. on a multiwell plate or array). Alternatively or additionally the reagent may be labelled directly or indirectly with a detectable moiety. Suitable detectable moieties are well known in the art. For example, the antibody may be conjugated to a detectable moiety such as a fluorescent compound, an enzymatic substrate, a radioactive compound or a luminescent compound, or a second antibody which recognizes the first antibody may be conjugated to a detectable moiety). The reagent for determining the level and/or activity of VCAN could comprise or consist of an antibody or antigen-binding fragment of the same, or a variant thereof for use in an ELISA for detecting VCAN.

The detectable moiety may be a fluorescent and/or luminescent and/or chemiluminescent moiety which, when exposed to specific conditions, may be detected. For example, a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

Alternatively, the detectable moiety may be a radioactive atom which is useful in imaging. Suitable radioactive atoms include 99mTc and 123I for scintigraphic studies. Other readily detectable moieties include, for example, spin labels for magnetic resonance imaging (MRI) such as 123I again, 131I, 111In, 19F, 13C, 15N, 17O, gadolinium, manganese or iron. Clearly, the agent to be detected (such as, for example, KRAS, VCAN, IL-1β, and/or IKKβ) is in the test sample and/or control sample described herein and/or an antibody molecule for use in detecting a selected protein) must have sufficient of the appropriate atomic isotopes in order for the detectable moiety to be readily detectable.

The radio- or other labels may be incorporated into the agents of the invention (i.e. the proteins present in the samples of the methods of the invention and/or the binding agents of the invention) in known ways. For example, if the binding moiety is a polypeptide it may be biosynthesised or may be synthesised by chemical amino acid synthesis using suitable amino acid precursors involving, for example, fluorine-19 in place of hydrogen. Labels such as 99mTc, 123I, 186Rh, 188Rh and 111 In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate 123I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

The reagent for determining the level and/or activity of VCAN could be the NF-κB reporter Raw 264.7 cell line as described herein.

In an embodiment, the kit further comprises:
(iii) a reagent for determining the level and/or activity of IL-1β.

By “a reagent for determining the level and/or activity of IL-1β” we include the meaning of any suitable reagent that can be used to detect the level and/or activity of IL-1β in a subject or sample taken therefrom. The reagent for determining the level and/or activity of IL-1β may comprise or consist of a nucleic acid, such as primer for use in PCR, or a probe for use in immunohistochemistry. The reagent for determining the level and/or activity of IL-1β may comprise or consist of an antibody or antigen-binding fragment of the same, or a variant thereof. In an alternative or additional embodiment, the reagent(s) is/are immobilised on a surface (e.g. on a multiwell plate or array). Alternatively or additionally the reagent may be labelled directly or indirectly with a detectable moiety. Suitable detectable moieties are well known in the art and described herein. The reagent for determining the level and/or activity of IL-1β could comprise or consist of an antibody or antigen-binding fragment of the same, or a variant thereof for use in an ELISA for detecting IL-1β.

In an embodiment, the kit comprises a reagent for determining the level and/or activity of IKKβ. By “a reagent for determining the level and/or activity of IKKβ” we include the meaning of any suitable reagent that can be used to detect the level and/or activity of IKKβ in a subject or sample taken therefrom. The reagent for determining the level and/or activity of IKKβ may comprise or consist of a nucleic acid, such as primer for use in PCR, or a probe for use in immunohistochemistry. The reagent for determining the level and/or activity of IKKβ may comprise or consist of an antibody or antigen-binding fragment of the same, or a variant thereof. Such a reagent includes any such reagent described in the methods for determining the level and/or activity of IKKβ described herein.

The invention also provides the inhibitor for use, a use, method, or kit substantially as described herein by reference to the accompanying description and/or drawings.

Preferred, non-limiting examples which embody certain aspects of the invention will now be described, with reference to the following tables and figures:

### FIGURES

FIG. 1. Non-oncogene addiction of KRAS-mutant cancers to interleukin (IL)-1B. (A, B) TP53, KRAS, EGFR, and BRAF mutation frequencies in the canakinumab anti-inflammatory thrombosis outcomes study (CANTOS) and the cancer genome atlas (TCGA) lung adenocarcinoma (LUAD) patients. Data from (2, 12), https://www.cbioportal.org/, and https://bit.ly/3uSO0D4. Shown are patient and mutation numbers (n) and percentages (%), as well as probabilities (P), χ² test (A) or hypergeometric test (B). (C) Data summary of KRAS alterations versus IL1B mRNA expression in the cancer genome atlas (TCGA) pan-cancer dataset (n=10,967 samples from 10,953 patients with 31 different cancers) from the US. Data from https://www.cbioportal.org/and https://bit.ly/3c1KOyl. RSEM, RNA-Seq by Expectation-Maximization. Note the elevated IL1B mRNA expression of KRAS-altered cancers. (D) Data summary (left) and representative images (right; inlays: isotype controls) of KRAS alterations versus IL-1β protein expression in lung adenocarcinoma (LUAD) and adjacent lung tissue from n=36 resected patients from Munich, Germany. Note the elevated IL-1β protein expression of KRAS-altered LUAD. (E) Data summary of subcutaneous (s.c.) tumor and malignant pleural effusion (MPE) volume of C57BL/6 mice competent (WT) or diploinsufficient (Il1b−/−) in Il1b alleles at the indicated time-points after s.c. or intrapleural injection of 5 or 2×105 tumor cells, respectively, with (left; n from left to right=10, 10, 30, 30, 10, 10, 20, and 20) or without (right; n=10/group) Kras mutations. Note the requirement of Kras-altered tumors for host IL-1β. Shown are raw data (circles), rotated kernel density distributions (violins), medians (dashed lines), quartiles (dotted lines), and P, probabilities, Kolmogorov-Smirnov or Kruskal-Wallis test (A), two-way ANOVA (B, above graph) and Bonferroni post-tests (B, in graph), or unpaired tests (C). *** and ****: P<0.001 and P<0.0001, respectively, compared with diploid patients, Dunn's post-tests.

FIG. 2. Kras-mutant tumors activate NF-κB in tumor-infiltrating macrophages. (A) Color coded cancer cell lines used with tissues of origin and Kras mutation status. (B-E) Bioluminescent images with pseudocolor scales (B, C) and data summaries (D, E) from WT and HIV-LTR. Luciferase (HLL: B and D), and NF-κB.GFP.Luciferase (NGL; C and E) NF-κB reporter mice at 14 days (B, D) or serial time-points (C, E) post-pleural injection of tumor cells. Note that in these models bioluminescence is exclusively emitted by host and not tumor cells. (B, C) Dashed areas delineate the thorax. (F) Photographic/biofluorescent image overlay with pseudocolor scale of NGL mouse lung explant 14 days post-pleural LLC cells shows NF-κB reporter GFP signal (KB.eGFP) over pleural tumors (outlines; n=10). (G) GFP immunoreactivity of pleural tumor sections co-localizes with the macrophage marker CD68 (arrows; n=10). (H, I) Flow cytometric contour plots (H) and data summary (I) of pleural tumor cells from NGL mice obtained 14 days post-pleural injection stained for the myeloid marker CD11b and the κB.LUC reporter. Percentages in (H) pertain to CD11b+LUC+ cells. Data in (D, E, I) are given as raw data (circles), median (dashed lines), quartiles (dotted lines), and kernel density distributions (violin plots) color-coded as in (A). Sample size (n)=5-10/group; P, probability, one- or two-way ANOVA; *, **, ***, and ****, P<0.05, P<0.01, P<0.001, and P<0.0001, respectively, compared with mice injected with RAW264.7, PANO2, or B16F10 cells at the same time-points, Bonferroni post-tests.

FIG. 3. Tumor-secreted factors drive IKKβ activation, differentiation, and IL-1β secretion in macrophages. (A) Color-coded cancer cells with Kras mutation status. (B-E) Bioluminescent images with pseudocolor scale (B, E), immunoblots (C), and data summaries (D, E) from exposure of RAW264.7 macrophages stably expressing pNGL (B-D) and murine bone marrow derived macrophages (BMDM) obtained from NGL mice after one-week 100 ng/ml M-CSF exposure (E) to cell-free tumor-conditioned media or DMEM (white boxes) with or without bortezomib pre-treatment (1 μg/mL˜3 UM for 1 hour). (D, E) n=5/group; P, probability, one or two-way ANOVA; **** and ####, P<0.0001 compared with other groups or saline-treated cells, respectively, Bonferroni post-tests. (F) Flow cytometry-assessed differentiation marker expression of murine bone marrow cells before (day 0) and after (day 7) one-week M-CSF exposure. (G) Microarray strategy and top-five differentially expressed genes (ΔGE) of murine BMDM compared with cancer cells (ΔGE1) and of tumor-conditioned BMDM compared with naïve BMDM (ΔGE2). n=5/group; P, probability, one-way ANOVA. (H) Bioluminescent images and data summary of NGL, NGL; Tnf−/−, and NGL;Il1b−/− mice 14 days post-pleural injection of LLC cells. n=13/group; P, probability, one-way ANOVA; ** and ***, P<0.01 and P<0.001, respectively, compared with NGL mice, Bonferroni post-tests. (I-L) Histograms (I) and data summaries (J-L) of naïve or tumor-conditioned BMDM for macrophage differentiation markers (I, J), Tnf and Il1b mRNA (K), and IL-1β protein (L) expression. n=5-10/group; P, probability, one-way ANOVA; *** and ****, P<0.001 and P<0.0001, respectively, compared with DMEM and B16F10-conditioned media, Bonferroni post-tests. Data are given as raw data (circles), medians (dashed lines), quartiles (dotted lines), and kernel density distributions (violin plots) color-coded as in (A).

FIG. 4. Tumor-secreted versican drives macrophage IKKβ, metastasis, and is a cancer biomarker. (A-E) Kras-mutant and wild-type cancer cell RNA and supernatants were subjected to microarray and LC-MSMS analyses, respectively. Shown are experimental design (A), top-20 over-represented transcripts (B) and secretory proteins (C), and Vcan/VCAN mRNA/protein expression (D, E). n=2-3/group; P, probability, two-way ANOVA; Bold letters, false discovery rate (FDR) q<0.05 compared with Kras-wild-type cells, two-stage linear step-up procedure of Benjamini, Hochberg, and Yekutieli. (F, G) Representative bioluminescent images (F) and data summary (G) from pNGL RAW264.7 cells exposed to lipopolysaccharide (LPS; 1 μg/mL) or recombinant proteins (1-2 nM). n=5/group; P, probability, two-way ANOVA; ****, P<0.0001 compared with other groups, Bonferroni post-tests. (H) Immunoblots of mouse BMDM exposed to VCAN. n=5/group. (I-M) LLC cells stably expressing control (shC) and anti-Vcan (shVcan) shRNA were validated and injected intrapleurally into NGL mice. Shown are immunoblots (I), Vcan mRNA expression (J), and data summaries (K, L) and representative photographic and bioluminescent images (M) taken 14 days post-tumor cells. (I, J) n=5/group; (K-M) n=16/group; P, probability, unpaired Student's t-test. (N) Images and data summary of VCAN expression of n=41 tumor/normal tissue pairs from patients with resected lung adenocarcinoma. (O) VCAN mRNA expression of 10 benign and 15 malignant pleural effusions. (P) Data summary and representative image of bioluminescence of pNGL RAW264.7 cells after exposure to benign (n=6; top triplicates) and malignant (n=11; bottom triplicates) pleural effusions and tumor-conditioned media (each triplicate column is one patient). (N-P) P, probability, unpaired Student's t-test. (B-D, G, J-L, N-P) Shown are raw data (circles), kernel density distributions (violins), and medians/quartiles (dashed/dotted lines).

FIG. 5. IKKβ mediates pro-tumor NF-κB activity, differentiation, and IL-1β secretion in macrophages. (A, B) Bioluminescent image with pseudocolor scale (A) and data summary (B) of pNGL RAW264.7 cells 72 hours post-infection with control (shC), anti-GFP (shGFP), or anti-inhibitor of NF-κB kinase (shChuk, shIkbkb, shIkbke, or shTbk1)-specific shRNAs. n=8 independent experiments/group; P, probability, one-way ANOVA; ****, P<0.0001 compared with shC, Bonferroni post-tests. (C-F) Bone marrow-derived macrophages (BMDM) were derived from mT/mG;Lyz2.Cre, Chukf/f;Lyz2.Cre, and Ikbkbf/f;Lyz2.Cre mice using one-week exposure to 100 ng/ml M-CSF. Shown are images and mean±SD % green cells of bone marrow cells from mT/mG;Lyz2.Cre mice during/after weekly treatment with M-CSF (C), flow cytometric histograms (left) and data summary (right) of marker expression (D), top differentially expressed genes by microarray (E), and interleukin (IL)-1β secretion by ELISA (F). (C, D) n=5 independent experiments/group; P, probability, Fisher's exact test or one-way ANOVA; **, P<0.01 compared with other groups, Bonferroni post-tests. (E) n=1 pooled triplicate/group; P, probabilities, two-way ANOVA. (F) n=10 independent experiments; P, probability, one-way ANOVA; ** and ***, P<0.01 and P<0.001, with respectively, compared controls, Bonferroni post-tests. (G) Chukf/f; Lyz2.Cre and Ikbkbf/f; Lyz2. Cre mice received intrapleural LLC or MC38 cells, and were evaluated after 14 days for malignant pleural effusions (MPE). Data summary of n=40, 15, and 21 single transgenic control, Chukf/f;Lyz2.Cre, and Ikbkbf/f;Lyz2.Cre mice injected with LLC cells, respectively, and of n=40, 15, and 25 respective mice injected with MC38 cells. P, probability, two-way ANOVA; *, **, and ****, P<0.05, P<0.01, and P<0.0001, respectively, compared with controls, Bonferroni post-tests. (H) Schematic of the proposed mechanism for non-oncogene addiction of KRAS-mutant cancers to IL-1β. To this end, KRAS-mutant cancers secrete VCAN to co-opt IKKβ in macrophages within the metastatic niche, which drives IL-1β secretion by macrophages to foster tumor progression.

FIG. 6. Pharmacologic abolition of non-oncogene addiction of Kras-mutant tumors to IL-1β. (A-D) The IL-1 receptor antagonist Isunakinra limits nuclear factor (NF)-κB activation and malignant pleural effusions (MPE) from Kras-mutant cancer cells. (A) FVB (n=10) and C57BL/6 (n=45) mice received subcutaneous injections of 5×10⁶ FULA1 (FVB mice) or LLC, MC38, B16F10, or PANO2 (C57BL/6 mice) cells that carry G12C, G13R, Q61R, or wild-type (WT) Kras alleles. Mice were allowed 10-17 days for tumor take, and were treated with daily intraperitoneal phosphate buffered saline (PBS) or 20 mg/Kg (LLC cells) or 50 mg/Kg (all other cells) Isunakinra until control tumor volume reached 1 cm3 (PANO2 cells) or 2 cm3 (all other cell lines). Shown are therapy (Tx) start, mouse numbers (n), tumor volume as mean (circles) and SD (bars), two-way ANOVA probability (P) for treatment effects, and average Isunakinra effect at the last time-points (%). ** and ***: P<0.01 and P<0.001, respectively, Bonferroni post-tests. (B-D) C57BL/6 mice (n=48) received intrapleural 2×105 LLC cells stably expressing a κB.LUC reporter (NGL), were allowed five days for tumor take, and received daily intraperitoneal PBS or 20 mg/Kg Isunakinra. Mice were sacrificed when morbid (n=17/treatment) or at day 14 post-LLC cells (n=7/treatment), after 1 mg intravenous D-luciferin and bioluminescence imaging. Shown are representative chest (dotted lines) bioluminescent images with pseudocolor scale (B), Kaplan-Meier survival estimates (curves) with log-rank probability (P) and hazard ratio (HR) (C), and data summary of chest bioluminescence and MPE volume (H), shown as raw data points (circles), medians (dashed lines), quartiles (dotted lines), kernel density distributions (violins), and probability (P), unpaired Student's t-test. (E-H) The toll-like receptor 1/2 (TLR1/2) inhibitor Cu-CPT22 blocks versican (VCAN)-induced myeloid NF-κB activation and MPE of Kras-mutant cancer cells in vivo. (E, F) Representative bioluminescent image with pseudocolor scale (E) and results summary (F) of RAW264.7 macrophages stably expressing NGL that were pre-treated with 1% DMSO or increasing Cu-CPT22 concentrations in 1% DMSO and were exposed (1 hour latency) to 10 nM lipopolysaccharide (LPS) or 1 nM recombinant VCAN. Cells were assessed for bioluminescence at 24 and MTT reduction at 72 hours post-LPS/VCAN treatments. n=3 and n=6 independent experiments/group for κB.LUC and MTT, respectively, are shown as 50% inhibitory/lethal concentrations (IC50/LC50), mean (circles), and SD (bars). (G, H) Representative images (G) and data summary (H) of chest bioluminescence and MPE volume of κB.luc mice at 14 days post-pleural injection of 2×105 LLC cells followed by treatment with daily intraperitoneal injections of 100 μl corn oil 10% DMSO (n=10) or 20 mg/kg Cu-CPT22 diluted in 100 μl corn oil 10% DMSO (n=10) initiated five days post-LLC cells. Shown are raw data points (circles), medians (dashed lines), quartiles (dotted lines), kernel density distributions (violins), and probability (P), unpaired Student's t-test.

FIG. 7 (FIG. S1) Seven murine cell lines with different Kras alleles and transcriptional control of interleukin (IL)-1B by nuclear factor (NF)-κB. (A, B) Sanger sequencing traces for Kras codons 12, 13, and 61 of all cell lines used. Arrows point to Kras mutations: MC38 cells carry G13R, LLC and AE17 cells G12C, FULA1 cells Q61R (B), and all other cell lines wild-type (Wt/WT; A) Kras alleles. Note that all mutant Kras alleles are in heterozygosity to Wt alleles. (C) IL1B as a NF-κB target gene in ChIPseq datasets from the CHEA Transcription Factor Targets dataset (https://maayanlab.cloud/Harmonizome/dataset/CHEA+Transcription+Factor+Targets). We downloaded the RELA (red) and RELB (blue) binding sequence motifs from the ENCODE portal (https://www.encodeproject.org/) with the identifiers: ENCFF507YCV (CHIP-seq on HuH-7.5 cells) and ENCFF615HZF (CHIP-seq on 8988T cells), respectively. E-value represents the statistical significance of the motif in terms of probability to be found in similarly sized set of random sequences. TSS: Transcription Start Site.

FIG. 8 (FIG. S2). Expression of IL1B in correlation with KRAS and the macrophage marker ADGRE1 in human tumors. Gene expression data from the cancer genome atlas (TCGA) pan-cancer dataset (n=10,071 patients). Correlations of IL1B with KRAS (A) and the macrophage marker ADGRE1 (encoding F4/80) (B) mRNA levels. Shown are raw data points (circles) color-coded by KRAS alteration status, regression lines and formulas (lines), as well as Spearman's and Pearson's correlation coefficients with probabilities (P) and squared correlation coefficients (R2). Data from https://www.cbioportal.org/.

FIG. 9 (FIG. S3). Expression of IL1B in correlation with lineage-specific markers in human tumors. Gene expression data from the cancer genome atlas (TCGA) pan-cancer dataset (n=10,071 patients). Correlations between mRNA levels of IL1B and the neutrophil marker ELANE (top left; encoding neutrophil elastase), the pan-lymphocyte marker CD3D (top middle; encoding cluster of differentiation 3), the mast cell marker KIT (top right; encoding c-KIT), the cancer cell marker KRT18 (bottom left; encoding cytokeratin 18), the fibroblast marker ACTA2 (bottom middle; encoding α-smooth muscle actin), and the endothelial marker F8 (bottom right; encoding factor VIII). Shown are raw data points (circles) color-coded by KRAS alteration status, regression lines and formulas (lines), as well as Spearman's and Pearson's correlation coefficients with probabilities (P) and squared correlation coefficients (R2). Data from https://www.cbioportal.org/.

FIG. 10 (FIG. S4). NF-κB activation in pleural metastases of NGL mice. (A, B) Representative photographic/bioluminescent images with pseudocolor scale (A) and data summary (B) from wild-type (WT/WT), heterozygote (WT/NGL), and homozygote (NGL/NGL) NF-κB.eGFP.LUC reporter mice at 5 min post-retroorbital injection of 1 mg D-Luciferin. Sample size (n)=10/group; P, probability, one-way ANOVA; ****, P<0.0001 compared with WT/WT mice, Bonferroni post-test. (C, D) Representative photographic/biofluorescent (C) images with pseudocolor scale and data summary (D) of KB.eGFP reporter signal (green) of lung explants of NGL mice at 14 days post-pleural injection of 2×105 Lewis lung carcinoma (LLC) cells. Note the NF-κB reporter signal (KB.eGFP) over pleural tumors (outlines). n=10/group; P, probability, unpaired Student's t-test. (E, F) Representative photographic/bioluminescent image overlays with pseudocolor scale (E) and results summary (F) of chest bioluminescence of NGL mice with metastatic malignant pleural effusions at 14 days post-pleural injection of 2×10⁵ B16F10 skin melanoma, MC38 colon adenocarcinoma, or LLC cells before (undrained) and after (drained) pleural catheter insertion and fluid removal. n=5/group; P, probability, repeated measures two-way ANOVA; ns and *, P>0.05 and P<0.05, respectively, for pre-post drainage comparison. Data in (B,D,F) are given as raw data (circles), median and quartiles (lines), and kernel density distributions (violin plots).

FIG. 11 (FIG. S5). NF-κB activation in pleural metastases of NGL mice. Pleural metastases from mice treated as in FIG. 10 C, D (FIG. S4C, D) show endogenous KB.eGFP reporter signal that co-localizes with the panhematopoietic marker CD45 (A) and the macrophage marker CD68 (B) in tumor-infiltrating myeloid cells and macrophages (arrows). Nuclear Hoechst 33258 counterstaining.

FIG. 12 (FIG. S6). NF-κB activation in metastasis-associated macrophages. NGL NF-κB reporter mice received pleural PBS or DMEM (controls), or 2×10⁵ B16F10 skin melanoma, MC38 colon adenocarcinoma, or Lewis lung carcinoma (LLC) cells and were sacrificed 14 days thereafter. Pleural fluid and pleural tumor cells were stained with antibodies against the hematopoietic marker CD45, myeloid markers CD11b and Ly6c, and the endogenous κB.LUC reporter. Representative flow cytometric dotplots showing the sequential gating strategy for macrophages of pleural fluid (A) and of tumors (B), and histogram (C) and data summary (D) of κB.LUC reporter signal in pleural macrophages. Data in (D) are given as raw data (circles), median and quartiles (lines), and kernel density distributions (violin plots). Sample size (n)=5/group; P, probability, one-way ANOVA; ****, P<0.0001 compared with mice injected with B16F10 cells, Bonferroni post-tests. MFI, mean fluorescence intensity.

FIG. 13 (FIG. S7). NF-κB activation in metastasis-associated macrophages. NGL NF-κB reporter mice received 2×10⁵ pleural B16F10 skin melanoma, MC38 colon adenocarcinoma, or Lewis lung carcinoma (LLC) cells and were sacrificed 14 days thereafter. Pleural tumors were assessed for KB.eGFP reporter signal by fluorescent microscopy after nuclear counterstaining with Hoechst 33258. Data summary (A) and representative merged microscopy images (B-D) of κB.eGFP+ pleural tumor cells. Data in (A) are given as raw data (circles), median and quartiles (lines), and kernel density distributions (violin plots). Sample size (n)=10/group; P, probability, one-way ANOVA; ****, P<0.0001 compared with mice injected with B16F10 cells, Bonferroni post tests. hpf, high power field.

FIG. 14 (FIG. S8). No impact of mast cells on the host NF-κB response to pleural metastasis and decreased intensity of the host NF-κB response to heterotopic tumor growth. (A-B) Representative photographic/bioluminescent image overlays with pseudocolor scale (A) and summary of results (B) from NGL mice additionally carrying no (NGL) or one (NGL;Cpa3.Cre) Cpa3.Cre allele that renders them, respectively, mast cell-competent or -deficient. Images and data were obtained at 14 days post-pleural injection of 2×10⁵ Lewis lung carcinoma (LLC) cells, 5 min postretroorbital injection of 1 mg D-Luciferin. Sample size (n)=8/group; P, probability, unpaired Student's t-test. (C-D) Representative photographic/bioluminescent image overlays with pseudocolor scale (C) and summary of results (D) from NGL mice at 21 days post-subcutaneous injection of 5×105 B16F10 skin melanoma, MC38 colon adenocarcinoma, or LLC cells, 5 min post-retroorbital injection of 1 mg D-Luciferin. n=12/group; P, probability, one-way ANOVA; ****, P<0.0001 compared with B16F10 cells, Bonferroni post-tests. Data in (B, D) are given as raw data (circles), median and quartiles (lines), and kernel density distributions (violin plots).

FIG. 15 (FIG. S9). Requirement for mutant Kras signaling for host NF-κB activation during pleural metastasis. (A) Tumor cells with Kras mutations (MC38 and LLC cells) were engineered to stably express shRNA encoding random control (shC) or anti-Kras-specific (shKras) sequences. Kras-wild-type RAW264.7 myelomonocytic leukaemia cells served as controls. Shown are color-coded tumor cell lines used with shRNA expression and Kras mutation status. (B, C) Tumor cells were injected into the pleural space of NGL mice (5×10⁵/mouse). (D, E) RAW264.7 macrophages expressing pNGL plasmid were pre-treated with saline or the proteasome and NF-κB inhibitor bortezomib (1 μg/mL equivalent to 3 μM), and were subsequently incubated with tumor-conditioned media (1:1 dilution in DMEM). Shown are representative photographic/bioluminescent images with pseudocolor scale (B, D) and results summaries of chest bioluminescence of NGL mice at 14 days post-tumor cells (C), and of cellular bioluminescence of pNGL RAW264.7 macrophages at 4 hours post-tumor-conditioned media (E). (C) n=10/group; (E) n=5 independent experiments/group; P, probability, two-way ANOVA; ** and ***, P<0.01 and P<0.001, respectively, compared with shC; #, P<0.05 compared with the respective saline-pre-treated cells. Data in (C, E) are given as raw data (circles), median and quartiles (lines), and kernel density distributions (violin plots).

FIG. 16 (FIG. S10). Adoptive bone marrow transplants determine host NF-κB response to pleural metastasis. NF-κB.eGFP.LUC reporter (NGL) and wild-type (WT) recipients received total-body irradiation (1100 Rad) followed by adoptive bone marrow replacement (BMT) from WT and NGL donors. After one month required for bone marrow chimerism, 500 μg liposomal clodronate was administered intrapleurally. After yet another month required for replacement of pleural myeloid cells by transplanted bone marrow cells, mice received 2×10⁵ intrapleural LLC cells and were imaged for bioluminescence after 14 days. Shown are experimental schematic (each box represents one postnatal month) (A, B), color-coded table with experimental groups and sample size (n) (C), results summary (D), and representative photographic/bioluminescent images with pseudocolor scale taken at 5 min post-retroorbital injection of 1 mg D-Luciferin (E). Data in (D) are given as raw data (circles), median and quartiles (lines), kernel density distributions (violin plots), and probability values (P), two-way ANOVA. ** and ***, P<0.01 and P<0.001, respectively, compared with mice that received BMT from NGL donors, Bonferroni post-tests.

FIG. 17 (FIG. S11). Pharmacologic and genetic macrophage ablation abolishes pleural metastasis. (A) C57BL/6 mice (n=5 mice/treatment/time-point) received intrapleural liposomal clodronate or empty liposomes (500 μg/mouse) and were subjected to pleural lavage at different time-points postclodronate for analysis of macrophage numbers expressing F4/80 (encoded by Adgre1, the mouse orthologue of human ADGRE1 from FIG. S1). P, probability, two-way ANOVA; *** and ****, P<0.001 and P<0.0001, respectively, compared with empty liposomes, Bonferroni posttest. (B) Pleural fluid volume of C57BL/6 mice at 15 days post-intrapleural delivery of 500 μg liposomal clodronate or empty liposomes followed by 2×105 intrapleural MC38 or LLC cells one day thereafter and 500 μg liposomal clodronate or empty liposome re-administration into the pleural space 3 days later. n=5 mice/group; P, probability, two-way ANOVA; * and **, P<0.05 and P<0.01, respectively, compared with empty liposomes, Bonferroni post-tests. (C, D) Pleural fluid nucleated cell number (C) and malignant pleural effusion volume (D) of C57BL/6 mice carrying either one or both Lyz2.Cre and Dta alleles, at 14 days post-intrapleural delivery of 2×105 MC38 or LLC cells. n=22, 14, 16, and 7 mice/group from left to right; P, probability, two-way ANOVA; * and **, P<0.05 and P<0.01, respectively, for Lyz2.Cre; Dta macrophage deficient mice compared with single transgenic controls, Bonferroni post-tests. All data are shown as raw data (circles), median and quartiles (lines), and kernel density distributions (violin plots).

FIG. 18 (FIG. S12). Uncropped immunoblots. Shown are uncropped immunoblots with areas displayed in main figures (dashed rectangles). (A-C) Blots shown in FIG. 3C including IKKα (A), IKKβ (B), and β-actin (C). (D, E) Inverted blots shown in FIG. 4E including versican (VCAN; D) and α-tubulin (TUBA; E). (F-H) Inverted blots shown in FIG. 4H including IKKα (H), IKKβ (F), and β-actin (G). (I, J) Inverted blots shown in FIG. 4I including VCAN (I) and TUBA (J).

FIG. 19 (FIG. S13). Toll-like receptor (TLR) expression by murine bone marrow-derived macrophages (BMDM) by microarray. Murine BMDM from C57BL/6 mice were isolated from whole bone marrow cells using one-week exposure to 20 ng/ml macrophage colony-stimulating factor (MCSF). Total cellular RNA was extracted from BMDM under various conditions, as well as from different cancer cell lines (n=9/group) and was hybridized to Affymetrix Mo Gene ST2.0 microarrays (data from GEO datasets GSE94847, GSE94880, GSE130624, and GSE130716). Shown are unsupervised clustering by all differentially expressed genes (A) and by TLR genes (B), as well as results summary for TLR gene expression (C). Data in (C) are shown as mean with SD; P, probability, two-way ANOVA; * and ****, P<0.05 and P<0.0001, respectively, compared with cancer cells, Bonferroni post-tests. Microarrays compared were GSM3744950, GSM3744952, GSM3744954, GSM3744955, GSM3744958, GSM3744961, GSM3744962, GSM3744957, and GSM3744960 (cancer cells) versus GSM2486425, GSM2486426, GSM2487750, GSM2487751, GSM3752396, GSM3752397, GSM3752398, GSM3752399, and GSM3752400 (BMDM).

FIG. 20 (FIG. S14). Versican as a potential diagnostic and prognostic biomarker of KRAS-mutant human cancers. (A, B) Data summary of VCAN expression normalized by ACTB transcripts in various human cancer types with different KRAS mutation frequencies (KRASMUT %; data from COSMIC; https://cancer.sanger.ac.uk/cosmic). (A) VCAN/ACTB expression in GEO dataset GSE43458 that encompasses lung adenocarcinomas (LUAD) from smokers (KRASMUT %=36.5%) and never-smokers (KRASMUT %=11.8%), as well as normal lung tissues from never-smokers (KRASMUT %<1.0%). (B) VCAN/ACTB expression in GEO dataset GSE103512 that encompasses prostate (KRASMUT %=2.8%), colorectal (KRASMUT %=32.4%), and lung (KRASMUT %=14.9%) cancers. (A, B) n, sample size; P, probability, one-way ANOVA; *** and ****, P<0.001 and P<0.0001, respectively, compared with lung tissue from never-smokers (A) or prostate cancer (B); #, P<0.05 compared with LUAD from never-smokers, Bonferroni post-tests. (C) Kaplan-Meier survival analyses from all patients within the KMplot database stratified by VCAN transcript expression by optimal cut-offs (data from KMplot; http://kmplot.com/analysis/index.php?p=service&cancer=pancancer_rnaseq. Shown is summary of hazard ratios (HR) obtained for VCAN and ACTB control expression for 7,462 patients with 18 different tumor types. P, probability, one-sample Wilcoxon test for comparison with HR=1.0. All data are shown as raw data (circles), median and quartiles (lines), and kernel density distributions (violin plots).

FIG. 21 (FIG. S15). Versican over-expression by KRAS-mutant human cancers predicts poor survival. Kaplan-Meier survival plots with median overall survival (OS), hazard ratios (HR) with 95% confidence interval, and univariate log-rank probability values (P) from 504 patients with lung adenocarcinoma (A), 373 with ovarian cancer (B), 371 with stomach cancer (C), 177 with pancreatic cancer (D), 370 with liver cancer (E), and 304 with cervical cancer (F), stratified into low (black) and high VCAN transcript expression by the optimal cut-offs indicated. RMA, robust microarray average (data from KMplot; http://kmplot.com/analysis/index.php?p=service&cancer=pancancer_rnaseg)

FIG. 22 (FIG. S16). KRAS, VCAN, and IKBKB alterations in human cancers. KRAS, VCAN, and IKBKB mutation frequencies in the cancer genome atlas (TCGA) pan-cancer dataset (n=10,967 samples from 10,953 patients). Data are from https://www.cbioportal.org/(link: https://bit.ly/3yGys8i). Shown are mutation plot with alteration frequencies (A), lollipop plots (B), co-occurrence Venn diagram (C), and most frequently altered tumor types (D). In (A), columns represent patients and rows genes. In (C), shown are sample numbers (n). P, probability, hypergeometric test. In (D), shown are raw data points (circles), rotated kernel density distributions (violins), medians (dashed lines), quartiles (dotted lines), top quartile-altered cancer types with P, two-way ANOVA, and Spearman's correlation coefficients (p) and P across 32 cancer types. TCGA acronyms are from https://gdc.cancer.gov/resources-tcga-users/tcga-code-tables/tcga-study-abbreviations. Note the alteration enrichment (addiction) of VCAN to KRAS and of IKBKB to VCAN along the pathway proposed here. Note also that lung adenocarcinoma (LUAD), colon adenocarcinoma (COAD), and uterine corpus endometrial carcinoma (UCEC) are among the top 25% mutated cancers for all three genes and among the most frequent cancer types to metastasize to the pleural space.

FIG. 23 (FIG. S17). VCAN and IKBKB alterations in lung adenocarcinoma (LUAD), colon adenocarcinoma (COAD), rectal adenocarcinoma (READ), and uterine corpus endometrial carcinoma (UCEC). KRAS, VCAN, and IKBKB mutation frequencies in the cancer genome atlas (TCGA) LUAD, COAD, READ, and UCEC datasets (n=1,689 samples/patients). Data are from https://www.cbioportal.org/and can be retrieved at https://bit.ly/3wzrbFF. Shown are mutation plot with alteration frequencies (A), co-occurrence Venn diagram (B), and features of altered versus unaltered patients (C). In (A), columns represent patients and rows genes. In (B), shown are sample numbers (n). P, probability, hypergeometric test. In (C), shown are rotated kernel density distributions (violins), medians (dashed lines), quartiles (dotted lines), and UCEC patient numbers (n) and percentages (%). TCGA acronyms are from https://gdc.cancer.gov/resourcestcga-users/tcga-code-tables/tcga-study-abbreviations. P, probability, Kruskal-Wallis test (graphs) or overall and paired χ² tests for patient numbers and percentages before and in parentheses (table). *, ***, and ****: P<0.05, P<0.001, and P<0.0001 compared with none-altered patients, Dunn's post-tests. In (B), note the alteration enrichment (oncogene addiction) of IKBKB to VCAN and the oncogene repulsion of IKBKB to KRAS. Note also the significant enrichment of VCAN and IKBKB alterations in the selected highly KRAS-mutant tumor types. In (C), note that VCAN-, IKBKB-, and VCAN/IKBKB-altered patients display varying degrees of cachexia (not recorded for LUAD), hypermutation, microsatellite instability (MSI), hypoxia, and advanced tumor grade.

### EXAMPLE 1—NON-ONCOGENE ADDICTION OF KRAS-MUTANT CANCERS TO IL-1β VIA VERISCAN AND MONONUCLEAR IKKβ

KRAS-mutant cancers are frequent, metastatic, lethal, and largely undruggable. While interleukin (IL)-1B and nuclear factor (NF)-κB inhibition hold promise against cancer, untargeted treatments are not effective. Here we show that human KRAS-mutant cancers are addicted to IL-1β via inflammatory versican signaling to macrophage inhibitor of NF-κB kinase (IKK) B.

Human pan-cancer and experimental NF-κB reporter, transcriptome, and proteome screens reveal that KRAS-mutant tumors trigger macrophage IKKβ activation and IL-1β release via secretory versican. Tumor-specific versican silencing and macrophage-restricted IKKβ deletion prevents myeloid NF-κB activation and metastasis. Versican and IKKβ are mutually addicted and/or overexpressed in human cancers and possess diagnostic and prognostic power. Non-oncogene KRAS/IL-1β addiction is abolished by IL-1β and TLR1/2 inhibition, indicating cardinal and actionable roles for versican and IKKβ in metastasis.

Tumor-associated inflammation is intimately linked with tumor progression and therapy response (1). Interleukin (IL)-1β is an important mediator of tumor-associated inflammation and its inhibition via the monoclonal antibody canakinumab was recently shown to possess strong protective effects against incident lung cancer in an exploratory analysis of the canakinumab anti-inflammatory thrombosis outcomes study (CANTOS) (2). Unexpectedly, the phase III CANOPY-2 trial (ClinicalTrials.gov NCT03626545) investigating second/third-line canakinumab with docetaxel against non-small cell lung cancer (NSCLC) irrespective of histologic subtype and driver mutation was negative for unknown reasons (https://www.novartis.com/news/media-releases/novartis-provides-update-phase-iii-studyevaluating-canakinumab-acz885-second-or-third-line-treatment-combination-chemotherapy-nonsmall-cell-lung-cancer). To this end, the protective effects of canakinumab in the CANTOS trial NSCLC exploratory study were significantly stronger for current and former smokers and for incipient lung adenocarcinoma (LUAD) histologic subtype, with both carrying high mutation rates of the KRAS proto-oncogene GTPase (encoded by the KRAS/Kras genes in humans/mice).

Multiple lines of evidence dictate that tumor genomic alterations largely define tumor-associated inflammation and the efficacy of immune-directed therapies (1). To this end, NSCLC with high mutation burden and a smoking-associated trinucleotide signature were found to display more favorable and durable responses to the immune checkpoint inhibitor pembrolizumab targeting programmed cell death-1 (PD-1) (3). Moreover, STK11/LKB1 alterations were reported to be cardinal drivers of primary resistance to PD-1 inhibitors in KRAS-mutant LUAD, the most frequent and lethal histologic subtype of NSCLC (4). Experimental evidence supports that the immune landscape and vulnerabilities of various tumor types can rely on single mutated driver oncogenes such as KRAS and MYC that orchestrate distinct transcriptional programs, dictate a tumor's specific pro-inflammatory mediator secretory profile, and largely define the cellular composition of the tumor microenvironment (5, 6). In this regard, oncogenic KRAS is known to cooperate with pro-inflammatory nuclear factor (NF)-κB signaling in cancer cells to drive stemness, pro-inflammatory mediator elaboration, and responsiveness to IL-1β signaling (7-10), and is ideally positioned as a biomarker of therapeutic response to anti-IL-1β therapy.

Here we show that KRAS-mutant cancers display specific non-oncogene addiction to host provided IL-1β in humans and mice. We further elucidate how these tumors activate NF-κB in tumor-associated macrophages in order to elicit the IL-1β they require for sustained growth. Mutant KRAS-IL-1β addiction is mediated via secretion of the glycoprotein versican (VCAN) by tumor cells, which induces inhibitor of NF-κB kinase (IKK) β in macrophages resulting in IL-1β release into the tumor microenvironment. Importantly, the VCAN-IKKβ axis is shown to be required for sustained growth of KRAS-mutant tumors and to constitute a diagnostic and prognostic biomarker of these tumors. Finally, we show how non-oncogene addiction of KRAS-mutant cancers to myeloid IL-1β can be abolished by pharmacologic inhibition of IL-1β or the VCAN target toll-like receptor (TLR) 2. Our findings can be directly translated to and tested in clinical trials of IL-1β inhibition against genomically stratified LUAD.

### Non-Oncogene Addiction of KRAS-Mutant Human and Murine Cancers to IL-1β

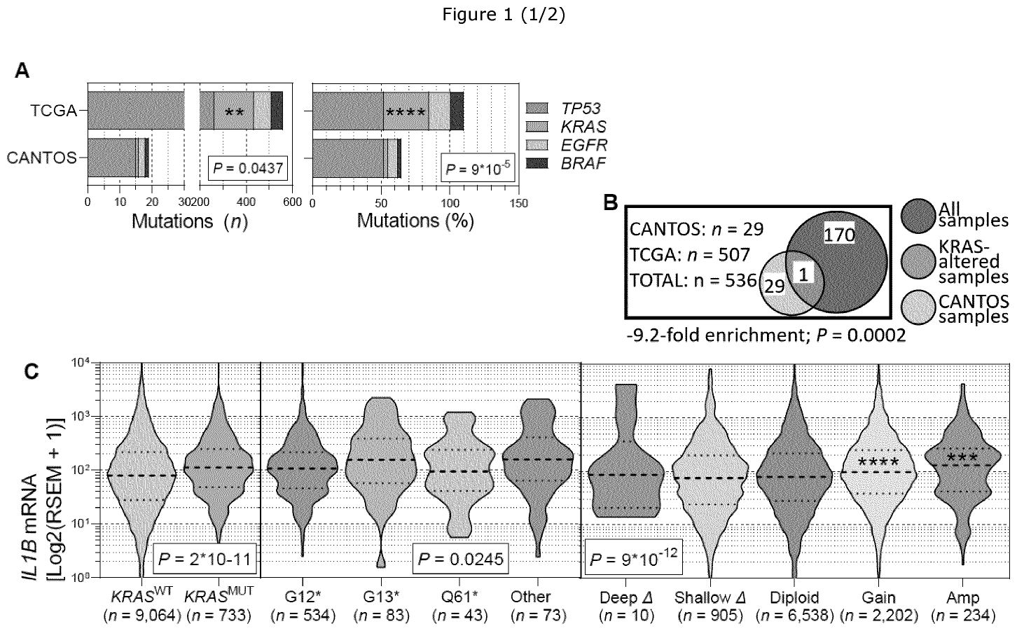

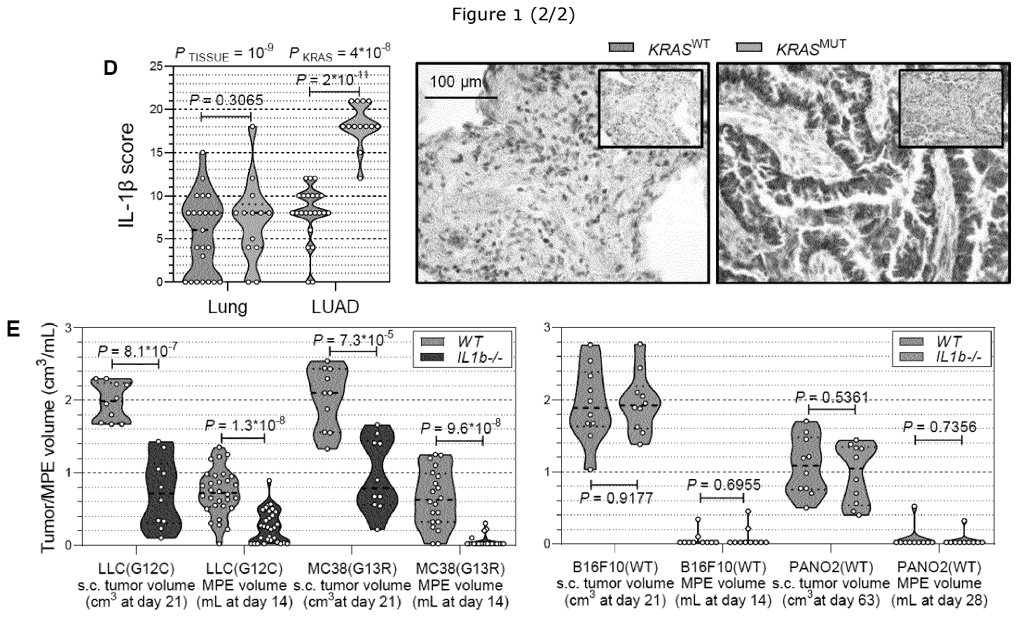

Puzzled by the negative results of the CANOPY-2 trial, we focused on published mutation data from incident LUAD from the CANTOS trial (11) and cross-examined them with the cancer genome atlas (TCGA) LUAD dataset (12), hypothesizing that IL-1β neutralization with canakinumab would specifically prevent the development of incipient KRAS-mutant (MUT) LUAD. Indeed, KRAS, but not TP53, EGFR, and BRAF, mutations were statistically significantly under-enriched in CANTOS versus TCGA patients (FIGS. 1A, B). We next analyzed TCGA pancancer transcriptome data to discover that IL1B mRNA levels were elevated in KRASMUT and amplified cancers, and performed IL-1β immunohistochemistry in our own patients with resected LUAD (13) to find increased IL-1β protein expression in KRASMUT LUAD compared with KRAS-wild-type (WT) LUAD and adjacent lung tissues (FIGS. 1C, D). We next injected C57BL/6 mice competent (WT) and diploinsufficient for Il1b alleles (Il1b−/−) (14) with syngeneic cancer cell lines carrying KrasWT and KrasMUT alleles (9, 10). Both subcutaneous (s.c.) and pleural routes of tumor cell injection were employed, since we previously identified that malignant pleural effusions (MPE) in mice are exclusively elicited by KrasMUT tumor cells (9, 10). All cell lines were verified for Kras, Mycoplasma Spp., and identity status multiple times during these investigations (FIGS. S1A, B). These experiments showed that specifically KrasMUT tumors were dependent on host IL-1β (FIG. 1E). Taken together, these results show that IL-1β neutralization prevents the development of incipient KRASMUT LUAD in humans, that KRASMUT human cancers contain elevated IL-1β levels, and that mouse KrasMUT cancers are specifically dependent on host IL-1β signaling, supporting the hypothesis of a selective non-oncogene addiction of KRASMUT cancers to IL-1β.

### Tumor-Associated Macrophages as a Source of Tumorigenic IL-1β

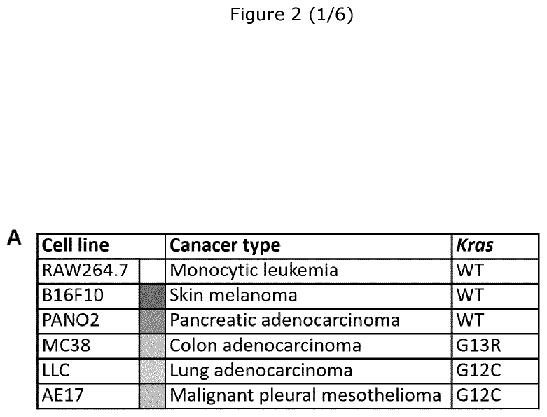

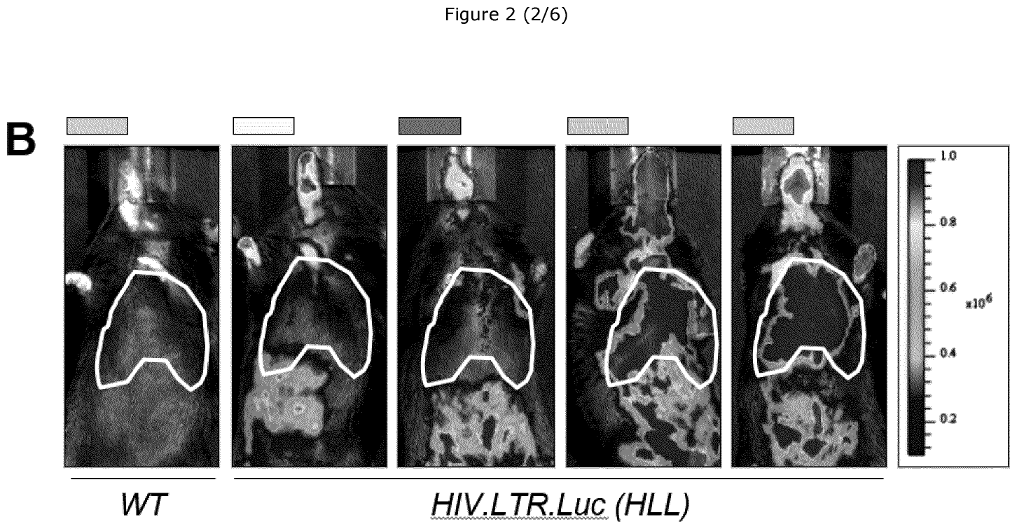

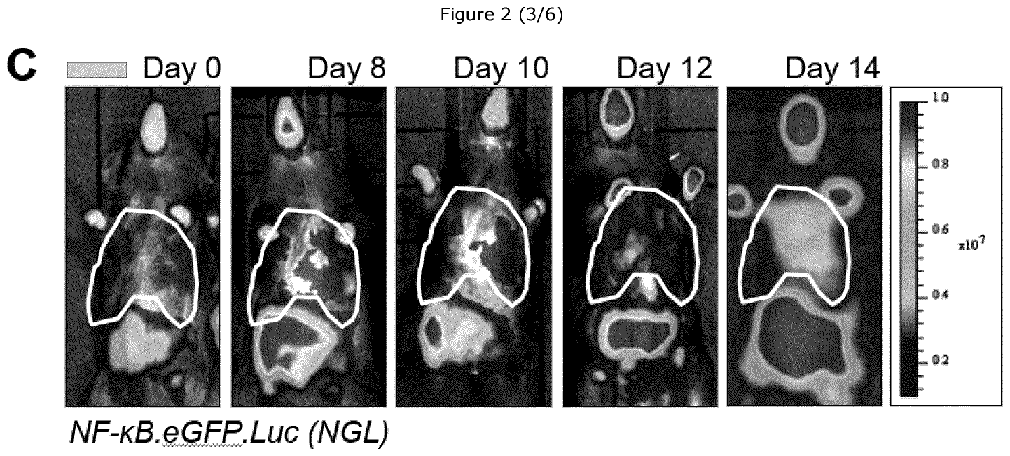

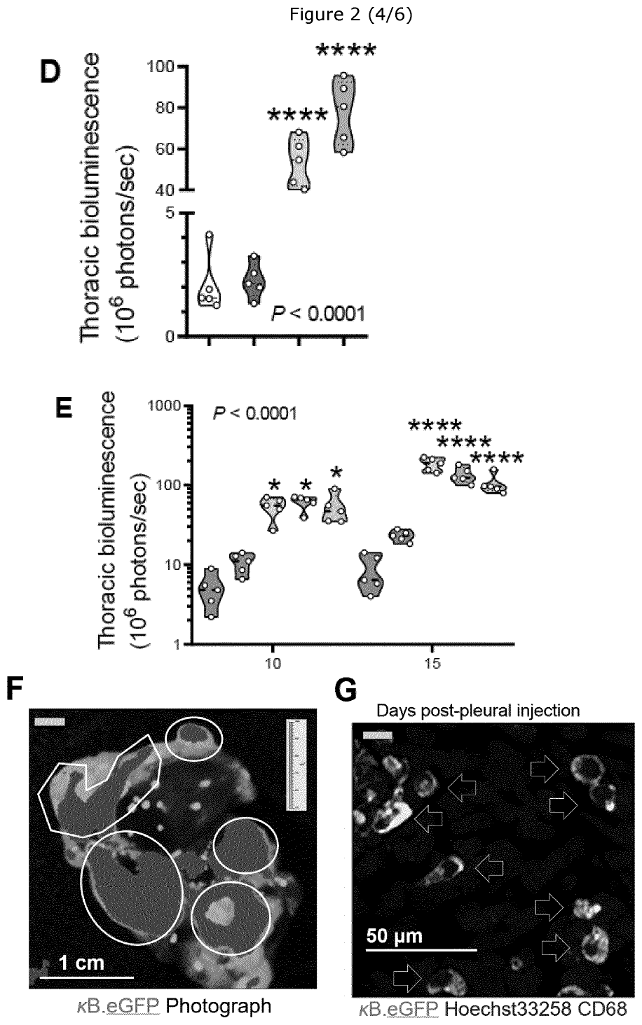

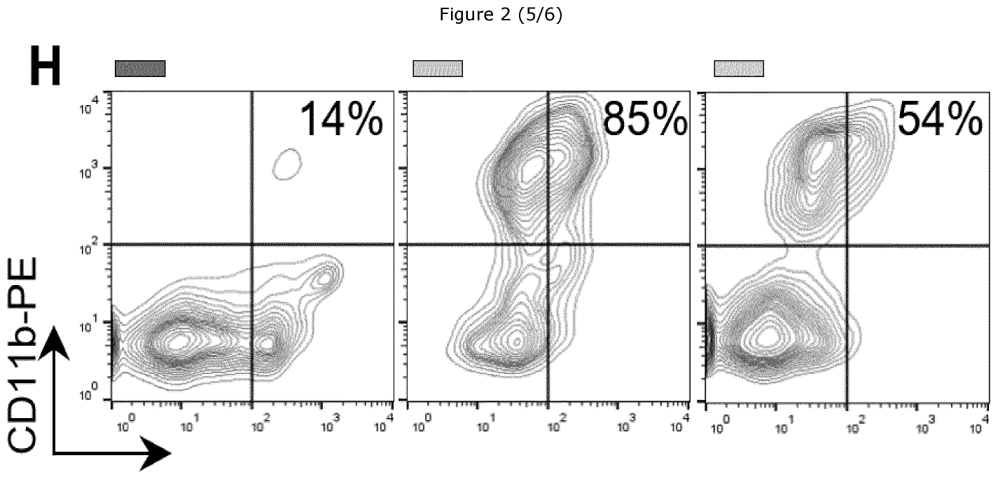

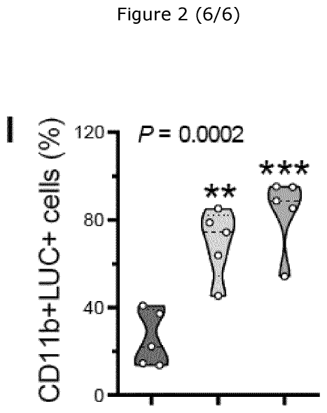

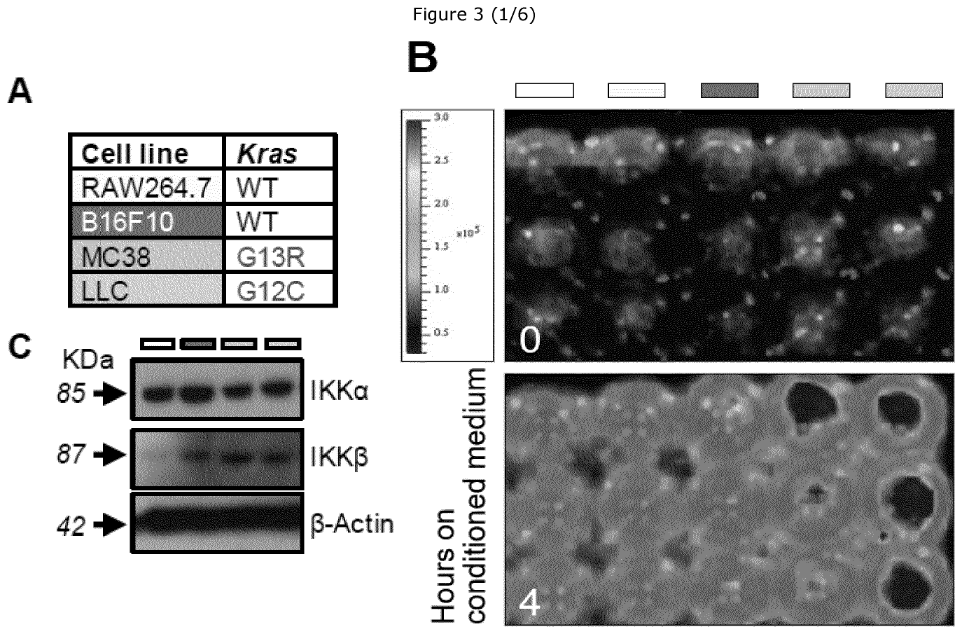

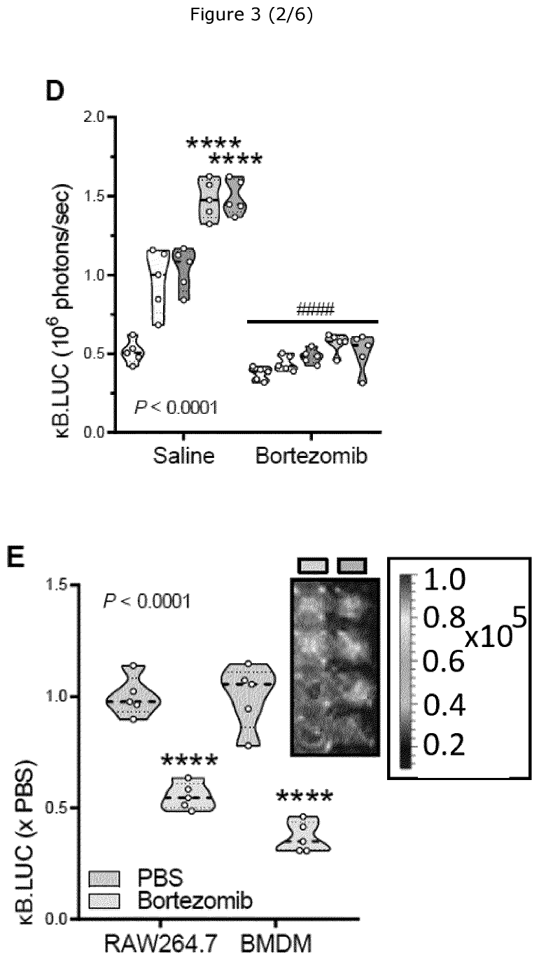

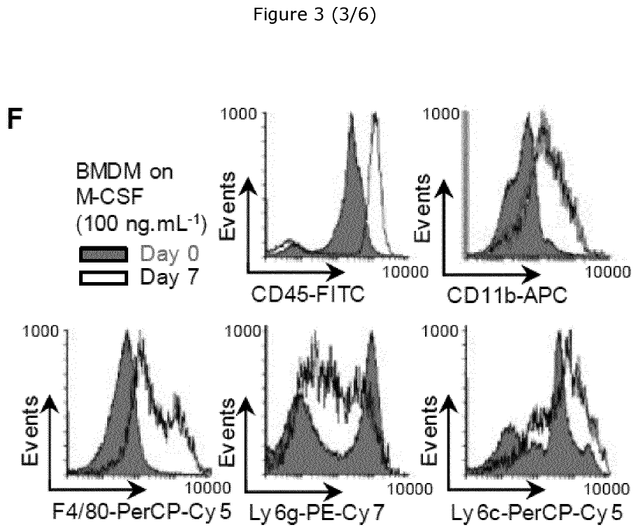

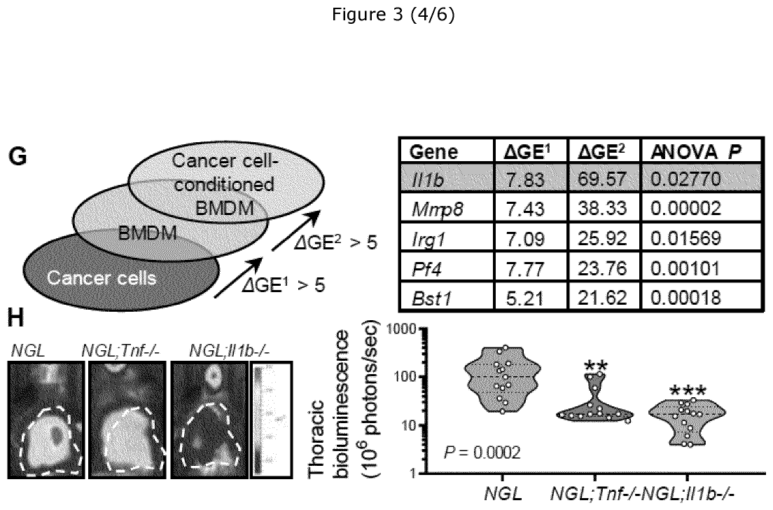

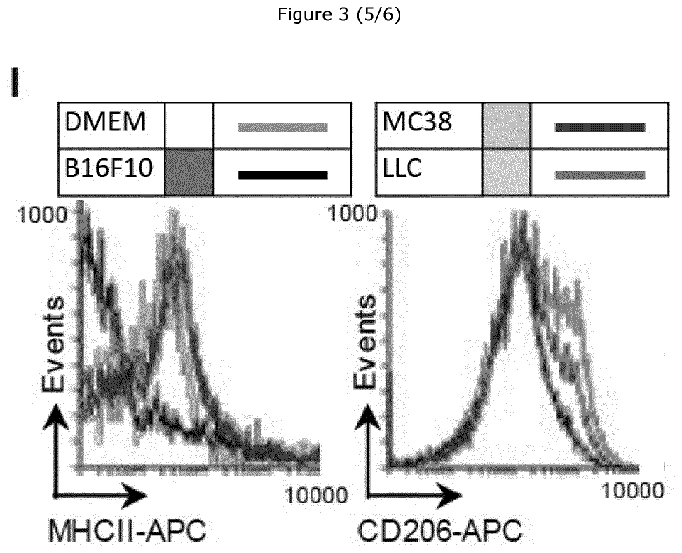

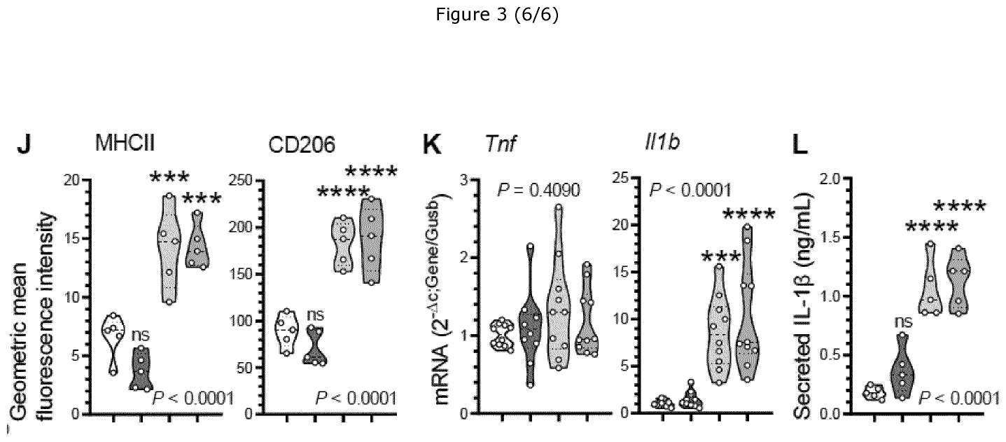

We next investigated the source of increased IL-1β in KRASMUT cancers, since both host immune and tumor cells are capable of IL-1β production (15-17). We were also based on previous work documenting that the IL-1β promoter lies under transcriptional control of NF-κB (18), a fact we validated in ChIPseq datasets from the ChIP-X Enrichment Analysis (CHEA) dataset (https://maayanlab.cloud/Harmonizome/dataset/CHEA+Transcription+Factor+Targets) and the ENCyclopedia Of DNA Elements (ENCODE) portal (https://www.encodeproject.org/) (FIG. S1C). For this, we first searched TCGA pan-cancer transcriptomes (n=10,071) for associations between mRNA levels of IL1B and established cancer and immune cellular lineage markers. IL1B mRNA levels were not correlated with mRNA levels of the neutrophil marker ELANE, the mast cell marker KIT, the fibroblast marker ACTA2, and the endothelial marker F8, were significantly associated with mRNA levels of KRAS per se, of the pan-lymphocyte marker CD3D, and the cancer cell marker KRT18, but showed the tightest correlation (coefficient=0.4; P<10-300) with mRNA levels of the macrophage marker ADGRE1 (FIGS. S2, S3). To further test this, we sought to identify the host cells that respond to KRASMUT tumor cells with NF-κB activation, since the transcription factor controls IL-1β transcription (18) and is central to innate immune responses (19). For this, we initiated in vivo screens of murine tumor cell lines with known Kras mutation status [(9), FIGS. 2A, S1] by transplanting them into two strains of bioluminescent NF-κB reporter mice expressing ubiquitous HIV-LTR. Luciferase (HLL mice) (20) or NF-κB.GFP.Luciferase (NGL mice) (21) transgenes. Pleural injections were selected for tumor cell inoculation because they generate MPE with overt cancer-induced inflammation (9, 10, 15). Serial imaging showed time-dependent NF-κB activation in host cells of recipient mice, conditional on the presence of Kras mutations in tumor cells (FIGS. 2B-E, S4A, B). The NF-κB reporter signal was emitted from pleural tumors and fluid, both containing cancer and immune cells (FIGS. 2F, S4C-F) (9, 10, 15). Histologic and flow cytometric analysis and quantification localized the NF-κB reporter signal to tumor-infiltrating macrophages of mice with KrasMUT pleural tumors and effusions (FIGS. 2G-I, S5-S7). Mast cells that foster MPE development (15) were not involved in the observed NF-κB response (FIG. S8A, B). Time-dependent NF-κB activation in host cells was stronger in pleural compared with s.c. tumor models, and required expression of mutant Kras by tumor cells (FIGS. S8C, D and S9A-C). Adoptive bone marrow transfer corroborated myeloid cells as the origin of tumor-induced NF-κB activation, and pharmacologic killing of pleural macrophages prevented host NF-κB activation and pleural carcinomatosis (FIGS. S10A-E and S11A, B). The pro-tumor function of pleural macrophages was also consistent with the phenotype of macrophage-depleted Lyz2.Cre; Dta mice (22) (FIGS. S11C, D). Tumor-secreted solute factors are responsible for NF-κB activation in macrophages, since murine RAW264.7 macrophages stably expressing the NGL reporter responded with robust in vitro NF-κB activation to cell-free media conditioned by KrasMUT, but not by KrasWT or Krassilenced, tumor cells (FIGS. 3A, B and S9A, D, E). This NF-κB response requires canonical NF-κB signaling, since it involved IKKβ and was attenuated by the proteasome inhibitor bortezomib (FIGS. 3C, D, S9A, D, E and S12A-C). Proteasome-dependent canonical NF-κB activity was also documented in bone marrow-derived macrophages (BMDM) derived from NGL mice (FIGS. 3E, F). Differential gene expression (ΔGE) analyses (GEO datasets GSE94847, GSE94880, GSE130624, and GSE130716; total n=32) identified 13 BMDM-specific transcripts that were further induced by incubation with tumor-conditioned media (ΔGE>5; ANOVA P<0.05) and included Il1b but not Il6 and Tnf reported elsewhere (23) (FIG. 3G and Table 1). In addition, NGL mice diploinsufficient in Il1b alleles (14) were resistant to tumor-induced NF-κB activation (FIG. 3H). Incubation of BMDM with KrasMUT tumor-conditioned media promoted their differentiation as assessed by flow cytometry for markers MHCII and CD206, and Il1b mRNA and IL-1 protein expression (FIGS. 2I-L). These data directly show that KRASMUT tumor cells can activate NF-κB in macrophages via solute mediator(s) that trigger IKKβ-mediated NF-κB activation, differentiation, and IL-1β elaboration.

### Tumor-Secreted Versican as a Key Macrophage Effector

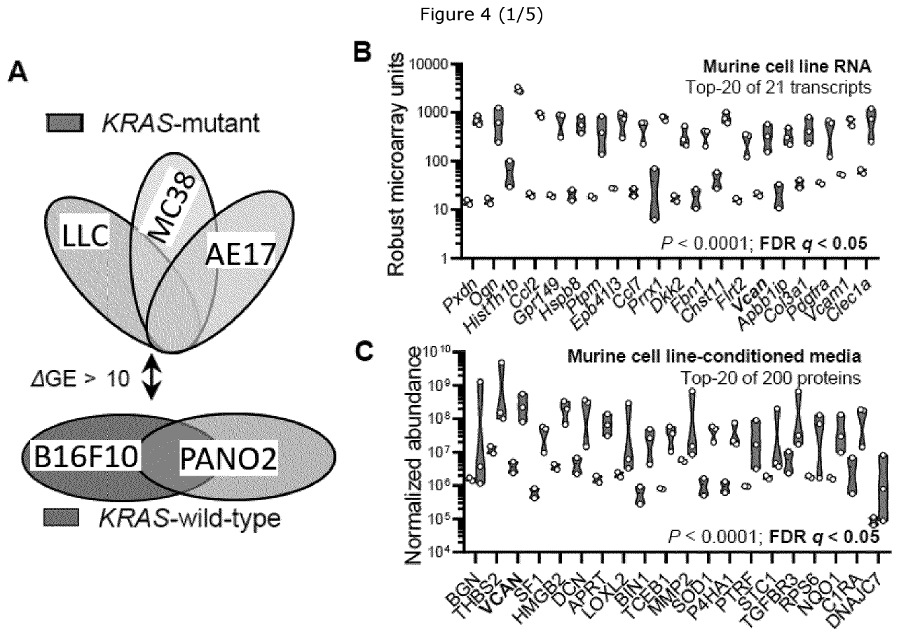

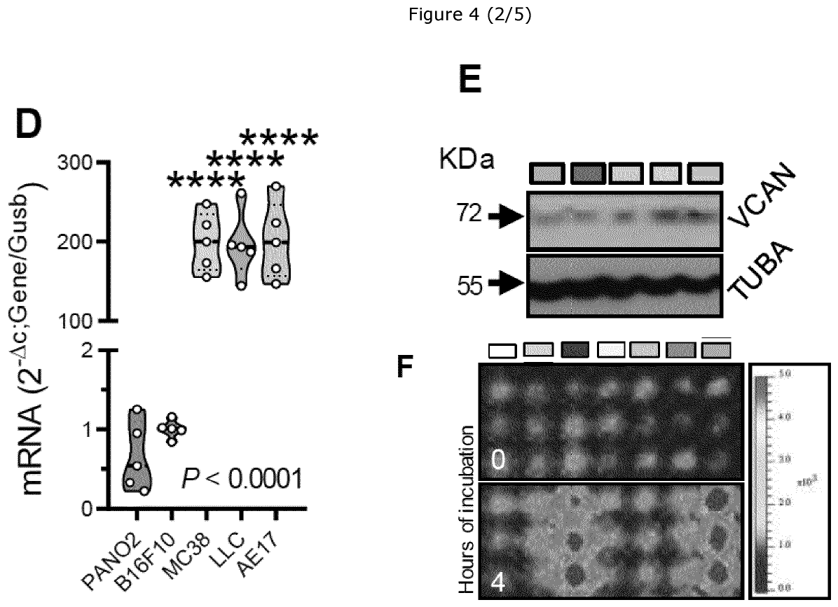

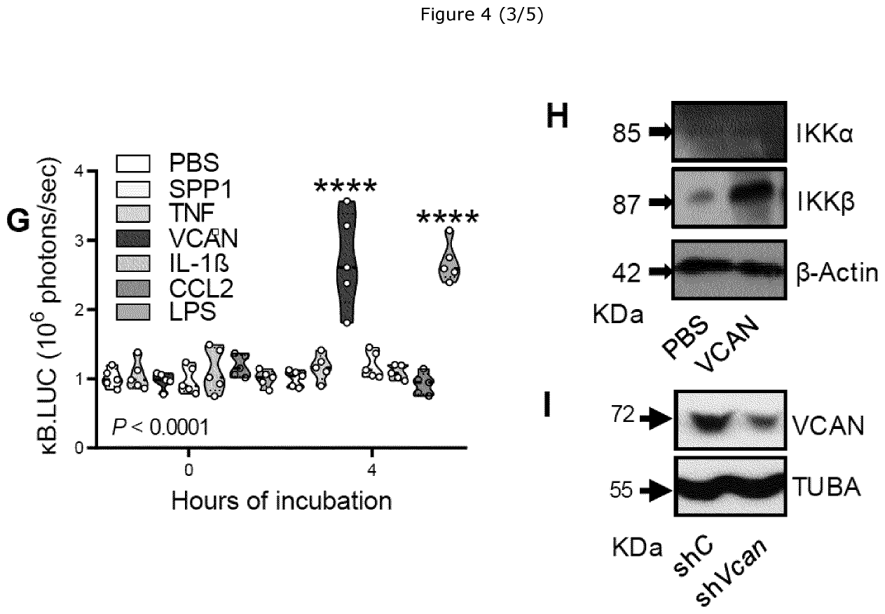

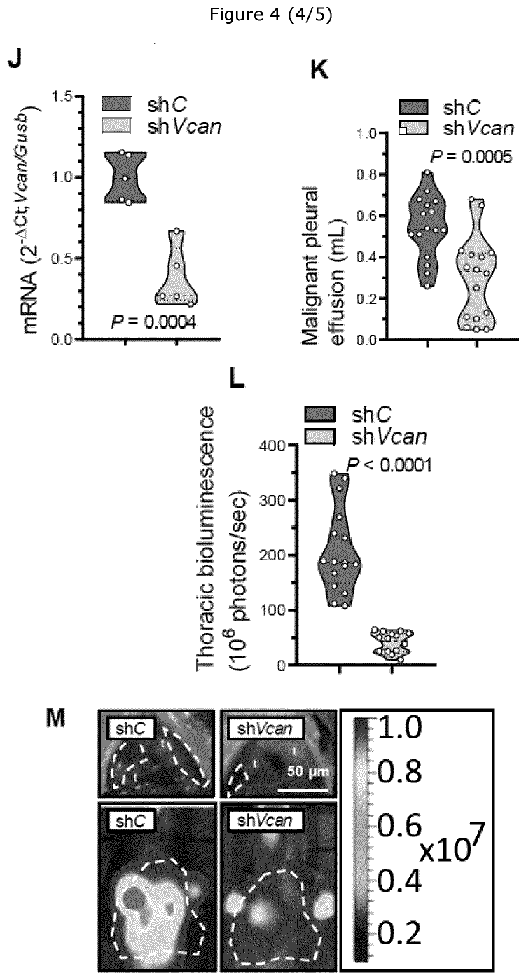

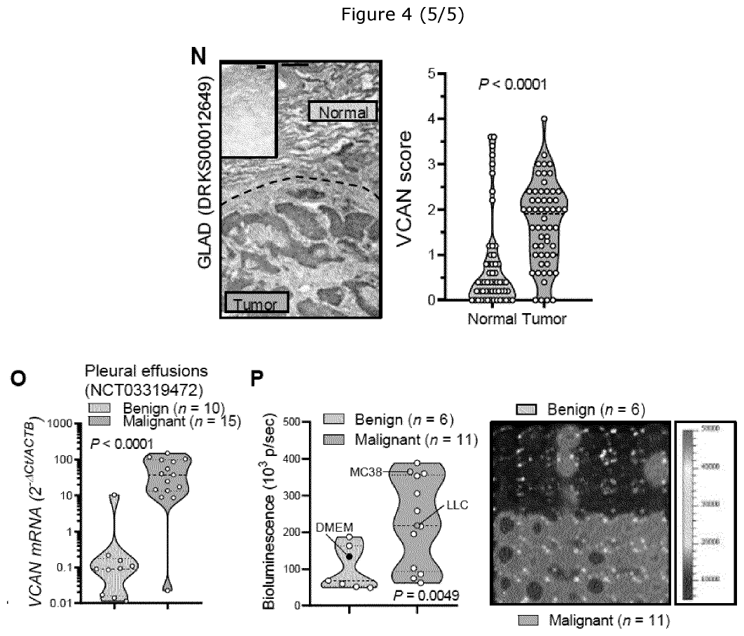

We next compared KrasMUT with KrasWT cancer cells for secretory molecules triggering macrophage NF-κB activation. Microarrays identified transcripts over-represented in Kras25 MUT tumor cells, and a proteomic screen of tumor cell-conditioned media detected 226 proteins secreted >10-fold by KrasMUT over KrasWT cells, with the glycoprotein versican (VCAN; encoded by the human/murine VCAN/Vcan genes) emerging from both screens and withstanding validation (FIGS. 4A-E, S12D-E, Table 2). Multiple NF-κB ligands were also screened using pNGL-expressing RAW264.7 macrophages, revealing that the toll-like receptor (TLR) 2 ligand VCAN potently activates macrophage NF-κB-driven transcription to the same degree as the TLR4 ligand lipopolysaccharide (LPS) (FIGS. 4F, G). VCAN also induced IKKβ in primary murine BMDM, which were verified by microarray to overexpress >10-fold over cancer cells TLR1, TLR2, TLR6-9, and TLR13 (FIGS. 4H, S12F-G, and S13A-C). Importantly, shRNA-mediated Vcan silencing in LLC cells diminished their ability to trigger NF-κB activation in NGL mice and to precipitate MPE (FIGS. 4I-M and S12I-J). VCAN overexpression is not restricted to mouse KrasMUT cancers, since VCAN transcripts are also overrepresented in human cancers with high KRASMUT frequencies (derived from the catalogue of somatic mutations in cancer, COSMIC), such as LUAD from smokers (GEO dataset GSE43458), and NSCLC and colorectal adenocarcinoma (COAD/READ; GEO dataset GSE103512) (FIGS. S14A-B) (24-26). High VCAN mRNA expression also portended poor survival in a number of human cancers from the KMplot pan-cancer RNAseq dataset (http://kmplot.com/; FIGS. S14C and S15A-F) (27). Analysis of samples from two of our own clinical studies (13, 28) showed that VCAN protein expression was significantly increased in LUAD compared with adjacent lung tissues and that VCAN mRNA expression was significantly increased in human MPE compared with benign pleural effusions (BPE) (FIGS. 4N and O). To test whether the proposed inflammatory loop can serve as a diagnostic tool to distinguish MPE from BPE, which is an unmet clinical need (28), pNGL-expressingRAW264.7 macrophages were exposed to cell-free supernatants from human pleural effusions. After 4 hours, a robust NF-κB reporter signal was triggered selectively by MPE supernatants (FIG. 4P). Taken together, these data indicate that VCAN secreted by cancer cells triggers IKKβ-mediated NF-κB activation in tumor-associated macrophages and promotes metastasis. Moreover, VCAN is overexpressed in human KRASMUT cancers and can serve as a diagnostic and prognosis biomarker.

### Myeloid IKKβ as the VCAN Accessory

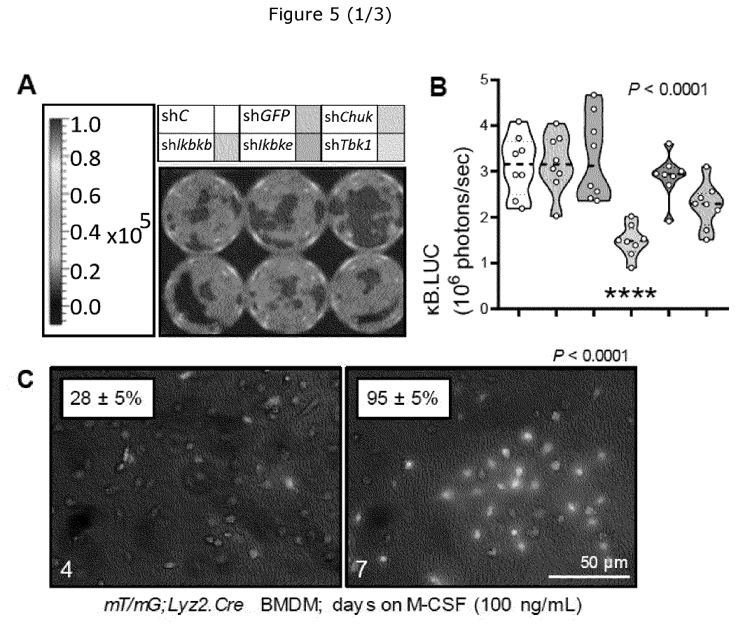

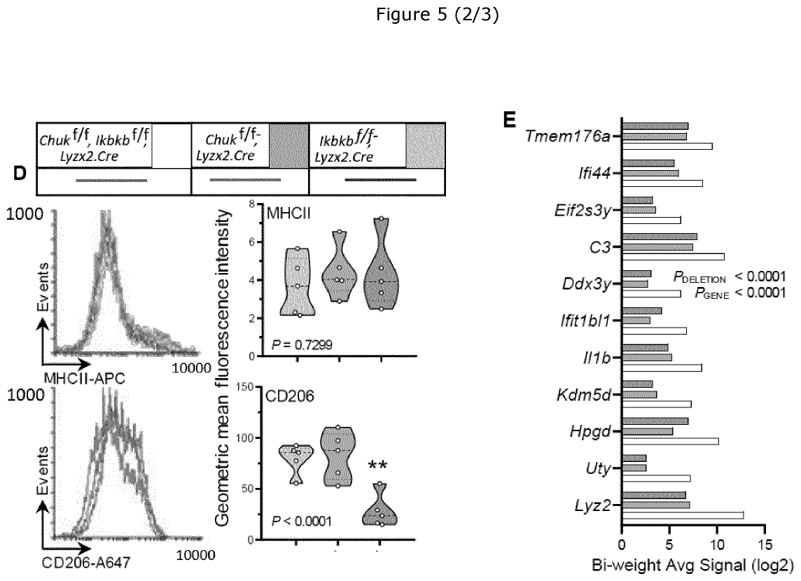

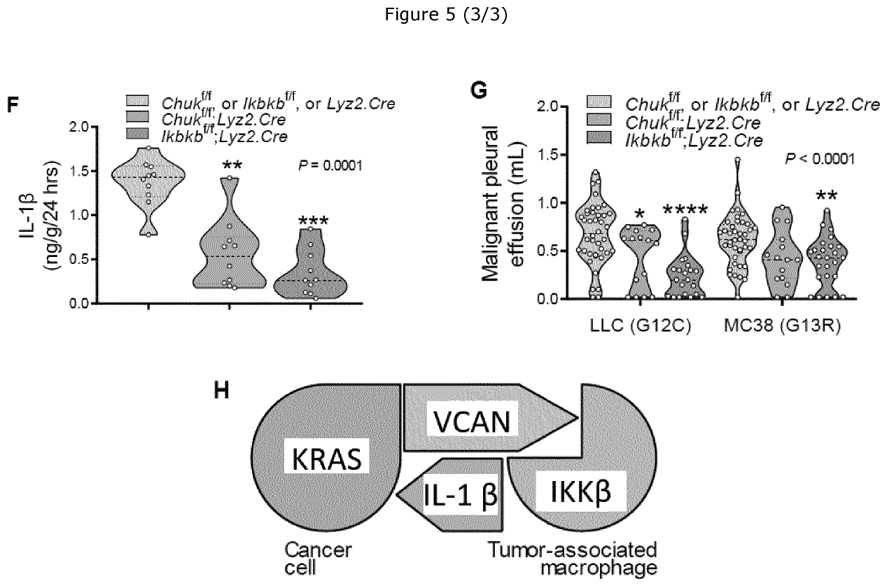

To identify the IKK responsible for NF-κB signaling in macrophages, we silenced the four main IKKs (encoded by the murine Chuk, Ikbkb, Ikbke, and Tbk1 genes) in RAW264.7 macrophages and identify IKKβ as the main mediator of NF-κB activation in these cells (FIGS. 5A-B). To further define myeloid IKKβ functions, we obtained BMDM from intercrosses of Lyz2.Cre mice with mice carrying conditionally-deleted alleles of IKKα (Chukf/f) and IKKβ (Ikbkbf/f), as well as with Cre-reporter mice switching from red to green fluorescence upon Cre-mediated recombination (mT/mG), all reported previously (22, 29). Treatment of bone marrow cells from mT/mG; Lyz2. Cre mice with macrophage-colony stimulating factor (M-CSF; 100 ng/ml) to drive them towards macrophage differentiation and lysozyme 2 (LYZ2) expression yielded efficient Cre-mediated recombination (FIG. 5C). Flow cytometric assessment of BMDM derived from these mice showed that intact IKKβ signaling in primary macrophages is essential for their differentiation and expression of critical proinflammatory genes including Lyz2, Il1b, and C3 (FIGS. 5D-F and Table 3). Finally, two different syngeneic KrasMUT tumor cell lines featuring VCAN overexpression were inoculated into the pleural space of the above myeloid IKK-deleted mice, to reveal that intact IKKβ signaling in macrophages is required for MPE (FIG. 5G). Thus, VCAN-driven IKKβ activation mediates NF-κB signaling, IL-1β expression, differentiation, and pro-tumor function of macrophages (FIG. 5H). To further query the proposed KRAS-VCAN-IKKβ connection, we interrogated mutations, copy number alterations, and fusions of the encoding genes in the TCGA pan-cancer dataset. Interestingly, VCAN and IKBKB alterations (mostly missense mutations) each occur in 5% of all cancer patients, and are significantly mutually enriched (VCAN in KRAS and IKBKB in VCAN mutations) suggesting mutual addiction (FIGS. S16A-C). In addition, KRAS, IKBKB, and VCAN alteration frequencies across 32 human cancer types are tightly correlated, and were highest in LUAD, COAD/READ, and uterine corpus endometrial carcinoma (UCEC), cancers that commonly cause MPE (FIG. S16D). In the latter tumor types featuring KRAS, IKBKB, and VCAN alteration frequencies, addiction of IKBKB and VCAN mutations persisted, and patients with VCAN and/or IKBKB-altered cancers displayed decreased body mass (cachexia), higher mutation burden, microsatellite instability, and hypoxia indices (FIGS. S17A-C). Collectively, these data support that tumor cell VCAN cooperates with myeloid IKKβ in mouse and human cancers.

### Non-Oncogene Addiction of KRAS-Mutant Tumors to IL-1β is Actionable

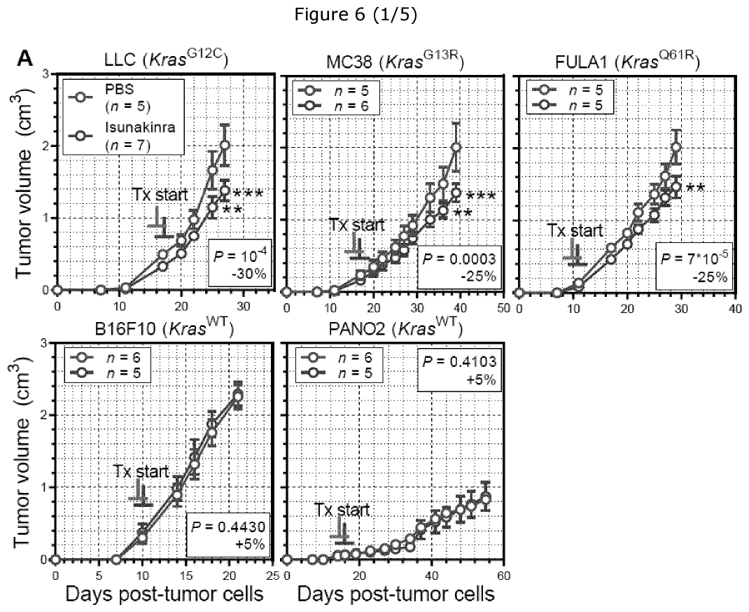

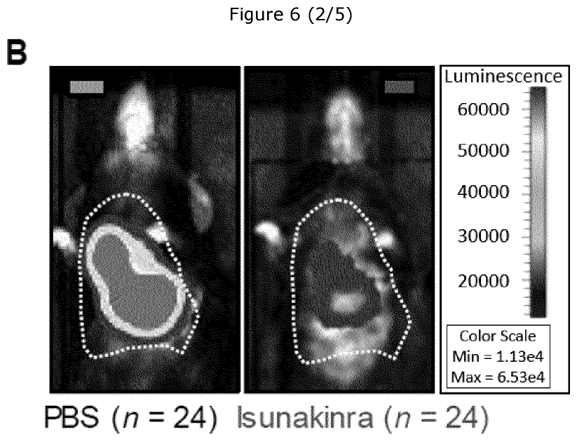

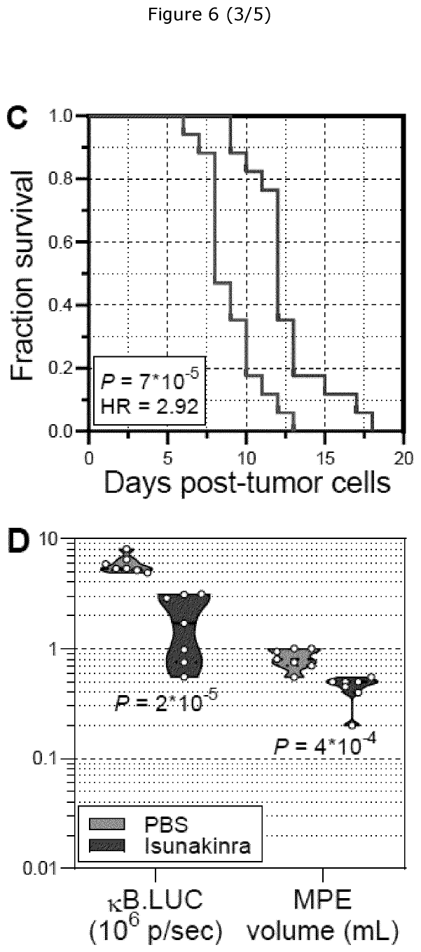

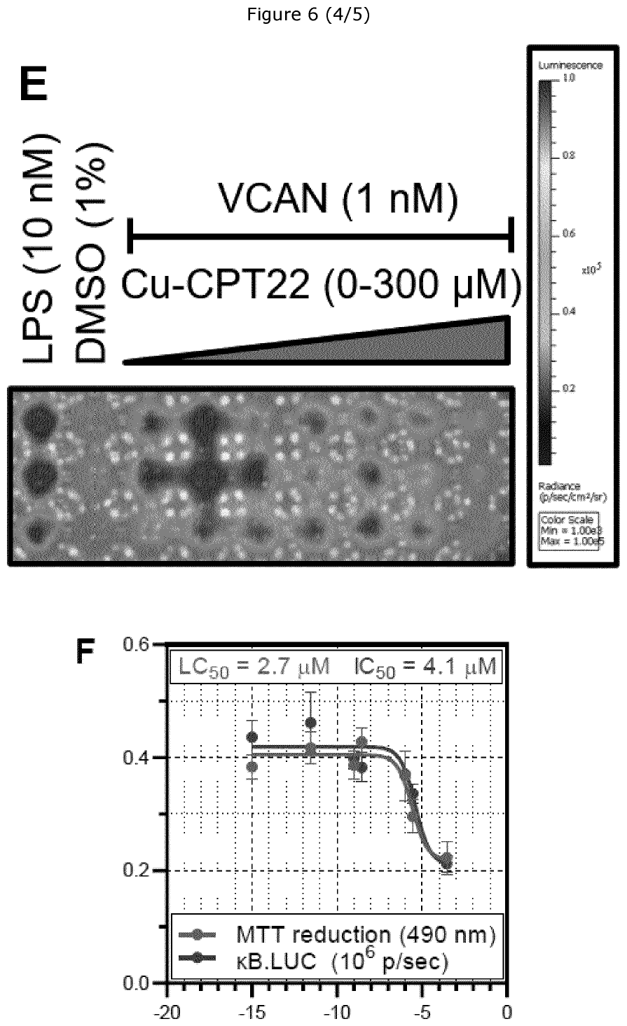

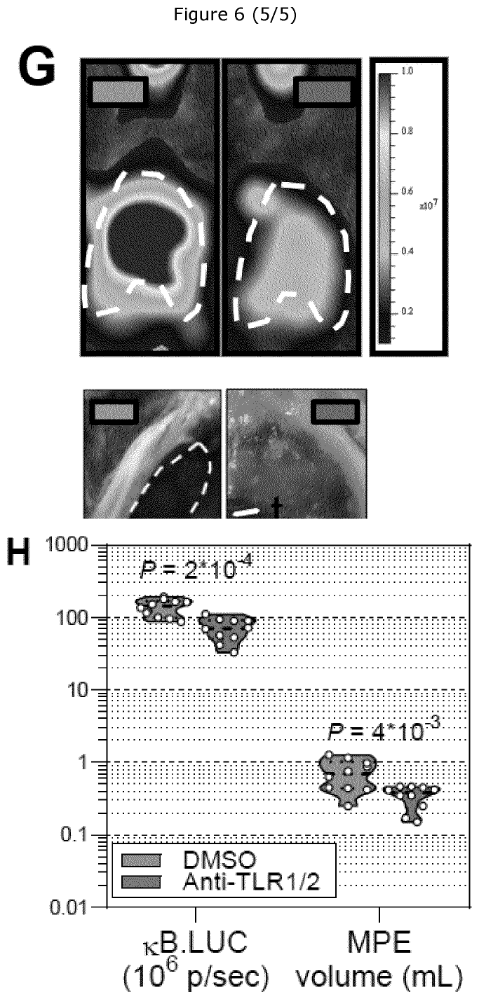

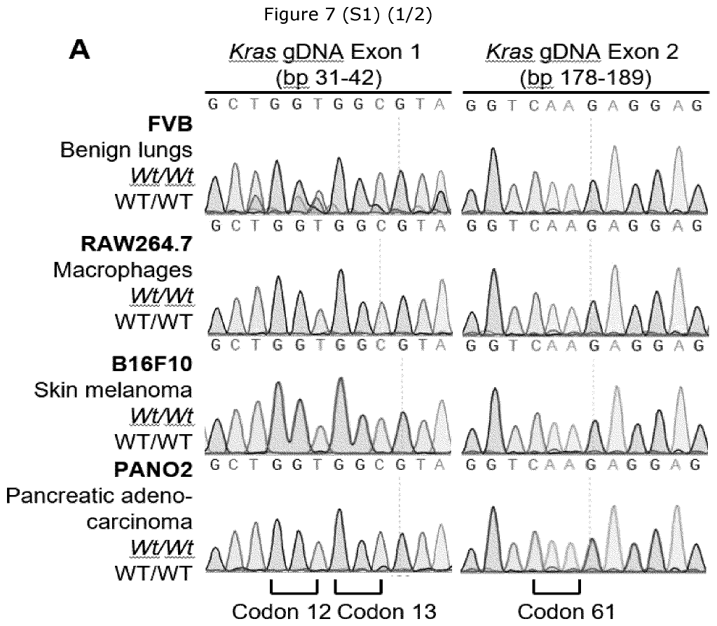

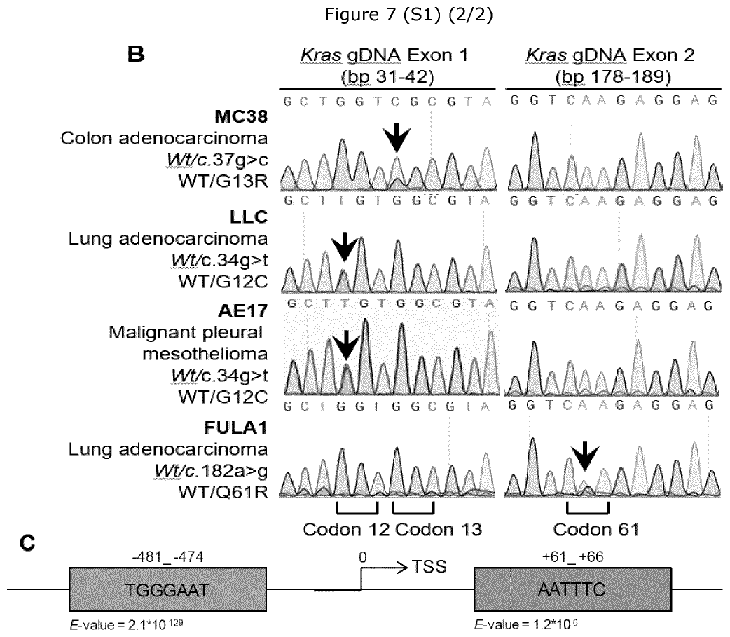

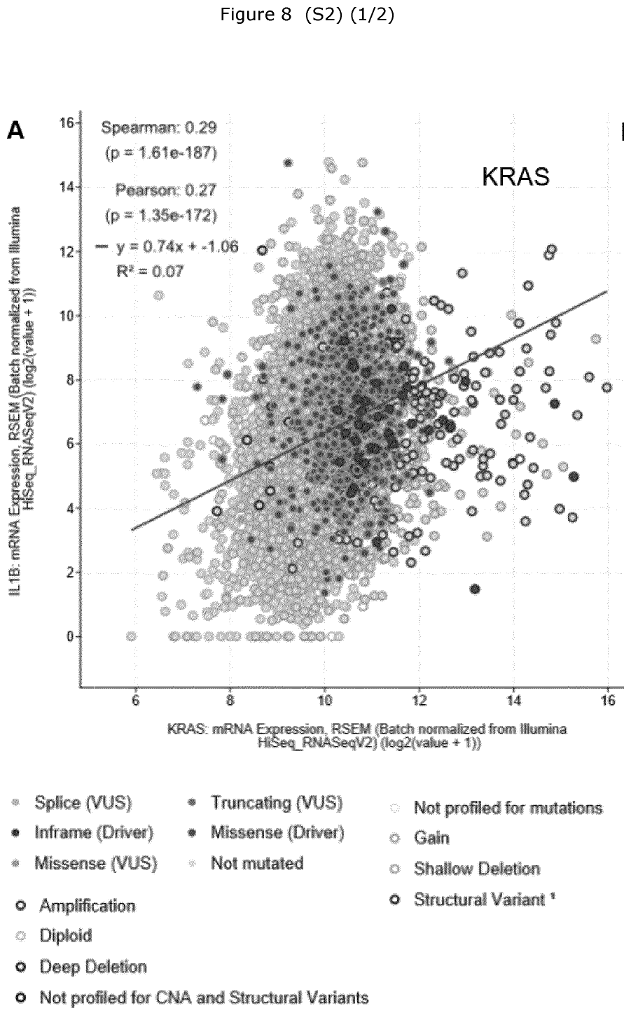

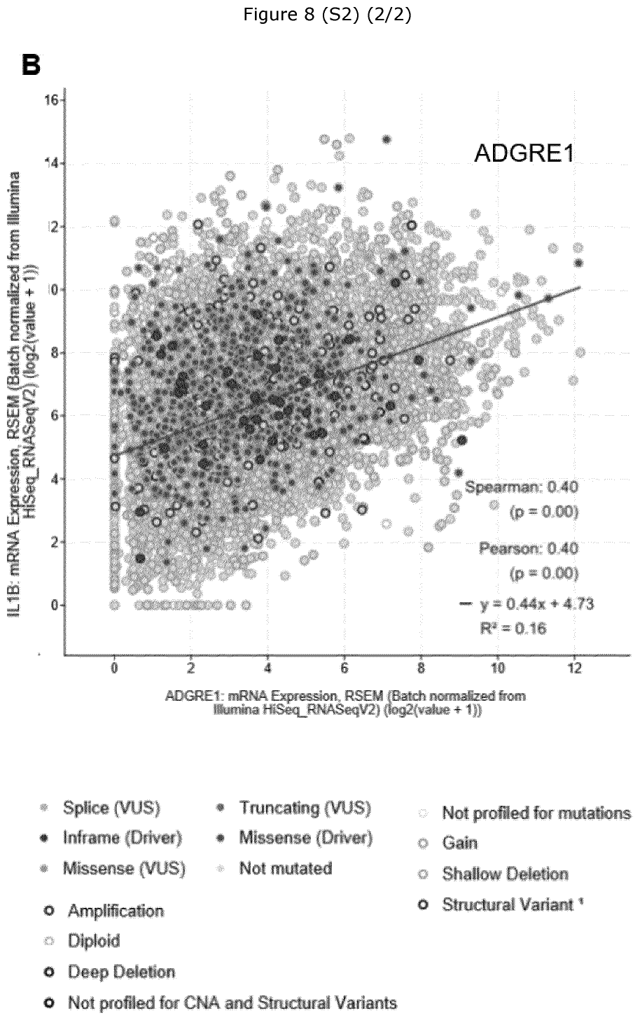

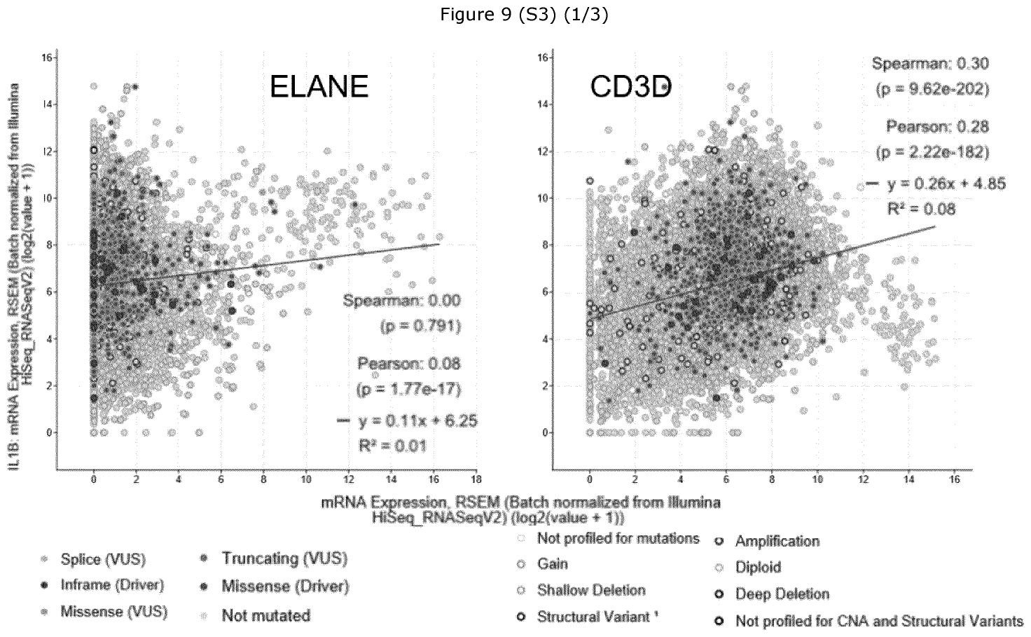

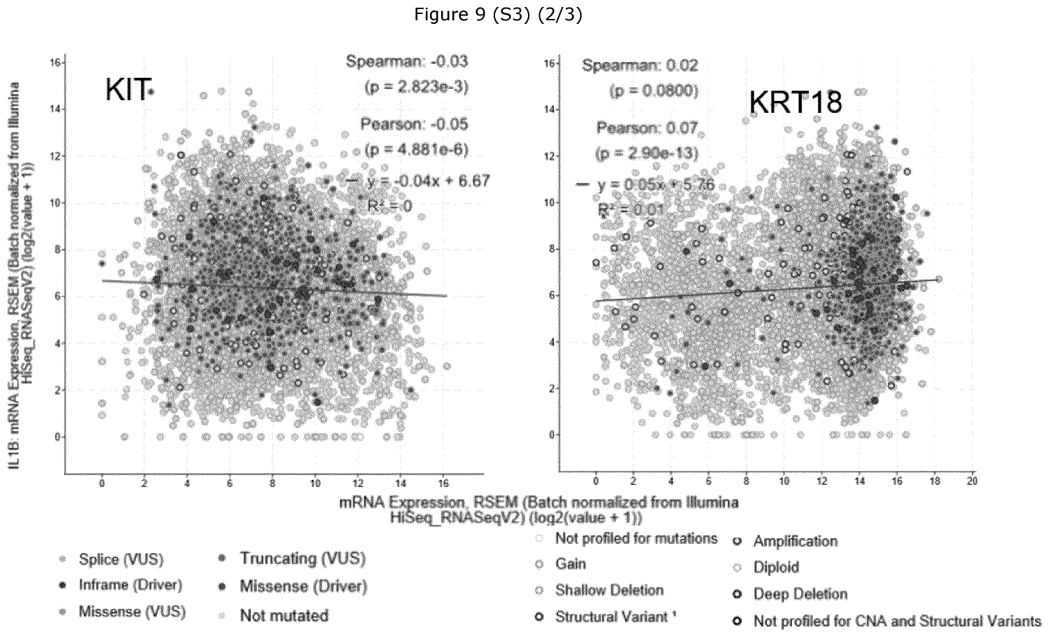

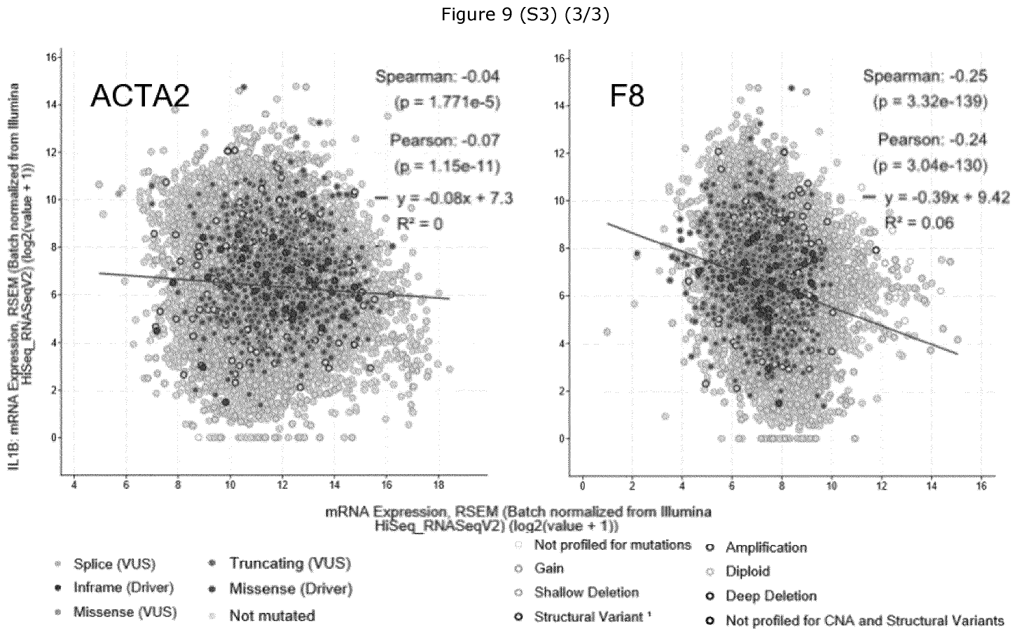

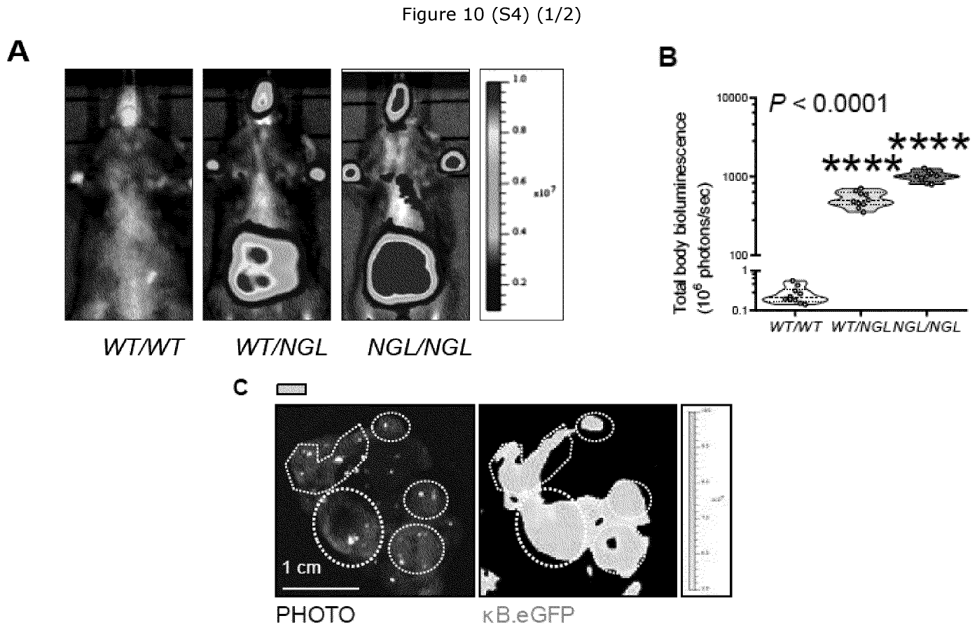

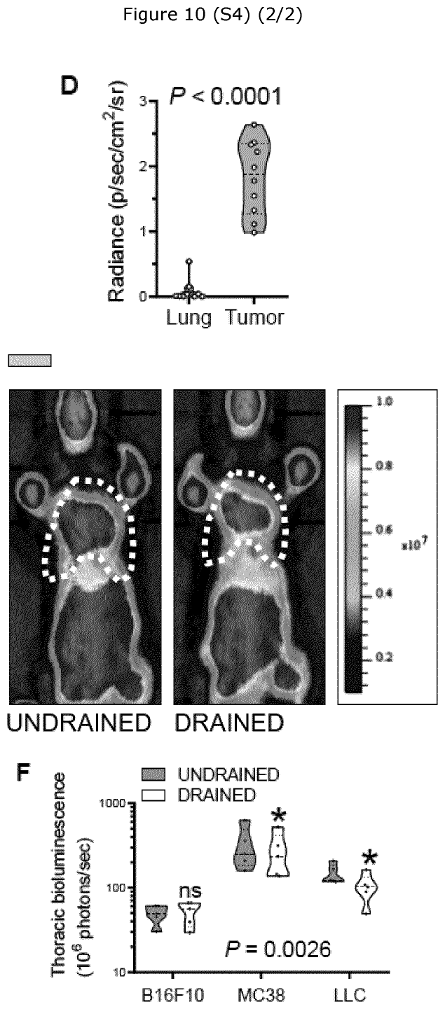

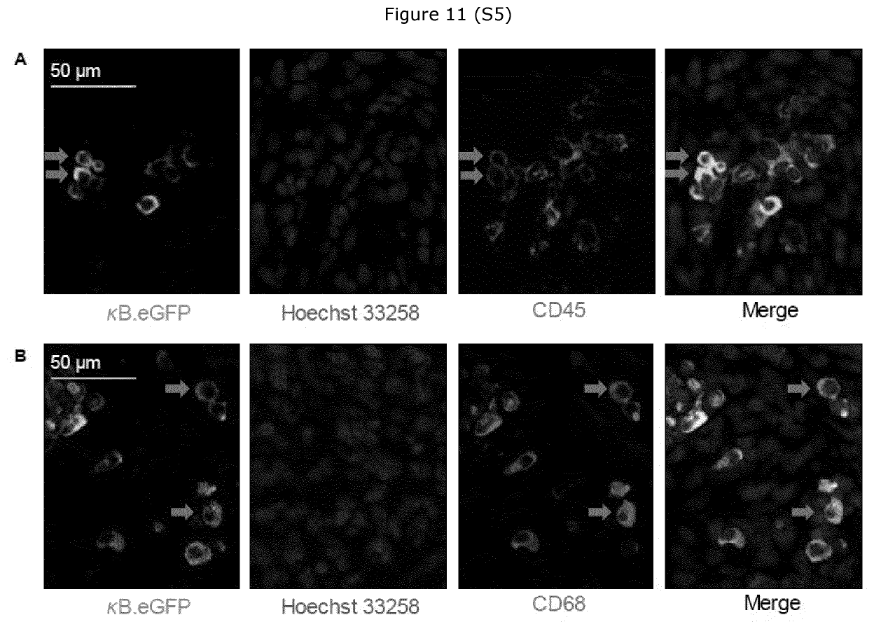

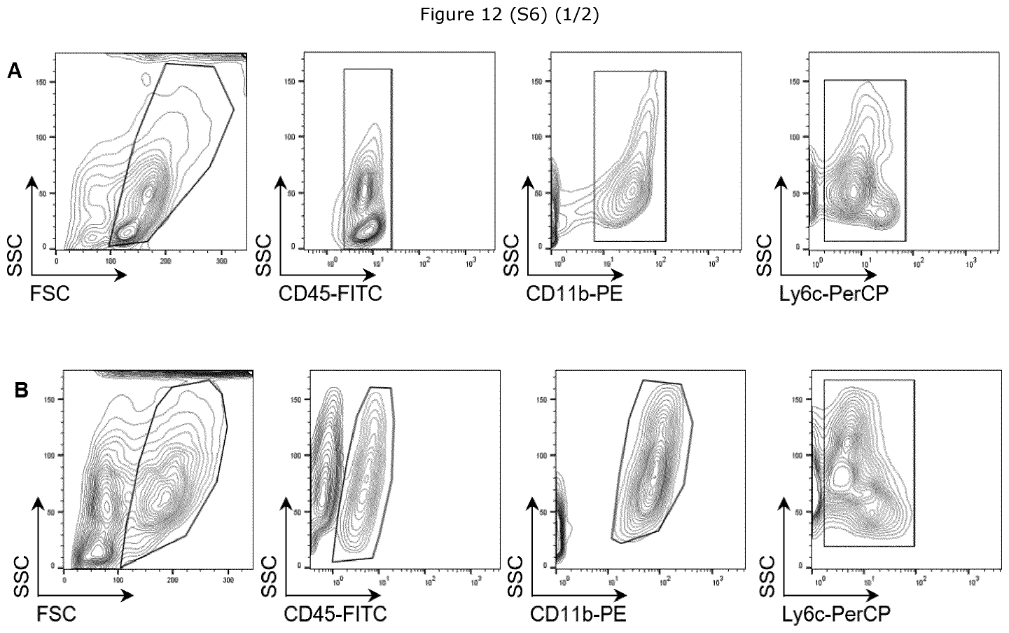

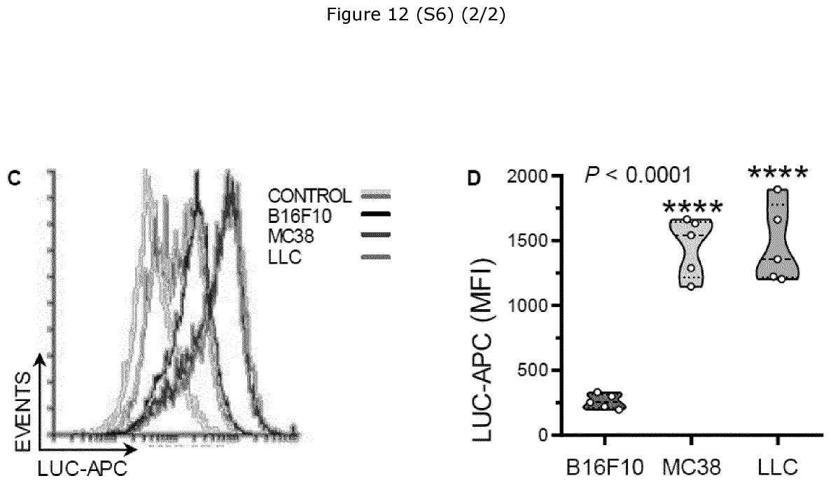

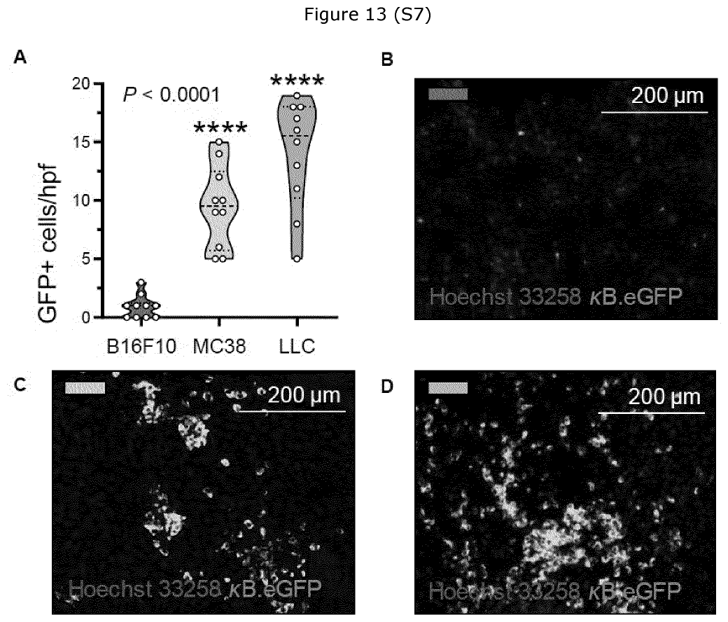

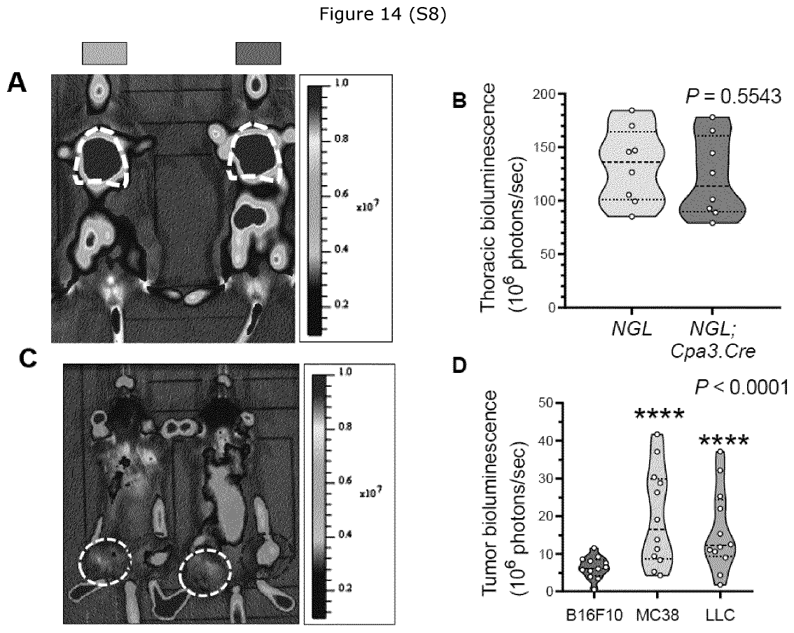

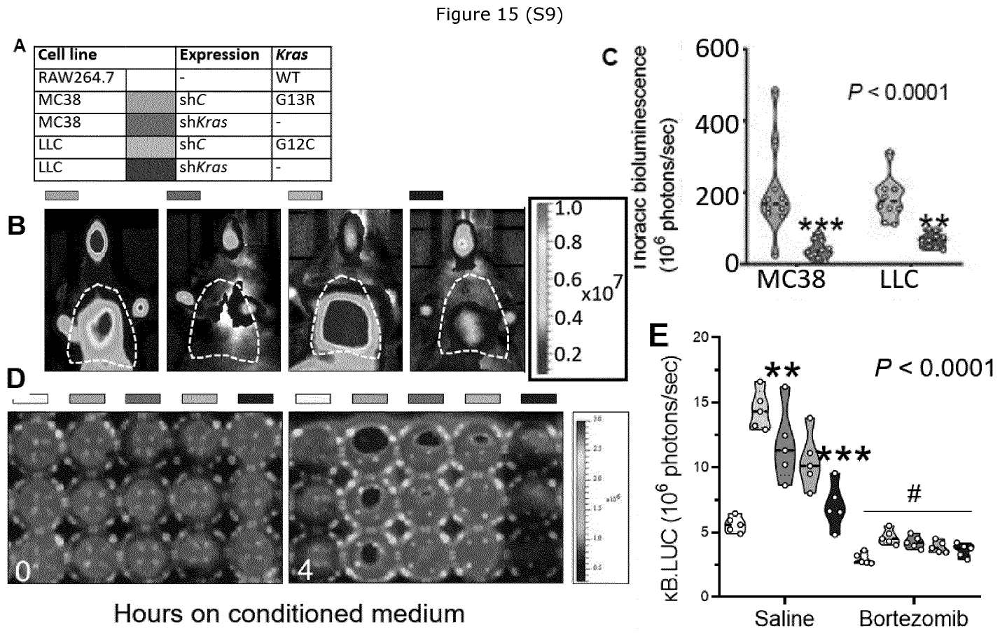

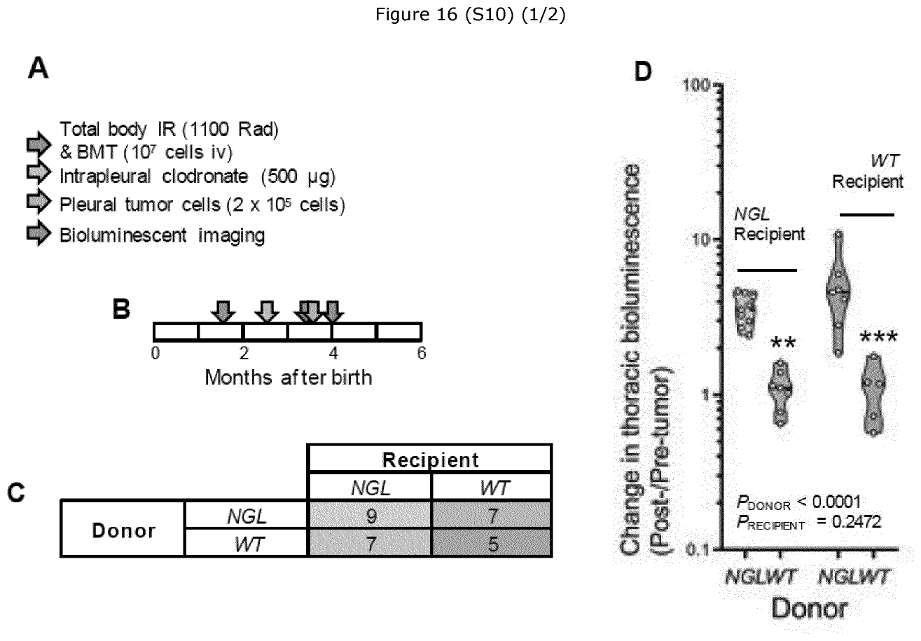

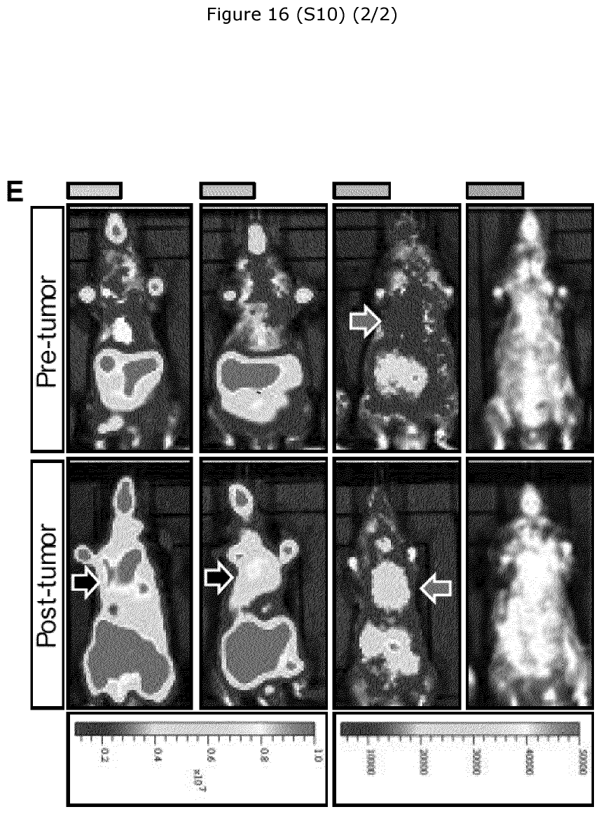

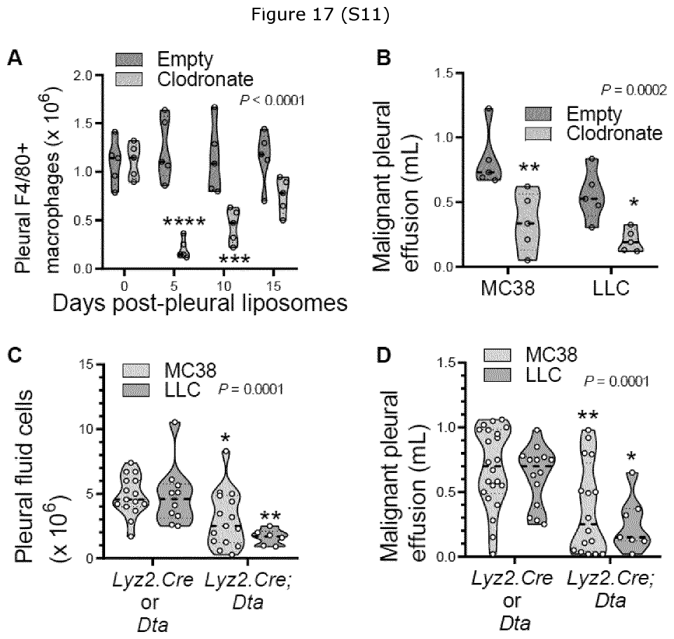

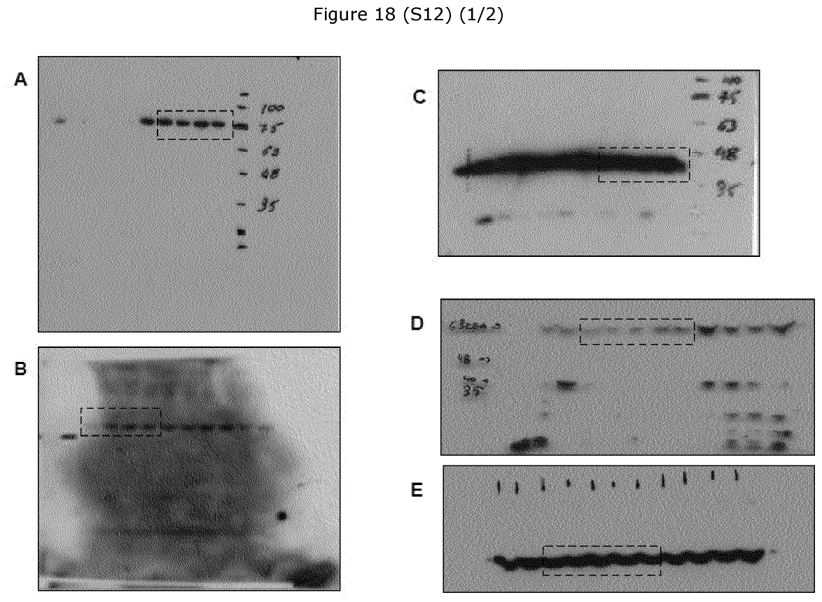

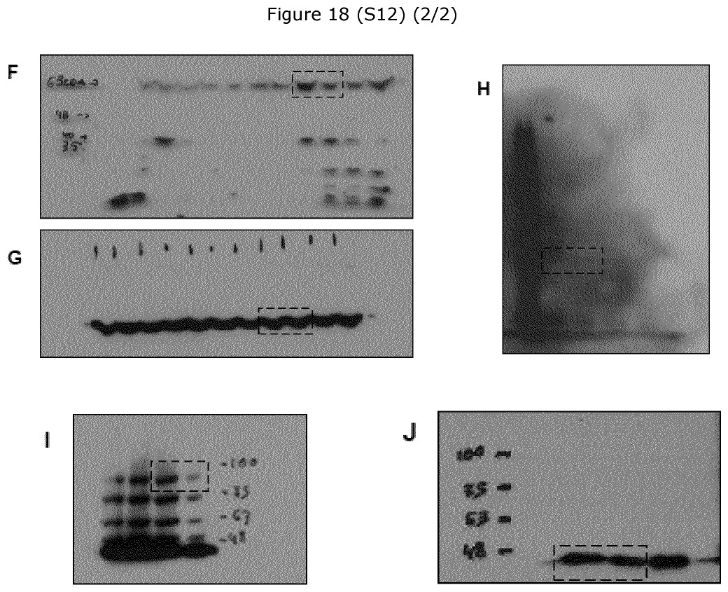

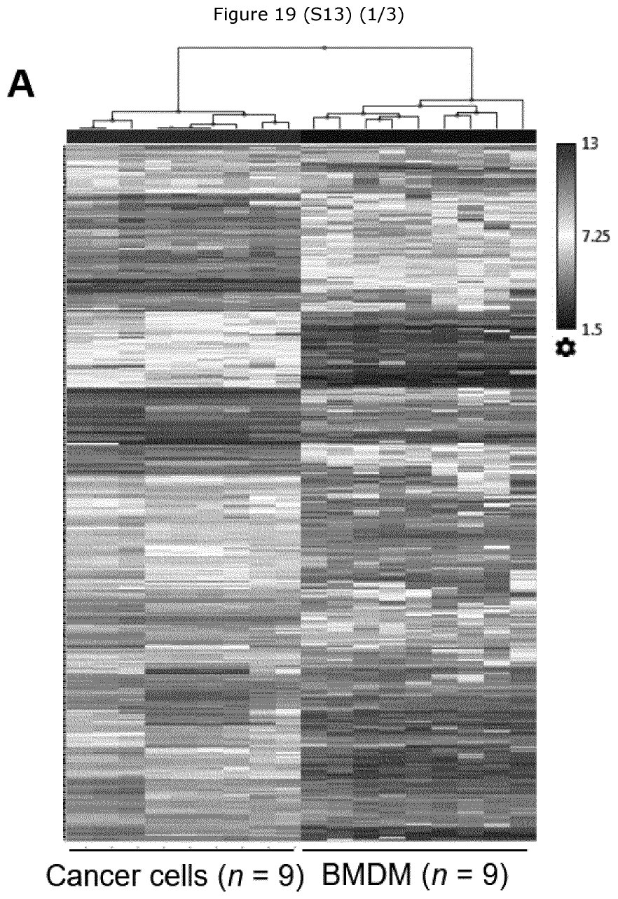

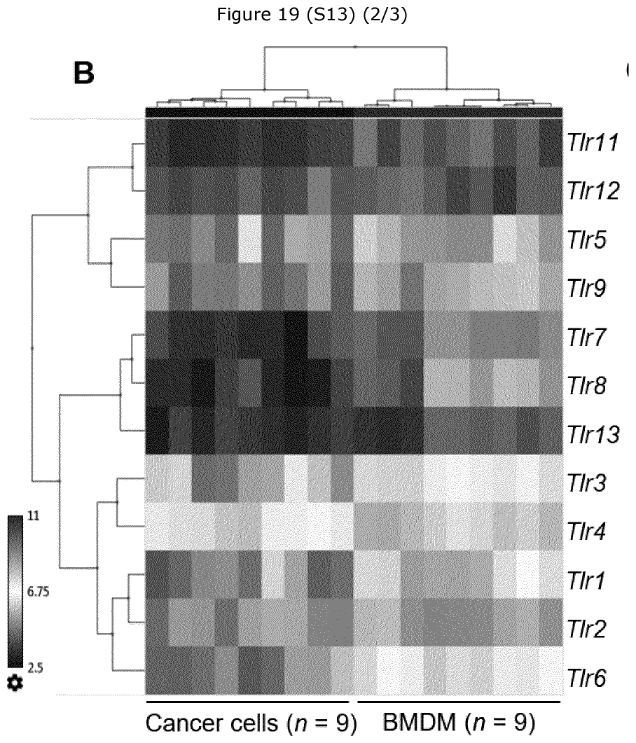

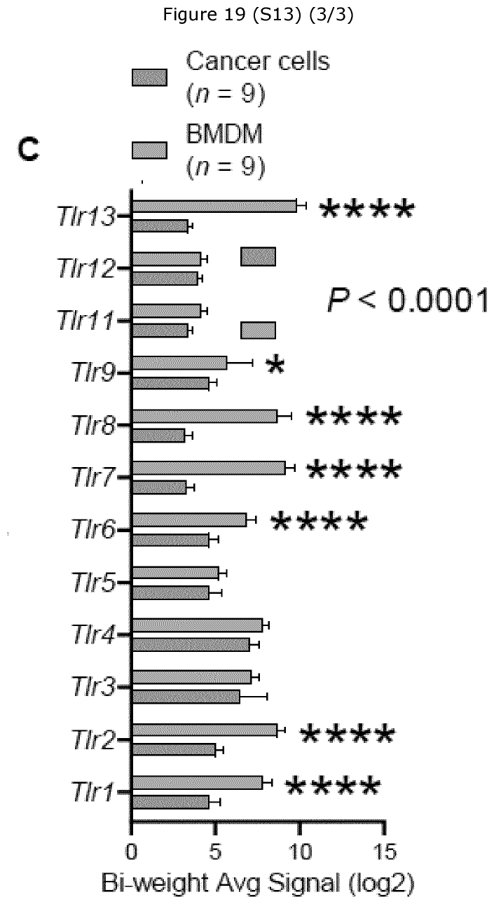

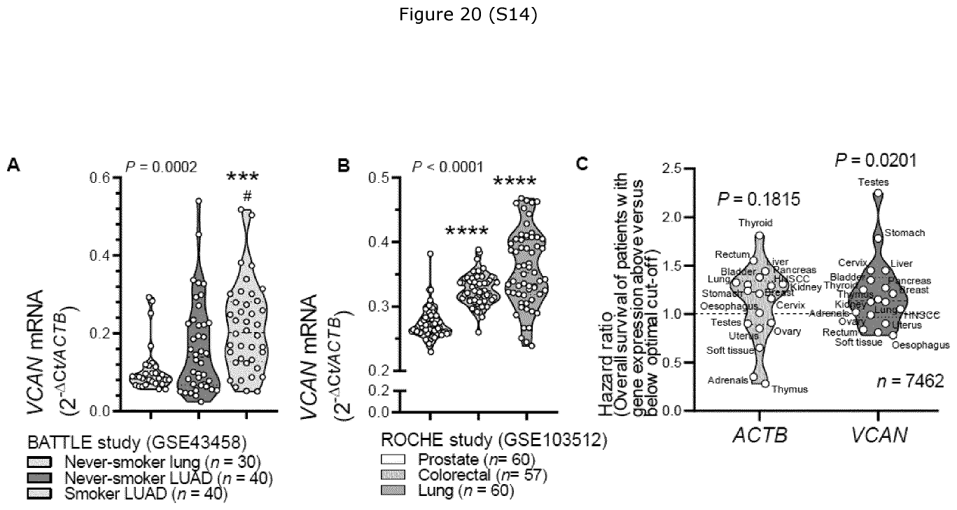

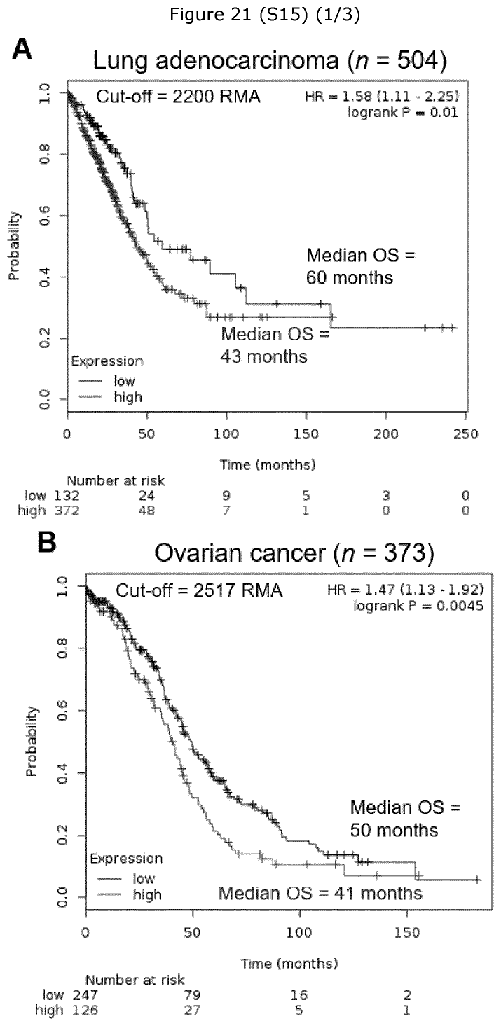

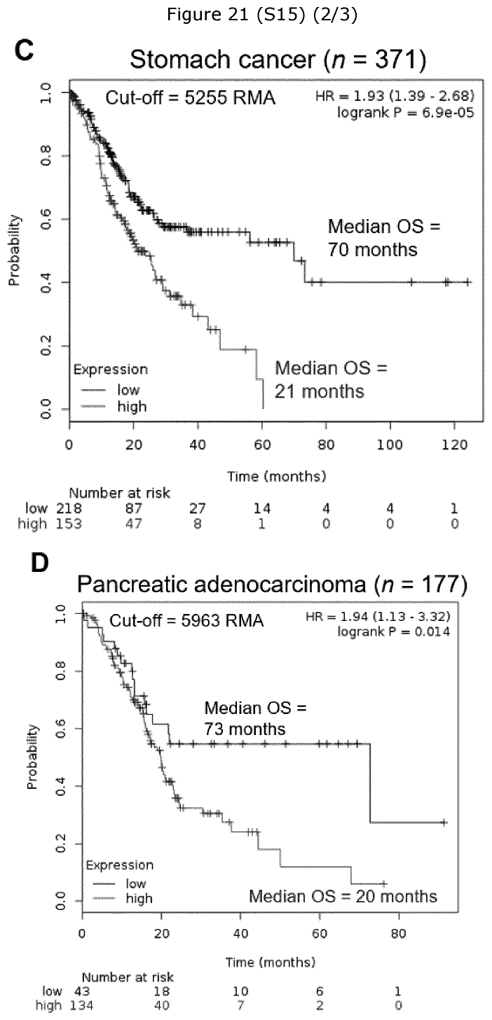

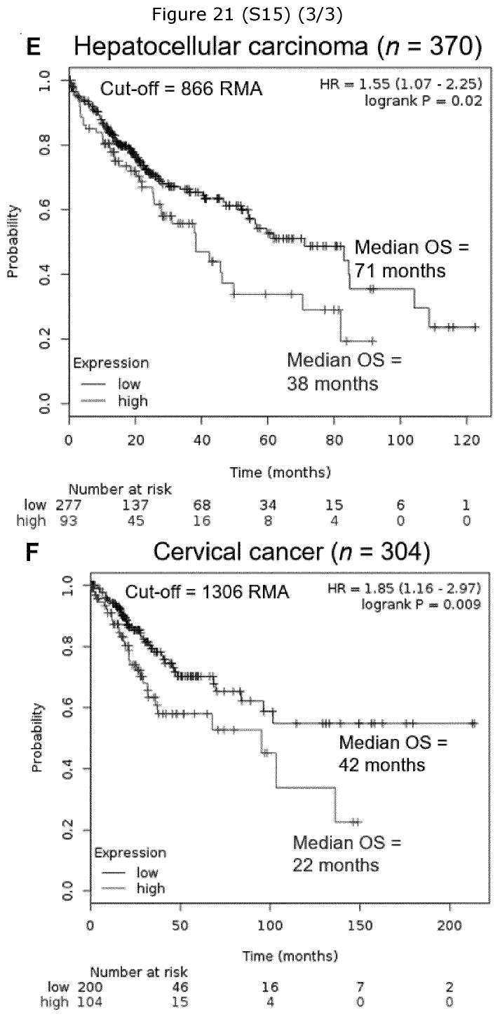

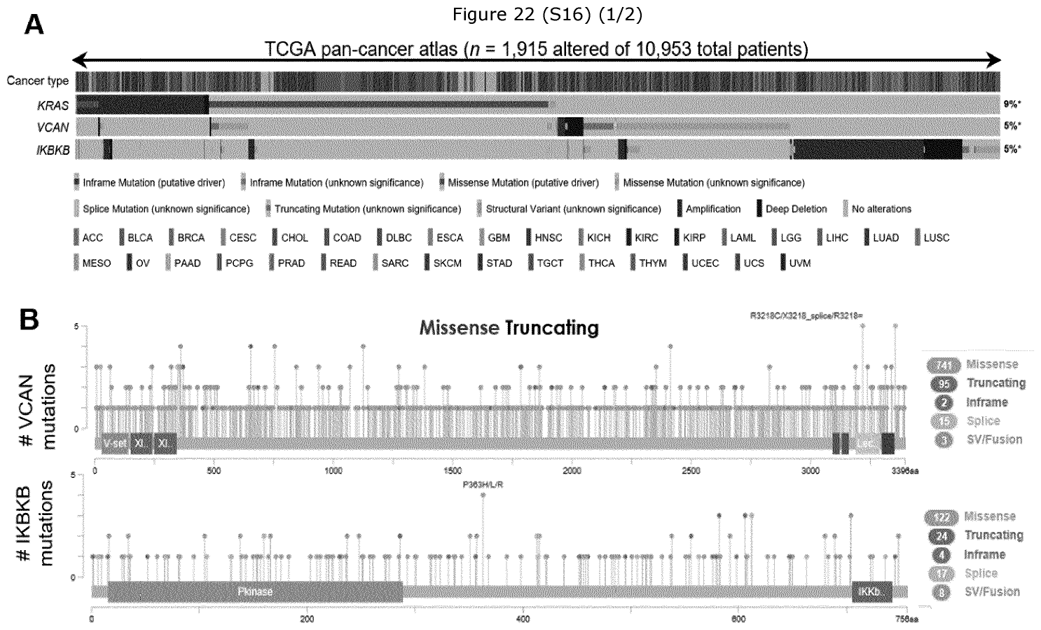

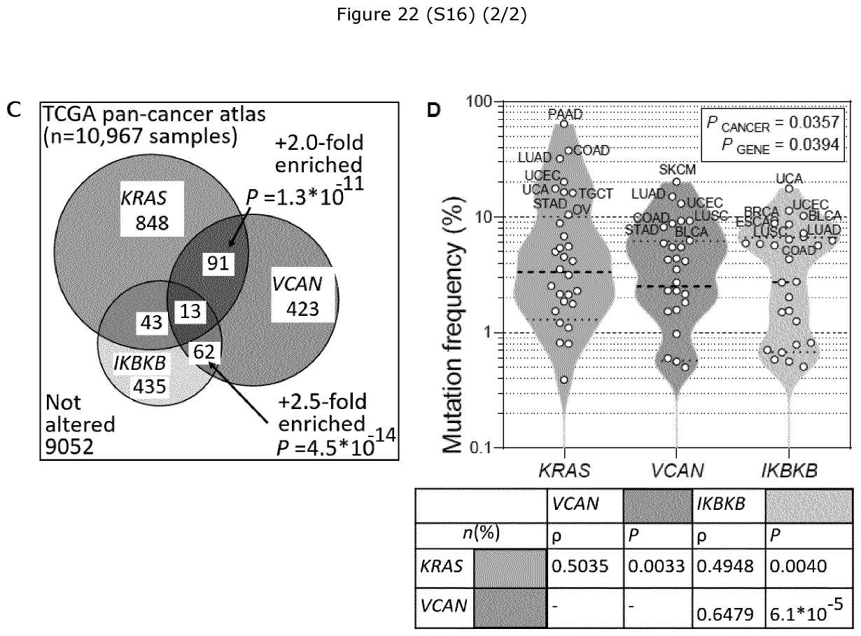

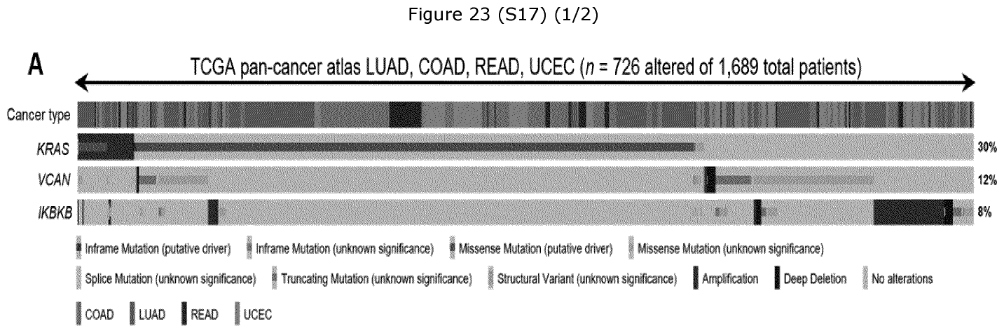

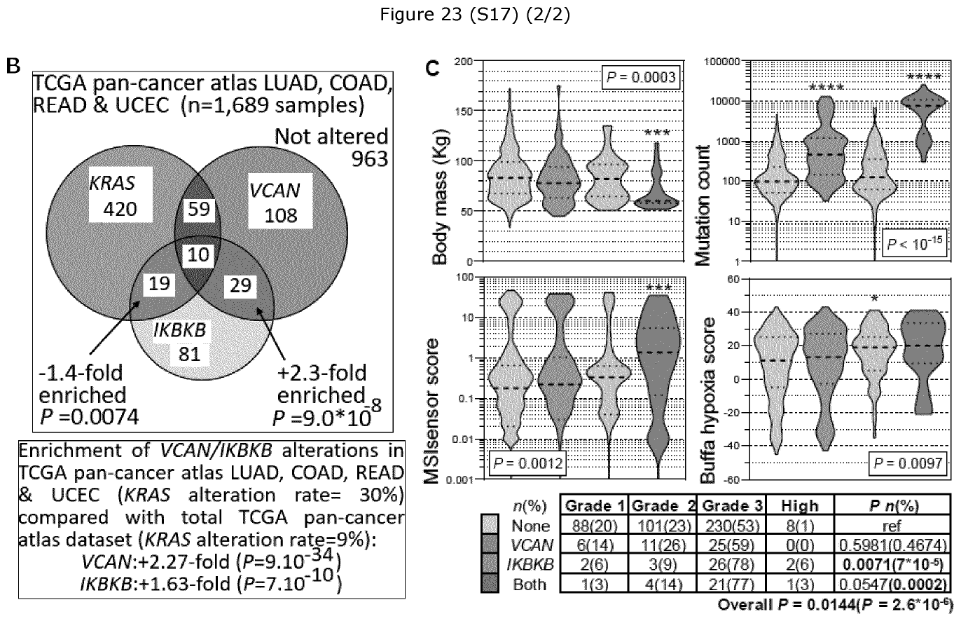

To block the proposed inflammatory loop, we employed the novel IL-1 receptor antagonist Isunakinra (30). Systemic delivery of Isunakinra to mice with already established tumors specifically inhibited s.c. growth of KrasMUT tumors (FIG. 6A). In addition, Isunakinra limited nuclear factor (NF)-κB activation in KrasMUT cancer cells in vivo, a phenomenon we previously showed to be fueled by myeloid IL-1β, as well as their ability for lethal MPE induction (FIGS. 6B-D). Since VCAN is a known TLR2 ligand (23), the proinflammatory loop proposed here was also targeted with the TLR1/2 inhibitor Cu-CPT22 (31). The drug effectively inhibited VCAN induced NF-κB activation and cellular survival in RAW264.7 macrophages at the low micromolar range and blocked tumor growth in vivo at clinically relevant concentrations (FIGS. 6E-H). Hence VCAN-IKKβ-mediated addiction of KRASMUT cancers to host IL-1β can be used to indirectly target these tumors.

### Discussion

Here we show how KRAS-mutant tumors are dependent on IL-β provided by tumor-associated macrophages. Importantly, we show that tumor-secreted versican causes IKKβ activation in myeloid cells to foster this pro-inflammatory circuitry. Notwithstanding cancers with other mutations and other myeloid cells like neutrophils and mast cells that might also fuel tumors with IL-1β, we define here a non-oncogene addiction of KRAS and IL-1β, in tandem with their partners in crime VCAN and IKKβ. The findings stress the need for molecular stratification of current clinical trials of IL-1β inhibition against lung cancer. Unique experimental models for the study of tumor genome-host immunity interactions are provided, and novel diagnostic platforms and prognostic biomarkers are described for further validation.

Although sotorasib was recently approved in the U.S. against KRASG12C-mutant NSCLC (32), KRAS-mutant cancers from multiple sites of origin remain notoriously aggressive and undruggable (33) and direct KRAS inhibition is associated with some toxicity that likely renders such treatments unsuitable for chemoprevention (34). On the contrary, anti-IL-1β-directed therapies hold promise for chemoprevention, as shown by the CANTOS trial, based on their excellent safety profile (2). We identify cancer cell VCAN and myeloid IKKβ as the accomplices of KRAS that trigger secretion of IL-1β in the milieu of KRAS-mutant cancers. The results position these cancers as favourite candidates for anti-IL-1β therapy, and versican as a diagnostic and prognostic biomarker, as well as a therapeutic target in this tumor category that comprises 9% of all human cancers, alone or in combination with anti-IL-1β agents.

NF-κB signaling in cancer and myeloid cells impacts modes of tumor progression and metastasis in various tumor types and is intimately addicted with oncogenic KRAS signaling (35, 36). However, the lessons learnt from clinical trials of proteasome (and hence also canonical NF-κB pathway) inhibitors against multiple myeloma dictate that therapeutic interventions into the NF-κB pathway are also associated with significant toxicity, since the pathway acts simultaneously in epithelial and immune cells in opposing fashions (37, 38). In addition to previous work elucidating the oncogenic functions of IKKβ in tumor cells (5, 10, 16, 29, 35, 36, 39), here we show how myeloid IKKβ functions to fuel tumor cell NF-κB signaling with IL-1β, further emphasizing the complex and multifaceted pro-tumor functions of NF-κB and the need for its therapeutic targeting against cancer.

In conclusion, KRAS-mutant cancers rely on host IL-1β, which they elicit from host macrophages via secretory versican that activates myeloid IKKβ. This inflammatory loop provides multiple opportunities for improved diagnosis, prognostication, and identification of therapeutic vulnerabilities of KRAS-mutant cancers.

### Materials and Methods

### Murine and Human Study Approval

All mice used for these studies were bred at the Department of Medicine of the University of Patras, Greece. Experiments were prospectively approved by the Veterinary Administration of the Prefecture of Western Greece (approval #276134/14873/2) and were conducted according to the European Union Directive 2010/63/EU (https://eur-lex.europa.eu/legalcontent/EN/TXT/?uri=celex % 3A32010L0063). Male and female experimental mice were sex-, weight (20-25 g)-, and age (6-12 weeks)-matched. Exact sample sizes (n) are included in the figures and their legends. Animals were assigned to experimental groups by randomization (when n≥20) or alternation (when n<20) with controls and experimental mice always being littermates, and transgenic animals enrolled case-control-wise. Data were collected by at least two blinded investigators from samples coded by non-blinded investigators. The Munich lung adenocarcinoma and Patras pleural effusion (13, 28) clinical studies were conducted in accord with the Helsinki Declaration (https://www.wma.net/policies-post/wma-declaration-of-helsinkiethical-principles-for-medical-research-involving-human-subjects/), were approved by the Ludwig-Maximilians-University Munich Ethics Committee (approval #623-15) and the University of Patras Ethics Committee (approval #22699/21.11.2013), were registered with the German Clinical Trials Register (Deutsches Register Klinischer Studien; #DRKS00012649; https://www.drks.de/drks_web/navigate.do?navigationId=trial. HTML&TRIAL_ID=DRKS00012649) and with ClinicalTrials.gov (Using pleural effusions to diagnose cancer; NCT03319472; https://clinicaltrials.gov/ct2/show/NCT03319472?term=NCT03319472&rank=1), respectively, and written informed consent was prospectively obtained from all patients.

### Reagents

D-Luciferin potassium salt [(S)-4,5-Dihydro-2-(6-hydroxy-2-benzothiazolyl)-4-thiazolecarboxylic acid potassium salt, Chemical Abstracts Service number, CAS #115144-35-9] was from Biosynth (Lake Constance, Switzerland). Clodronate (Dichloromethylenediphosphonic acid disodium salt, CAS #22560-50-5) and Hoechst 33258 nuclear dye (CAS #23491-45-4), were from Sigma-Aldrich (St. Louis, MO). Egg-phosphatidylcholine (CAS #97281-44-2) was from Avanti Polar Lipids (Alabaster, AL). Lentiviral shRNA, puromycin (CAS #58-58-2) and lipopolysaccharide (LPS; catalogue #sc-3535) were from Santa Cruz (Dallas, TX). Geneticin (G418; catalogue #10131035) was from Thermo Fisher Scientific (Waltham, MA). Recombinant human active versican (VCAN; catalogue #RPB817Mu01) and osteopontin (secreted phosphoprotein 1, SPP1; catalogue #APA899Hu61) were from Cloud-Clone Corp (Houston, TX) and all other recombinant proteins from Immunotools (Friesoythe, Germany). Bortezomib (CAS #179324-69-7) was from Selleckchem (Houston, TX). IL-1β ELISA (catalogue #900-K47) was from Peprotech (London, UK). Primers were from VBC Biotech (Vienna, Austria). Isunakinra (EBI-005), a recombinant protein that binds to the interleukin-1 receptor 1 (IL1R1) and potently blocks IL-1α and IL-1β beta (30), was from Buzzard Pharmaceutical (Stockholm, Sweden), and the TLR1/TLR2 antagonist Cu-CPT22 or 3,4,6-Trihydroxy-2-methoxy-5-oxo-5H-benzocycloheptene-8-carboxylic acid hexyl ester (CAS #1416324-85-0) (31) from Merck (Darmstadt, Germany). Primers and lentiviral shRNA pool sequences are listed in Tables 4 and 5 and antibodies in the respective methods sections.

### Cells

Lewis lung carcinoma (LLC, RRID:CVCL_4358), B16F10 skin melanoma (male, RRID:CVCL_0159), and PAN02 pancreatic adenocarcinoma cells (male, RRID:CVCL_D627) were from the National Cancer Institute Tumor Repository (Frederick, MD). RAW264.7 murine myelomonocytic leukaemia (male, RRID:CVCL_0493) and mouse lung epithelial 12 (MLE12, female, RRID:CVCL_3751) cells were from ATCC (Manassas, VA). MC38 colon adenocarcinoma (female, RRID:CVCL_B288) and AE17 mesothelioma (female, RRID:CVCL_4408) cells were gifts from Dr. Barbara Fingleton (Vanderbilt University, Nashville, TN) and Dr. Y. C. Gary Lee (University of Western Australia, Perth, Australia), respectively. FVB urethane-induced lung adenocarcinoma (FULA1) cells were produced in our laboratories (female, RRID:CVCL_A9 KV). Cells were cultured at 37° C. in 5% CO2-95% air using Dulbecco's modified Eagle's medium (DMEM) supplemented with 10% fetal bovine serum, 2 mM L-glutamine, 1 mM pyruvate, 100 U/mL penicillin, and 100 mg/mL streptomycin. For in vivo injections, cells were trypsinized, incubated with Trypan blue, counted with a grid hemacytometer according to the Neubauer method, and only 95% viable cells were used for experiments. All in vitro experiments were repeated independently at least three times and the stated n always reflects biological and not technical sample size. All cell lines have been repeatedly reported, were re-sequenced for Kras mutations and their status was verified to be the same as previously reported, and were tested annually for identity by short tandem repeats and for Mycoplasma Spp. by PCR using primers GGGAGCAAACAGGATTAGATACCCT (SEQ ID NO: 14) and TGCACCATCTGTCACTCTGTTAACCTC (SEQ ID NO:15) (amplicon size 270 bp) (9, 10, 15, 22, 29, 40).

### Experimental Mice

NGL and HLL NF-κB reporter mice are described elsewhere (20, 21, 39). Mice obtained from Jackson Laboratories (Bar Harbor, MN) were wild-type (WT) C57BL/6J mice (C57BL/6; #000664), B6.129(Cg)-Gt (ROSA) 26Sortm4 (ACTB-tdTomato,-EGFP) Luo/J dual membranous fluorescent Cre-recombinase reporter mice (mT/mG; #007676) (41), B6.129P2-Lyz2tm1 (cre) Ifo/J mice that express Cre-recombinase under control of the Lyz2 promoter (Lyz2.Cre; #004781) (22, 42), B6.129P2-Gt (ROSA) 26Sortm1 (DTA) Lky/J mice that express Diphtheria toxin upon Cre-mediated recombination that results in cell suicide (Dta; #009669) (22, 43), and B6;129S-Tnftm1Gkl/J Tnfdeficient mice (Tnf−/−; #005540) (10, 44). B6.B4B6-Chuk<tm1Mpa>/Cgn (Chukf/f) and B6.B4B6-Ikbkb<tm2.1Mpa>/Cgn (Ikbkbf/f) mice that carry conditional Chuk and Ikbkb alleles that are deleted upon Cre-recombinase expression (45, 46), as well as Il1btm1Yiw Il1b-deficient mice (Il1b−/−; MGI #215739631) (14) and Cpa3. Cre+/− mast cell-deficient mice in which mast cells undergo Trp53-mediated apoptosis (Cpa3.Cre) (47) were described elsewhere and were kindly donated by their founders. All mice used for these studies were originated from or back-crossed >F12 generations to the C57BL/6 background. For these studies, n=929 mice were used.

### Mouse Tumor Models

For generation of solid tumors, mice were injected subcutaneously (s.c.) in the shaven rear flank dermis with 5×10⁵ tumor cells in 100 μl of phosphate-buffered saline (PBS), as described elsewhere (9, 10, 15, 29). Mice were weekly examined for tumor volume (V) by measuring three vertical tumor diameters (d1, d2, d3) using the formula V=n*d1*d2*d3 and were killed when tumor volume reached 1 cm3 (PANO2 cells) or 2 cm3 (all other cell lines). For induction of malignant pleural effusions (MPE), mice received intrapleural injections of 2×10⁵ cancer cells suspended in 100 μL PBS and were sacrificed when showing signs of sickness or at the timepoints indicated (14-28 days post-tumor cell delivery depending on the cell line used) (9, 10, 15). In all models, both mice and inoculated cancer cells were always syngeneic to avoid inflammatory allograft rejection and artificial NF-κB activation.

### Bioluminescence and Biofluorescence Imaging

Mice were imaged for NF-κB reporter bioluminescent signal daily starting at day 10 post-tumor cell injection until sacrifice. For this, mice were anesthetized by isoflurane inhalation and were imaged for bioluminescence on a Xenogen Lumina II (Perkin-Elmer, Waltham, MA) 5-20 min after delivery of 1 mg D-Luciferin potassium salt diluted in 100 μL of sterile water into a retroorbital vein. Pleural tumors isolated from NGL mice were also imaged ex vivo for green biofluorescence using 410-440 nm background control excitation, 445-490 nm experimental excitation, and 515-575 nm emission passbands on a Xenogen Lumina II. Cells were imaged for bioluminescence on a Xenogen Lumina II 0, 4, 8, 16, and 24 hours after a single addition of 300 μg/mL (equivalent to 1 mM) D-luciferin to the culture media. Data were analyzed using Living Image v.4.2 (Perkin-Elmer, Waltham, MA) as described previously (9, 10, 15, 15, 22, 29, 39).

### Sequencing

Genomic DNA was extracted from cell lines using GenElute Mammalian Genomic DNA Miniprep Kit (Sigma-Aldrich). Kras exons 1-3 were amplified by PCR using Phusion Polymerase (New England Biolabs, Ipswich, MA) and 60° C. annealing temperature. Primers are described in Table 4. PCR products were analyzed on 1% agarose gels, purified by QIAquick Gel Extraction Kit (Qiagen, Hilden, Germany) and sequenced by Eurofins Genomics (Ebersberg, Germany).

### Constructs and Transfections

Control shRNA (shC, sc-108080-V; target sequences are proprietary of the manufacturer) and anti-mouse Vcan shRNA (shVcan, sc-41904-V) pools were from Santa Cruz. The pNGL construct and lentiviral shRNA pools for silencing of Kras, Chuk, Ikbkb, Ikbke, and Tbk1 were described previously (10). Lentiviral shRNA catalogue numbers and target sequences are listed in Table 5. For stable plasmid transfections, 105 RAW264.7 cells were transfected with 5 μg DNA using Xfect (Takara, Mountain View, CA), followed by selection by G418 (400-800 μg/mL). For stable shRNA transfection, 105 tumor cells were transfected with lentiviral particles, and clones were selected by puromycin (2-10 μg/mL) (9, 10).

### Intrapleural Catheter

For in vivo MPE drainage, a 1.2 cm-long catheter bearing serial fenestrations at 1-mm intervals was used, according to the detailed model description reported previously (48). Mice were anesthetized using isofluorane and the catheter insertion site was shaved and disinfected using 70% ethanol and 10% povidone iodide, and the catheter was then installed into the pleural space and sutured under the skin. Mice were imaged pre- and post-MPE drainage and were sacrificed thereafter.

### Cytology

MPE fluid was treated with red blood cell lysis buffer (155 mM NH4Cl, 12 mM NaHCO₃, 0.1 mM EDTA) and MPE cells were centrifuged and stained with May-Grunwald-Giemsa. Slides were then mounted with Entellan (Merck Millipore, Darmstadt, Germany) and microscopically analyzed for differential counting of pleural cells. Pleural lavage was performed by injecting 1 ml of saline intrapleurally and recovering it after 30 sec. Pleural cells were enumerated with a haemocytometer, were centrifuged, were stained with May-Grünwald-Giemsa or with anti-rabbit F4/80 antibody (ab111101; Abcam, London, UK; RRID:AB_10859466) and hematoxylin and were microscopically analyzed for differential counting of pleural cells.

### Flow Cytometry

Pleural effusion cells were treated with red blood cell lysis buffer (155 mM NH4Cl, 12 mM NaHCO₃, 0.1 mM EDTA), enumerated and 0.5-1.0×106 cells were processed for antibody staining. Pleural tumors were dissociated using 70 μm strainers (BD Bioscience, San Jose, CA), enumerated, and 0.5-1.0×106 cells were processed for antibody staining. BMDM were enumerated and 0.5-1.0×106 cells were processed for antibody staining. All samples were suspended in 50 μL PBS with 2% FBS and 0.1% NaN3, and stained with the following antibodies: anti-CD45 (11-0451-85; eBioscience, Santa Clara, CA; RRID:AB_465051), anti-CD11b (12-0112-82; eBioscience; RRID:AB_2734869), anti-Ly6C (45-5932-82; eBioscience; RRID:AB_2723343), anti-F4/80 (123128; Biolegend, San Diego, CA; RRID:AB_893484), anti-Ly6G (127624; Biolegend; AB_10640819), anti-GFP eFluor® 660 (50-6498-82; eBioscience; RRID:AB_11043268), anti-MHC Class II (17-5321; eBioscience; RRID:AB_469454), Alexa Fluor® 647 anti-CD206 (141712; Biolegend; RRID:AB_10900420), biotinylated anti-firefly Luciferase (ab634; Abcam, London, UK; RRID:AB_305434), and streptavidin (17-4317-82; eBioscience), for 20 min in the dark at a concentration of 0.1 μg/106 cells. Samples were analyzed on a CyFlowML flow cytometer using the FloMax Software (Partec, Darmstadt, Germany; RRID:SCR_014437), Flowing Software v.2.5.1 (http://flowingsoftware.btk.fi/; RRID:SCR_015781) and FlowJo Software v10.6.2 (BD Bioscience, San Jose, CA; RRID:SCR_008520).

### Immunohistochemistry

For dark field immunofluorescence, pleural tumors were fixed in 4% paraformaldehyde overnight at 4° C., cryoprotected with 30% sucrose, embedded in Tissue-Tek (Sakura, Tokyo, Japan) and stored at −80° C. Ten-μm cryosections were then post-fixed in 4% paraformaldehyde for 10 min, treated with 0.3% Triton X-100 for 5 min, blocked for 1 hour in 1× phosphate buffered saline (PBS) containing 10% fetal bovine serum (FBS), 3% bovine serum albumin (BSA), and 0.1% Tween 20, and then incubated with the indicated primary antibodies overnight at 4° C. Sections were subsequently treated with fluorescent secondary antibodies, counterstained with Hoechst 33258 (CAS #23491-45-4) and mounted with Mowiol 4-88 (Calbiochem, Darmstadt, Germany; CAS #9002-89-5). The following primary antibodies were used: mouse anti-GFP (1:200 dilution; sc-9996; Santa Cruz, Dallas, TX; RRID:AB_627695), rat anti-CD68:Alexa Fluor® 488 (MCA1957A488T; AbD Serotec, Kidlington, UK; RRID:AB_1102282), mouse anti-CD45 FITC (11-0451-85; eBioscience; RRID:AB_465051), and rabbit anti-PCNA (1:3000 dilution; ab18197; Abcam, London, UK; RRID:AB_444313). Alexa Fluor donkey anti-mouse 488 (A21202; RRID:AB_141607), Alexa Fluor goat anti-rat 568 (A11077; RRID:AB_141874), and Alexa Fluor donkey anti-rabbit 568 (A10042; RRID:AB_2534017) secondary antibodies used at 1:500 dilution were from Thermo Fisher Scientific (Waltham, MA). For isotype control, the primary antibody was omitted. Fluorescent microscopy was carried out either on an AxioObserver D1 inverted fluorescent microscope (Zeiss, Jena, Germany) or a TCS SP5 confocal microscope (Leica, Wetzlar, Germany) with 20×, 40×, and 63× lenses. Digital images were processed with Fiji academic freeware (RRID:SCR_002285) (49). All quantifications of cellular populations were obtained by counting at least five random non-overlapping tumor-containing fields of view per section. Bright field immunohistochemistry was done as described previously (9, 22, 29), and the following antibodies were used: rabbit anti-versican (1:100; E-AB-36300; Elabscience, Wuhan, China), mouse secondary anti-rabbit (1:5000; ab191866; Abcam, London, UK; RRID:AB_2650595). All quantifications of cellular populations were obtained by counting at least five random nonoverlapping tumor-containing fields of view per section.

### Bone Marrow Transfer (BMT) and Liposomal Clodronate

For adoptive BMT experiments described in detail elsewhere (9, 10, 15), wild-type (WT) and NF-κB.eGFP.LUC (NGL) recipient mice on the C57BL/6 background received total-body irradiation (1100 Rad) followed 12 hours later by 107 intravenous (via retro-orbital injection) whole bone marrow cells obtained from WT and NGL donors. One irradiated mouse per group was not transplanted with BMT to control for effective elimination of endogenous bone marrow and died 5-15 days post-irradiation. After one month, allowing for complete bone marrow reconstitution by chimeric bone marrow cells, liposomal clodronate was prepared as described previously (21, 50) and 500 μg were administered intrapleurally. After yet another month required for replacement of pleural myeloid cells by transplanted bone marrow cells (50), mice were injected with tumor cells.

### Bone Marrow Derived Macrophages (BMDM)

For BMDM generation, 107 bone marrow cells were plated and cultured for seven days in the presence of 100 ng/ml macrophage colony stimulating factor (M-CSF). Where appropriate, at day 6 of the culture, recombinant human versican (1 nM) was added to the culture medium or, alternatively, the culture medium was removed and BMDM were exposed to cancer cell-conditioned media for 4 hours. Culture supernatants were then isolated for ELISA and cells were processed for western blot, flow cytometry, or qPCR.

### Immunoblotting

Nuclear and cytoplasmic protein extracts were prepared using the NEPER Extraction Kit (Thermo Fisher Scientific, Waltham, MA), separated by SDS-PAGE and electroblotted to PVDF membranes (Merck Millipore, Darmstadt, Germany). Membranes were probed with the following primary antibodies: anti-IKKα (1:1000 dilution; 2682; Cell Signaling, Danvers, MA; dilution; 2684; Cell Signaling; RRID:AB_331626), anti-IKKβ (1:1000 RRID:AB_2122298), anti-VCAN (1:200 dilution; ab19345; Abcam, London, UK; RRID:AB_444865), anti-β-actin (1:500 dilution; sc-47778; Santa Cruz, Dallas, TX; RRID:AB_2714189), and anti-α-tubulin (TUBA; 1:4000 dilution; T5168; Sigma Aldrich, St. Louis, MO; RRID:AB_477579), followed by incubation with secondary goat anti-mouse (1:8000 dilution; 1030-05; Southern Biotech, Birmingham, AL; RRID:AB_2619742) or goat anti-rabbit (1:8000 dilution; 4030-05; Southern Biotech; RRID:AB_2687483) HRP-conjugated antibodies. Membranes were visualized by chemiluminescent film exposure after incubation with enhanced chemiluminescence substrate (Merck Millipore, Darmstadt, Germany).

qPCR and Microarrays

Triplicate cultures of 10⁶ cells were subjected to RNA extraction using Trizol (Thermo Fisher Scientific, Waltham, MA) followed by column purification and DNA removal (RNeasy Mini Kit, Qiagen, Hilden, Germany). Pooled RNA (5 μg) was quality tested on an ABI 2000 bioanalyzer (Agilent Technologies, Sta. Clara, CA), labelled, and hybridized to GeneChip Mouse Gene 2.0 ST arrays (Affymetrix, Sta. Clara, CA). All data were analyzed on the Affymetrix Expression and Transcriptome Analysis Consoles (RRID:SCR_018718). RNA was reverse transcribed with Superscript III (Thermo Fisher Scientific) and qPCR was performed using first strand synthesis and SYBR FAST qPCR Kit (Kapa Biosystems, Wilmington, MA) in a StepOne cycler (Applied Biosystems, Carlsbad, CA). Primers for qPCR are listed in Table 4. Ct values from triplicate reactions were analyzed with the relative quantification method 2-ACT relative to mouse Gusb or human ACTB transcripts (51).

### Shotgun Proteomics

Supernatants obtained from murine KrasMUT (LLC, MC38, AE17) and KrasWT (B16F10 and PANO2) cell cultures (pooled triplicate cultures for each cell line; five million cells/175 cm² culture flask/24 hours in full DMEM followed by 24 hours in FBS-free DMEM) were analyzed. For this, 600 μL of cell culture supernatant were enzymatically digested using a modified filteraided sample preparation (FASP) protocol (52, 53). Peptides were stored at −20° C. until mass spectrometry (MS) measurements. MS data were acquired in data-dependent acquisition (DDA) mode on a Q Exactive (QE) high field (HF) mass spectrometer (Thermo Fisher Scientific). Approximately 0.5 μg per sample were automatically loaded to the online coupled RSLC (Ultimate 3000, Thermo Fisher Scientific) HPLC system. A nano trap column was used (300 μm inner diameter (ID)×5 mm, packed with Acclaim PepMap100 C18, 5 μm, 100 Å (LC Packings, Sunnyvale, CA) before separation by reversed phase chromatography (Acquity UPLC M-Class HSS T3 Column 75 μm ID×250 mm, 1.8 μm; Waters, Eschborn, Germany) at 40° C. Peptides were eluted from 3% to 40% over a 95 minute gradient. The MS spectrum was acquired with a mass range from 300 to 1500 m/z at resolution 60 000 with AGC set to 3×10⁶ and a maximum of 50 ms IT. From the MS prescan, the 10 most abundant peptide ions were selected for fragmentation (MSMS) if at least doubly charged, with a dynamic exclusion of 30 seconds. MSMS spectra were recorded at 15 000 resolution with AGC set to 1×10⁵ and a maximum of 100 ms IT. CE was set to 28 and all spectra were recorded in profile type. Label-free quantification of DDA-MS data was performed with Proteome discoverer (version 2.3; Thermo Fisher Scientific) using Sequest HT (as node in PD) and searching against the UniProtKB/Swiss-Prot Mouse database (release 2017_2, 16872 sequences). Searches were performed with a precursor mass tolerances of 10 ppm and fragment mass tolerances of 0.02 Da. Carbamidomethylation (C) was set as static modification, deamidation (N,Q), oxidation (M), and N-terminal Met-loss+Acetyl were selected as dynamic modifications and two missed cleavages were allowed. Percolator (54) was used for validating peptide spectrum matches and peptides, accepting only the top-scoring hit for each spectrum, and satisfying the cut-off values for FDR<1%, and posterior error probability <0.01. The final list of proteins complied with the strict parsimony principle. The quantification of proteins, after precursor recalibration, was based on abundance values (area under curve) for unique peptides. Abundance values were normalized in a retention time dependent manner. The protein abundances were calculated summing the abundance values for admissible peptides. Comparisons between KrasMUT (LLC, MC38, AE17) and KrasWT (B16F10 and PANO2) cell lines were done using only the proteins detected in all five cell lines.

### Cellular Treatments

Cells were exposed to tumor-conditioned media diluted 1:1 in DMEM. Bortezomib pre-treatment was applied 1 hour prior to exposure to conditioned media at 1 μg/mL (equivalent to 3 μM). Cells were exposed to potential NF-κB ligands at the following concentrations: lipopolysaccharide, LPS, 1 μg/mL (equivalent to 10-20 nM); secreted phosphoprotein 1, SPP1, 100 ng/ml (equivalent to 1.25-2.5 nM); tumor necrosis factor, TNF, 20 ng/ml (equivalent to 1 nM); versican, VCAN, 360 ng/ml (equivalent to 1 nM); interleukin (IL)-1β, 30 ng/ml (equivalent to 1 nM); and C—C-motif chemokine ligand 2, CCL2, 20 ng/ml (equivalent to 1.5 nM) and were imaged for bioluminescence or processed for other assays after 4 hours. pNGL RAW264.7 macrophages were exposed to 1 nM VCAN followed by treatment with increasing concentrations of TLR1/TLR2 antagonist Cu-CPT22.

### Mouse Treatments

The IL-1 receptor antagonist isunakinra (30) was given via daily intraperitoneal injections of 20-50 mg/Kg drug diluted in 100 μL PBS. Therapy was initiated at 10-17 days post s.c. tumor cells or at 5 days post-intrapleural tumor cells, allowing for efficient tumor take and a therapeutic study design. Treatment with the TLR1/TLR2 antagonist Cu-CPT22 (31) was initiated 3 days after intrapleural cancer cell injection and consisted of daily intraperitoneal injections of 100 μl corn oil 10% DMSO or 20 mg/kg Cu-CPT22 diluted in 100 μl corn oil 10% DMSO.

### Data Availability

Survival data were obtained from the Kaplan-Meier plotter pan-cancer RNAseq dataset (https://kmplot.com/analysis/) using search term VCAN. TCGA pan-cancer data were downloaded from https://www.cbioportal.org/.

### Transcription Factor Binding Site Analyses

We downloaded the RELA and RELB binding sequence motifs from the ENCODE portal (https://www.encodeproject.org/) with the identifiers: ENCFF507YCV (CHIP-seq on HuH-7.5 cells) and ENCFF615HZF (CHIP-seq on 8988T cells), respectively, and queried the ChIPseq datasets from the ChIP-X Enrichment Analysis (CHEA) Transcription Factor Targets dataset (https://maayanlab.cloud/Harmonizome/dataset/CHEA+Transcription+Factor+Targets) (55, 56).

### Statistics

Sample size was calculated using power analysis on G*power (57), assuming α=0.05, β=0.05, and effect size d=1.5. No data were excluded from analyses. Pooled data from repeated in vivo experiments are shown. All in vitro experiments were repeated independently at least three times and the stated n always reflects biological and not technical sample size. Animals were allocated to treatments by randomization (when n≥20) or alternation (when n<20) and transgenic animals were enrolled case-control-wise. Data were collected by at least two blinded investigators from samples coded by non-blinded investigators. All data were tested for normality of distribution by Kolmogorov-Smirnov test, are given as violin plots or mean±SD, and sample size (n) always refers to biological and not technical replicates. Differences in frequency were examined by Fischer's exact and χ² tests, in medians by Mann-Whitney or Kruskal-Wallis test with Dunn's post-tests, and in means by t-test or one-way ANOVA with Bonferroni post-tests. Changes over time and interaction between two variables were examined by two-way ANOVA with Bonferroni post-tests. Hypergeometric tests were done at the Graeber Lab website (https://systems.crump.ucla.edu/hypergeometric/index.php). All probability (P) values are two-tailed and were considered significant when P<0.05. All analyses and plots were done on Prism v8.0 (GraphPad, La Jolla, CA; RRID:SCR_002798).

TABLE 4
PCR primers used in this study.

| | | | | SEQ |
|---|---|---|---|---|
| | | | Product | ID |
| Assay^{a} | Primer | Sequence | (bp) | NO: |
| PCR | Kras exon | TCTTAGAGTTTTACACACAAAGGT | 289 | 8 |
| | 1F | GAG | | |
| PCR | Kras exon | TTCAAAGCGGCTGGCTGC | | 9 |
| | 1R | | | |
| PCR | Kras exon | TTGAGCTGTTTACATCACCTTG | 315 | 10 |
| | 2F | | | |
| PCR | Kras exon | GCAAAGAATCAATAAATGTAAGCT | | 11 |
| | 2R | ATC | | |
| PCR | Kras exon | GTTGAGGGCAAGGTTTGAAG | 287 | 12 |
| | 3F | | | |
| PCR | Kras exon | ACGAACACCATCTGGAGCAC | | 13 |
| | 3R | | | |
| PCR | Mycoplasma | GGGAGCAAACAGGATTAGATACCC | 270 | 14 |
| | Spp. F | T | | |
| PCR | Mycoplasma | TGCACCATCTGTCACTCTGTTAACC | | 15 |
| | Spp. R | TC | | |
| qPCR | mVcan F | CCAGAACGGAAATATCAAGATTGG | 201 | 16 |
| qPCR | mVcan R | TGAAACACAACACCATCCAC | | 17 |
| qPCR | mIl1b F | TTTGACAGTGATGAGAATGACC | 162 | 18 |
| qPCR | mIl1b R | AATGAGTGATACTGCCTGCC | | 19 |
| qPCR | mTnf F | GTCCCCAAAGGGATGAGAAGT | 124 | 20 |
| qPCR | mTnf R | TTTGCTACGACGTGGGCTAC | | 21 |
| qPCR | mGusb F | TTACTTTAAGACGCTGATCACC | 165 | 22 |
| qPCR | mGusb R | ACCTCCAAATGCCCATAGTC | | 23 |
| qPCR | hACTB F | GAGCACAGAGCCTCGCCTTT | 70 | 24 |
| qPCR | hACTB R | TCATCATCCATGGTGAGCTGG | | 25 |
| qPCR | hVCAN F | GAATGTCACTCTAATCCCTGTC | 117 | 26 |
| qPCR | hVCAN R | TGTCTCGGTATCTTGCTCAC | | 27 |

ªAssay: PCR, DNA polymerase chain reaction; qPCR, quantitative (real-time) PCR. Provider: VBC Biotech, Vienna, Austria.

TABLE 5
Lentiviral shRNA pools used in this study.

| | | | | SEQ |
|---|---|---|---|---|
| | | Catalog | | ID |
| Target | Abbreviation | # | Target Sequences | NO: |
| random | shC | SC- | target sequence proprietary | |
| | | 108080- | | |
| | | V | | |
| Vcan | shVcan | SC- | 5′-CAACCACCATCGAAATGAA-3′ | 28 |
| | | 41904- | 5′-CTCACAGGCTGTAATTACA-3′ | 29 |
| | | V | 5′-GTAGACATATCCCATACTA-3′ | 30 |
| Kras | shKras | SC- | 5′-CTACAGGAAACAAGTAGTA-3′ | 31 |
| | | 43876- | 5′-GAACAGTAGACACGAAACA-3′ | 32 |
| | | V | 5′-CCATTCAGTTTCCATGTTA-3′ | 33 |
| Chuk | shChuk | SC- | 5′-CCATGGTGTTTGAATGTATTT-3′ | 34 |
| | | 29366- | 5′-CTCTCAGTGTGTTCTAGATTT-3′ | 35 |
| | | V | 5′-GCAAGCAGAAGATTATTGATT-3′ | 36 |
| Ikbkb | shIkbkb | SC- | 5′-GATGACATCTTGAACTTGATT-3′ | 37 |
| | | 35645- | 5′-CTGCACATTTGAATCTGTATT-3′ | 38 |
| | | V | 5′-CAGCTCTCTTAGACAGTTATT-3′ | 39 |
| Ikbke | shIkbke | SC- | 5′-GAGATCATGTACAGAATCATT-3′ | 40 |
| | | 39057- | 5′-CAGTGTTGTTTGGACAAGATT-3′ | 41 |
| | | V | 5′-CCAACAAACTAGCATTACTTT-3 | 42 |
| Tbk1 | shTbk1 | SC- | 5′-GTAGGACTGAGATATGAAATT-3′ | 43 |
| | | 39059- | 5′-GCATCACAGAGATTTACTATT-3′ | 44 |
| | | V | 5′-GAAGTTCTAGTTTGCACAATT-3′ | 45 |

### Provider: Santa Cruz Biotechnology, Dallas, TX.

### REFERENCES

1. C. I. Diakos, et al., Cancer-related inflammation and treatment effectiveness. Lancet Oncol. 15, e493-503 (2014). doi: 10.1016/S1470-2045 (14) 70263-3.
2. P. M. Ridker, et al., CANTOS Trial Group, Effect of interleukin-1beta inhibition with canakinumab on incident lung cancer in patients with atherosclerosis: exploratory results from a randomised, double-blind, placebo-controlled trial. Lancet 390, 1833-1842 (2017). doi: 10.1016/S0140-6736 (17) 32247-X.
3. N. A. Rizvi, et al., Cancer immunology. Mutational landscape determines sensitivity to PD-1 blockade in non-small cell lung cancer. Science 348, 124-128 (2015). doi: 10.1126/science.aaa1348.
4. F. Skoulidis, et al., STK11/LKB1 mutations and PD-1 inhibitor resistance in KRAS-mutant lung adenocarcinoma. Cancer Discov. 8, 822-835 (2018). doi: 10.1158/2159-8290.CD-18-0099.
5. L. Seguin, et al., An integrin beta (3)-KRAS-RalB complex drives tumour stemness and resistance to EGFR inhibition. Nat. Cell Biol. 16, 457-468 (2014). doi: 10.1038/ncb2953.
6. L. Soucek, E. R. et al., Mast cells are required for angiogenesis and macroscopic expansion of Myc-induced pancreatic islet tumors. Nat. Med. 13, 1211-1218 (2007). doi: 10.1038/nm1649.
7. D. Bang, et al., GSK-3alpha promotes oncogenic KRAS function in pancreatic cancer via TAK1-TAB stabilization and regulation of noncanonical NF-kappaB. Cancer Discov. 3, 690-703 (2013). doi: 10.1158/2159-8290.CD-12-0541.
8. J. Ling, et al., KrasG12D-induced IKK2/beta/NFkappaB activation by IL-1alpha and p62 feedforward loops is required for development of pancreatic ductal adenocarcinoma. Cancer Cell 21, 105-120 (2012). doi: 10.1016/j.ccr.2011.12.006.
9. T. Agalioti, et al. Mutant KRAS promotes malignant pleural effusion formation. Nat. Commun. 8, 15205 (2017). doi: 10.1038/ncomms15205.
10. A. Marazioti, et al., Myeloid-derived interleukin-1beta drives oncogenic KRAS-NF-kappaB addiction in malignant pleural effusion. Nat. Commun. 9, 672 (2018). doi: 10.1038/s41467-018-03051-z.
11. C. C. Wong, et al., Inhibition of IL1B by canakinumab may be effective against diverse molecular subtypes of lung cancer: an exploratory analysis of the CANTOS trial. Cancer Res. 80, 5597-5605 (2020). doi: 10.1158/0008-5472.CAN-19-3176.
12. J. D. Campbell, et al. Cancer Genome Atlas Research Network; M. N. Artyomov, R. Schreiber, R. Govindan, M. Meyerson, Distinct patterns of somatic genome alterations in lung adenocarcinomas and squamous cell carcinomas. Nat. Genet. 48, 607-616 (2016). doi: 10.1038/ng.3564.
13. L. V. Klotz, et al. Comprehensive clinical profiling of the Gauting locoregional lung adenocarcinoma donors. Cancer Med. 8, 1486-1499 (2019). doi: 25 10.1002/cam4.2031.
14. R. Horai, M. et al., Production of mice deficient in genes for interleukin (IL)-1alpha, IL-1beta, IL-1alpha/beta, and IL-1 receptor antagonist shows that IL-1beta is crucial in turpentine-induced fever development and glucocorticoid secretion. J. Exp. Med. 187, 1463-1475 (1998).
15. A. D. Giannou, et al. Mast cells mediate malignant pleural effusion formation. J. Clin. Invest. 125, 2317-2334 (2015). doi: 10.1172/JCI79840.
16. A. G. McLoed, et al. Neutrophil-derived IL-1beta impairs the efficacy of NF-kappaB inhibitors against lung cancer. Cell Rep. 16, 120-132 (2016). doi: 10.1016/j.celrep.2016.05.085.
17. C. Voigt, et al. Cancer cells induce interleukin-22 production from memory CD4+ T cells via interleukin-1 to promote tumor growth. Proc. Natl. Acad. Sci. U.S.A. 114, 12994-12999 (2017). doi: 10.1073/pnas. 1705165114.
18. J. Hiscott, et al. Characterization of a functional NF-kappa B site in the human interleukin 1 beta promoter: evidence for 5 a positive autoregulatory loop. Mol. Cell. Biol. 13, 6231-40 (1993). doi: 10.1128/mcb.13.10.6231-6240.1993.
19. K. Manthiram, et al. The monogenic autoinflammatory diseases define new pathways in human innate immunity and inflammation. Nat. Immunol. 18, 832-842 (2017). doi: 10.1038/ni.3777.
20. T. S. Blackwell, et al. Multiorgan nuclear factor kappa B activation in a transgenic mouse model of systemic inflammation. Am. J. Respir. Crit. Care Med. 162, 1095-1101 (2000). doi: 10.1164/ajrccm.162.3.9906129.
21. M. B. Everhart, et al. Duration and intensity of NF-kappaB activity determine the severity of endotoxin-induced acute lung injury. J. Immunol. 176, 4995-5005 (2006). doi: 10.4049/jimmunol. 176.8.4995.
22. M. Spella, et al. Club cells form lung adenocarcinomas and maintain the alveoli of adult mice. Elife. 8, e45571 (2019). doi: 10.7554/eLife.45571.
23. S. Kim, et al. Carcinoma-produced factors activate myeloid cells through TLR2 to stimulate metastasis. Nature 457, 102-106 (2009). doi: 10.1038/nature07623.
24. M. Kabbout, et al. ETS2 mediated tumor suppressive function and MET oncogene inhibition in human non-small cell lung cancer. Clin. Cancer Res. 19, 3383-3395 (2013). doi: 10.1158/1078-0432.CCR-13-30 0341.
25. J. Brouwer-Visser, et al. Regulatory T-cell Genes Drive Altered Immune Microenvironment in Adult Solid Cancers and Allow for Immune Contextual Patient Subtyping. Cancer Epidemiol. Biomarkers Prev. 27, 103-112 (2018). doi: 10.1158/1055-9965.EPI-17-0461.
26. S. A. Forbes, et al. COSMIC: High-Resolution Cancer Genetics Using the Catalogue of Somatic Mutations in Cancer. Curr. Protoc. Hum. Genet. 91, 10.11.1-10.11.37 (2016). doi: 10.1002/cphg.21.
27. Á. Nagy, et al. Validation of miRNA prognostic power in hepatocellular carcinoma using expression data of independent datasets. Sci. Rep. 8, 9227 (2018). doi: 10.1038/s41598-018-27521-y.
28. A. Marazioti, et al. Clinical identification of malignant pleural effusions. medRxiv. 2020.05.31.20118307. doi: https://doi.org/10.1101/2020.05.31.20118307.
29. M. Vreka, et al. IkappaB Kinase alpha Is Required for Development and Progression of KRAS-Mutant Lung Adenocarcinoma. Cancer Res. 78, 2939-2951 (2018). doi: 10.1158/0008-5472.CAN-17-1944.
30. J. Hou, et al. Design of a superior cytokine antagonist for topical ophthalmic use. Proc. Natl. Acad. Sci. U.S.A. 110, 3913-3918 (2013). doi: 10.1073/pnas.1217996110.
31. K. Cheng, et al. Discovery of small-molecule inhibitors of the TLR1/TLR2 complex. Angew. Chem. Int. Ed. Engl. 51, 12246-12249 (2012). doi: 10.1002/anie.201204910.
32. Sotorasib Edges Closer to Approval. Cancer Discov. 11, OF2 (2021). doi: 10.1158/2159-8290.CD-NB2021-0309.
33. A. G. Stephen, et al., Dragging ras back in the ring. 20 Cancer Cell 25, 272-281 (2014). doi: 10.1016/j.ccr.2014.02.017.
34. D. S. Hong, et al. KRASG12C Inhibition with Sotorasib in Advanced Solid Tumors. N. Engl. J. Med. 383, 1207-1217 (2020). doi: 10.1056/NEJMoa1917239.
35. Greten F R, et al. IKKbeta links inflammation and tumorigenesis in a mouse model of colitis associated cancer. Cell. 2004; 118 (3): 285-296.
36. Yang J, et al. Myeloid IKKbeta promotes antitumor immunity by modulating CCL11 and the innate immune response. Cancer Res. 2014; 74 (24): 7274-7284.
37. S Miyakoshi, et al. Severe pulmonary complications in Japanese patients after bortezomib treatment for refractory multiple myeloma. Blood 107, 35 3492-3494 (2006). doi: 10.1182/blood-2005-11-4541.
38. P. Moreau et al., Once weekly versus twice weekly carfilzomib dosing in patients with relapsed and refractory multiple myeloma (A.R.R.O.W.): interim analysis results of a randomised, phase 3 study. Lancet Oncol. 19, 40 953-964 (2018). doi: 10.1016/S1470-2045 (18) 30354-1.
39. G. T. Stathopoulos, et al. Epithelial NF-kappaB activation promotes urethane-induced lung carcinogenesis. Proc. Natl. Acad. Sci. U.S.A. 104, 18514-18519 (2007). doi: 10.1073/pnas.0705316104.
40. N. I. Kanellakis, et al. Tobacco chemical-induced mouse lung adenocarcinoma cell lines pin the prolactin orthologue proliferin as a lung tumour promoter. Carcinogenesis 40, 1352-1362 (2019). doi: 10.1093/carcin/bgz047.
41. M. D. Muzumdar, et al. A global double-fluorescent Cre reporter mouse. Genesis 45, 593-605 (2007). doi: 10.1002/dvg.20335.
42. B. E. Clausen, et al. Conditional gene targeting in macrophages and granulocytes using LysMcre mice. Transgenic Res. 8, 265-277 (1999). doi: 10.1023/a: 1008942828960.
43. D. Voehringer, et al. Homeostasis and effector function of lymphopenia-induced “memory-like” T cells in constitutively T cell-depleted mice. J. Immunol. 180, 4742-4753 (2008). doi: 10.4049/jimmunol.180.7.4742.
44. M. Pasparakis, et al. Immune and inflammatory responses in TNF alpha-deficient mice: a critical requirement for TNF alpha in the formation of primary B cell follicles, follicular dendritic cell networks and germinal centers, and in the maturation of the humoral immune response. J. Exp. Med. 184, 1397-1411 (1996). doi: 20 10.1084/jem.184.4.1397.
45. R. Gareus, et al. Normal epidermal differentiation but impaired skin-barrier formation upon keratinocyte-restricted IKK1 ablation. Nat. Cell Biol. 9, 461-469 (2007). doi: 10.1038/ncb1560.
46. M. Pasparakis, et al. TNFmediated inflammatory skin disease in mice with epidermis-specific deletion of IKK2. Nature 417, 861-866 (2002). doi: 10.1038/nature00820.
47. T. B. Feyerabend, et al. Cre-mediated cell ablation contests mast cell contribution in models of antibody- and T cell-mediated autoimmunity. Immunity 35, 832-844 (2011). doi: 10.1016/j.immuni.2011.09.015.
48. C. Merrick, et al. Novel mouse model of indwelling pleural catheter in mice with malignant pleural effusion. ERJ. Open Res. 5, 00226-2018 (2019). doi: 10.1183/23120541.00226-2018.
49. J. Schindelin, et al., Fiji: an open-source platform for biological-image analysis. Nat. 40 Methods. 9, 676-682 (2012). doi: 10.1038/nmeth.2019.
50. M. B. Everhart, et al. Intratracheal administration of liposomal clodronate accelerates alveolar macrophage reconstitution following fetal liver transplantation. J. Leukoc. Biol. 77, 173-180 (2005). doi: 10.1189/jlb.1203647.
51. M. W. Pfaffl, A new mathematical model for relative quantification in real-time RT-PCR. Nucleic Acids Res. 29, e45 (2001). doi: 10.1093/nar/29.9.e45.
52. J. R. Wiśniewski, et al. Universal sample preparation method for proteome analysis. Nat. Methods. 6, 359-362 (2009). doi: 10.1038/nmeth.1322.
53. A. Grosche, et al. The Proteome of Native Adult Muller Glial Cells From Murine Retina. Mol. Cell. Proteomics. 15, 462-480 (2016). doi: 10.1074/mcp.M115.052183.
54. L. Käll, et al. Semi-supervised learning for peptide identification from shotgun proteomics datasets. Nat. Methods. 4, 923-925 (2007). doi: 10.1038/nmeth1113.
55. ENCODE Project Consortium, The ENCODE (ENCyclopedia Of DNA Elements) Project. Science 306, 636-640 (2004). doi: 10.1126/science.1105136.
56. A. Lachmann, et al., ChEA: transcription factor regulation inferred from integrating genome-wide ChIP-X experiments. Bioinformatics 26, 2438-2444 (2010). doi: 10.1093/bioinformatics/btq466.
57. F. Faul, et al. G*Power 3: a flexible statistical power analysis program for the social, behavioral, and biomedical sciences. Behav. Res. Methods. 20 39, 175-191 (2007). doi: 10.3758/bf03193146.

1. A method of treating a cancer in a subject, the method comprising administering to the subject an inhibitor of interleukin-1 receptor type 1 (IL-1R1) signalling.

2. The method according to claim 1, wherein the cancer has been determined as being one comprising a KRAS mutation.

3. The method according to claim 1, wherein the cancer comprises an elevated level of VCAN mRNA and/or VCAN protein.

4. The method according to claim 1, wherein the elevated level and/or activity of VCAN is a level and/or activity of VCAN that is elevated relative to the level and/or activity of VCAN in a reference sample.

5. The method according to claim 1, wherein the cancer comprises an elevated level and/or activity of IL-1β.

6. The method according to claim 5, wherein the cancer comprises an elevated level of IL-1β mRNA and/or IL-1β protein.

7. The method according to claim 5, wherein the elevated level and/or activity of IL-1β is a level and/or activity of IL-1β that is elevated relative to the level and/or activity of IL-1β level in a reference sample.

8. The method according to claim 4, wherein the reference sample is a non-cancerous sample.

9. The method according to claim 8, wherein the non-cancerous sample is a sample comprising non-cancerous cells or tissue from a subject.

10. The method according to claim 8, wherein the non-cancerous sample is from the same subject or a different subject.

11. The method according to claim 1, wherein the cancer is selected from the group comprising lung cancer (such as non-small cell lung cancer (NSCLC)), kidney cancer, skin cancer, thymus cancer, breast cancer, pancreatic cancer, thyroid cancer, bladder cancer, liver cancer, cervical cancer, endometrial cancer, colorectal cancer, and stomach cancer.

12. The method according to claim 1, wherein the cancer is selected from the group comprising: lung adenocarcinoma (LUAD), colon adenocarcinoma (COAD), rectal adenocarcinoma (READ), and uterine corpus endometrial carcinoma (UCEC).

13. The method according to claim 1, wherein the inhibitor comprises a peptide IL-1 receptor antagonist, a nucleotide IL-1 receptor antagonist, peptide fragments of IL-1R1, an anti-IL-1 antibody, or a decoy IL-1 receptor.

14. The method according to claim 1, wherein the inhibitor comprises an amino acid sequence at least 90% identical to any one of SEQ ID NO: 1 (P01), SEQ ID NO: 2 (P02), SEQ ID NO: 3 (P03), SEQ ID NO: 4 (P04), or SEQ ID NO: 5 (P05).

15. The method according to claim 1, wherein the inhibitor is Isunakinra.

16. The method according to claim 1, wherein the subject is also administered an inhibitor of VCAN.

17. The method according to claim 16, wherein the inhibitor of VCAN is a toll-like receptor 1/2 (TLR1/2) inhibitor.

18. The method according to claim 1, wherein the subject is also administered one or more chemotherapeutic agent.

19. A pharmaceutical composition comprising:
an inhibitor of interleukin-1 receptor type 1 (IL-1R1) signalling; and
a pharmaceutically acceptable carrier.

20. (canceled)

21. A method of selecting a subject that has a cancer, which cancer is predicted to respond therapeutically to a treatment comprising an inhibitor of interleukin-1 receptor type 1 (IL-1R1) signalling, the method comprising:
determining whether the cancer comprises a KRAS mutation;
determining whether the cancer has an elevated level and/or activity of veriscan (VCAN); and
selecting the subject having a KRAS mutation and an elevated level and/or activity of VCAN for treatment comprising an inhibitor of interleukin-1 receptor type 1 (IL-1R1) signalling.

22. The method according to claim 21, wherein determining whether the cancer has an elevated level and/or activity of VCAN comprises determining whether the level and/or activity of VCAN in the cancer is elevated relative to a level and/or activity of VCAN in a reference sample.

23. The method according to claim 21, further comprising:
determining whether the cancer has an elevated level and/or activity of IL-1β, wherein the subject having a KRAS mutation, an elevated level and/or activity of VCAN, and has an elevated level and/or activity of IL-1β is selected for treatment comprising an inhibitor of interleukin-1 receptor type 1 (IL-1R1) signalling.

24. The method according to claim 23, wherein determining whether the cancer has an elevated level and/or activity of IL-1β comprises determining whether the level and/or activity of IL-1β in the cancer is elevated relative to a level and/or activity of IL-1β in a reference sample.

25. The method of claim 21, wherein at least one step is carried out in vitro and/or on a sample provided from the subject.

26. The method according to claim 21, the method further comprising treating the with an inhibitor of interleukin-1 receptor type 1 (IL-1R1) signalling.

27. A method comprising using veriscan (VCAN) as a biomarker for determining whether a subject having a cancer that comprises a KRAS mutation is suitable for treatment with an inhibitor of interleukin-1 receptor type 1 (IL-1R1) signalling.

28. A kit comprising:
a reagent for detecting the presence of a KRAS mutation; and
a reagent for determining the level and/or activity of VCAN.

29. The kit according to claim 28, further comprising:
a reagent for determining the level and/or activity of IL-1β.

30. (canceled)

31. The method of claim 13, wherein the decoy IL-1 receptor comprises a soluble IL-1 receptor, or an IL-1 TRAP.

32. The pharmaceutical composition of claim 19, wherein the inhibitor comprises a peptide IL-1 receptor antagonist, a nucleotide IL-1 receptor antagonist, peptide fragments of IL-1R1, an anti-IL-1 antibody, or a decoy IL-1 receptor.

33. The pharmaceutical composition of claim 32, wherein the decoy IL-1 receptor comprises a soluble IL-1 receptor, or an IL-1 TRAP.

34. The pharmaceutical composition of claim 19, wherein the inhibitor comprises an amino acid sequence at least 90% identical to any one of SEQ ID NO: 1 (P01), SEQ ID NO: 2 (P02), SEQ ID NO: 3 (P03), SEQ ID NO: 4 (P04), or SEQ ID NO: 5 (P05).

35. The pharmaceutical composition of claim 19, wherein the inhibitor is Isunakinra.

36. The method of claim 23, wherein at least one step is carried out in vitro and/or on a sample provided from the subject.


SEQUENCE LISTING (45 sequences)

SEQ ID NO: 1 - protein, 153 aa, Artificial Sequence
  P01
APVRSLAFRIWDVNQKTFYLRNNQLVAGYLQGPNVNLEEKIDVSFVQGEESNDKIPVALGIHGGKMCLSCVKSGDETRLQLEAVDPKNYPKKKMEKRFVFNKIEINNKLSFESAACPGWFLCTAMEADQPVSLTNMPDEGVMVTKFYMQFVSS

SEQ ID NO: 2 - protein, 153 aa, Artificial Sequence; identical to SEQ ID NO: 1
  P02

SEQ ID NO: 3 - protein, 153 aa, Artificial Sequence
  P03
APVRSLAFRIWDVNQKTFYLRNNQLVAGYLQGPNVNLEEKFSMSFVQGEESNDKIPVALGLKEKNLYLSCVLKDDKPTLQLESVDPKNYPKKKMEKRFVFIRSDSGPTTSFESAACPGWFLCTAMEADQPVSLTNMPDEGVMVTKFTMQFVSS

SEQ ID NO: 4 - protein, 153 aa, Artificial Sequence
  P04
APVRSLAFRIWDVNQKTFYLRNNQLVAGYLQGPNVNLEEKFSMSFVQGEESNDKIPVALGLKEKNLYLSCVLKDDKPTLQLESVDPKNYPKKKMEKRFVFNKIEINNKLEFESAACPGWFLCTAMEADQPVSLTNMPDEGVMVTKFTMQFVSS

SEQ ID NO: 5 - protein, 153 aa, Artificial Sequence
  P05
APVRSLNCRIWDVNQKTFYLRNNQLVAGYLQGPNVNLEEKFSMSFVQGEESNDKIPVALGLKEKNLYLSCVLKDDKPTLQLESVDPKNYPKKKMEKRFVFNKIEINNKLEFESAQFPNWFLCTAMEADQPVSLTNMPDEGVMVTKFYMQFVSS

SEQ ID NO: 6 - protein, 151 aa, Homo sapiens
  IL-1Ra (human)
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQED

SEQ ID NO: 7 - protein, 153 aa, Homo sapiens
  IL-1b (beta; human)
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 8 - DNA, 27 bp, Artificial Sequence
  Assay PCR primer - Kras exon 1F
tcttagagttttacacacaaaggtgag

SEQ ID NO: 9 - DNA, 18 bp, Artificial Sequence
  Assay PCR primer - Kras exon 1R
ttcaaagcggctggctgc

SEQ ID NO: 10 - DNA, 22 bp, Artificial Sequence
  Assay PCR primer - Kras exon 2F
ttgagctgtttacatcaccttg

SEQ ID NO: 11 - DNA, 27 bp, Artificial Sequence
  Assay PCR primer - Kras exon 2R
gcaaagaatcaataaatgtaagctatc

SEQ ID NO: 12 - DNA, 20 bp, Artificial Sequence
  Assay PCR primer - Kras exon 3F
gttgagggcaaggtttgaag

SEQ ID NO: 13 - DNA, 20 bp, Artificial Sequence
  Assay PCR primer - Kras exon 3R
acgaacaccatctggagcac

SEQ ID NO: 14 - DNA, 25 bp, Artificial Sequence
  Assay PCR primer - Mycoplasma Spp. F
gggagcaaacaggattagataccct

SEQ ID NO: 15 - DNA, 27 bp, Artificial Sequence
  Assay PCR primer - Mycoplasma Spp. R
tgcaccatctgtcactctgttaacctc

SEQ ID NO: 16 - DNA, 24 bp, Artificial Sequence
  Assay qPCR primer - mVcan F
ccagaacggaaatatcaagattgg

SEQ ID NO: 17 - DNA, 20 bp, Artificial Sequence
  Assay qPCR primer - mVcan R
tgaaacacaacaccatccac

SEQ ID NO: 18 - DNA, 22 bp, Artificial Sequence
  Assay qPCR primer - mIl1b F
tttgacagtgatgagaatgacc

SEQ ID NO: 19 - DNA, 20 bp, Artificial Sequence
  Assay qPCR primer - mIl1b R
aatgagtgatactgcctgcc

SEQ ID NO: 20 - DNA, 21 bp, Artificial Sequence
  Assay qPCR primer - mTnf F
gtccccaaagggatgagaagt

SEQ ID NO: 21 - DNA, 20 bp, Artificial Sequence
  Assay qPCR primer - mTnf R
tttgctacgacgtgggctac

SEQ ID NO: 22 - DNA, 22 bp, Artificial Sequence
  Assay qPCR primer - mGusb F
ttactttaagacgctgatcacc

SEQ ID NO: 23 - DNA, 20 bp, Artificial Sequence
  Assay qPCR primer - mGusb R
acctccaaatgcccatagtc

SEQ ID NO: 24 - DNA, 20 bp, Artificial Sequence
  Assay qPCR primer - hACTB F
gagcacagagcctcgccttt

SEQ ID NO: 25 - DNA, 21 bp, Artificial Sequence
  Assay qPCR primer - hACTB R
tcatcatccatggtgagctgg

SEQ ID NO: 26 - DNA, 22 bp, Artificial Sequence
  Assay qPCR primer - hVCAN F
gaatgtcactctaatccctgtc

SEQ ID NO: 27 - DNA, 20 bp, Artificial Sequence
  Assay qPCR primer - hVCAN R
tgtctcggtatcttgctcac

SEQ ID NO: 28 - DNA, 19 bp, Artificial Sequence
  Vcan (shVcan, sc-41904 V) shRNA target sequence 1
caaccaccatcgaaatgaa

SEQ ID NO: 29 - DNA, 19 bp, Artificial Sequence
  Vcan (shVcan, sc-41904 V) shRNA target sequence 2
ctcacaggctgtaattaca

SEQ ID NO: 30 - DNA, 19 bp, Artificial Sequence
  Vcan (shVcan, sc-41904 V) shRNA target sequence 3
gtagacatatcccatacta

SEQ ID NO: 31 - DNA, 19 bp, Artificial Sequence
  Kras (shKras, sc-43876-V) shRNA target sequence 1
ctacaggaaacaagtagta

SEQ ID NO: 32 - DNA, 19 bp, Artificial Sequence
  Kras (shKras, sc-43876-V) shRNA target sequence 2
gaacagtagacacgaaaca

SEQ ID NO: 33 - DNA, 19 bp, Artificial Sequence
  Kras (shKras, sc-43876-V) shRNA target sequence 3
ccattcagtttccatgtta

SEQ ID NO: 34 - DNA, 21 bp, Artificial Sequence
  Chuk (shChuk, sc-29366-V) shRNA target sequence 1
ccatggtgtttgaatgtattt

SEQ ID NO: 35 - DNA, 21 bp, Artificial Sequence
  Chuk (shChuk, sc-29366-V) shRNA target sequence 2
ctctcagtgtgttctagattt

SEQ ID NO: 36 - DNA, 21 bp, Artificial Sequence
  Chuk (shChuk, sc-29366-V) shRNA target sequence 3
gcaagcagaagattattgatt

SEQ ID NO: 37 - DNA, 21 bp, Artificial Sequence
  Ikbkb (shIkbkb, sc-35645-V) shRNA target sequence 1
gatgacatcttgaacttgatt

SEQ ID NO: 38 - DNA, 21 bp, Artificial Sequence
  Ikbkb (shIkbkb, sc-35645-V) shRNA target sequence 2
ctgcacatttgaatctgtatt

SEQ ID NO: 39 - DNA, 21 bp, Artificial Sequence
  Ikbkb (shIkbkb, sc-35645-V) shRNA target sequence 3
cagctctcttagacagttatt

SEQ ID NO: 40 - DNA, 21 bp, Artificial Sequence
  Ikbke (shIkbke, sc-39057-V) shRNA target sequence 1
gagatcatgtacagaatcatt

SEQ ID NO: 41 - DNA, 21 bp, Artificial Sequence
  Ikbke (shIkbke, sc-39057-V) shRNA target sequence 2
cagtgttgtttggacaagatt

SEQ ID NO: 42 - DNA, 21 bp, Artificial Sequence
  Ikbke (shIkbke, sc-39057-V) shRNA target sequence 3
ccaacaaactagcattacttt

SEQ ID NO: 43 - DNA, 21 bp, Artificial Sequence
  Tbk1 (shTbk1, sc-39059-V) shRNA target sequence 1
gtaggactgagatatgaaatt

SEQ ID NO: 44 - DNA, 21 bp, Artificial Sequence
  Tbk1 (shTbk1, sc-39059-V) shRNA target sequence 2
gcatcacagagatttactatt

SEQ ID NO: 45 - DNA, 21 bp, Artificial Sequence
  Tbk1 (shTbk1, sc-39059-V) shRNA target sequence 3
gaagttctagtttgcacaatt